(19) United States
(12) Patent Application Publication
(10) Pub. No.: US 2017/0258967 A1
(43) Pub. Date: Sep. 14, 2017

(54) COMPOSITIONS AND METHODS FOR DELIVERING AN AGENT TO A WOUND

(71) Applicant: UNIVERSITY OF MEMPHIS RESEARCH FOUNDATION, MEMPHIS, TN (US)

(72) Inventors: Warren O. HAGGARD, MEMPHIS, TN (US); Ashley C. PARKER, MEMPHIS, TN (US); Jessica Amber JENNINGS, MEMPHIS, TN (US)

(73) Assignee: UNIVERSITY OF MEMPHIS RESEARCH FOUNDATION, MEMPHIS, TN (US)

(21) Appl. No.: 15/118,414

(22) PCT Filed: Feb. 13, 2015
(86) PCT No.: PCT/US15/15776

§ 371 Date: Aug. 11, 2016

Related U.S. Application Data
(60) Provisional application No. 61/940,296, filed on Feb. 14, 2014.
(60) Provisional application No. 62/077,047, filed on Nov. 7, 2014.

(51) Int. Cl. A61L 29/16; A61K 47/10; A61L 29/08; A61L 29/04; A61L 29/14; C08J 5/18; A61K 9/00; A61K 38/14; A61K 31/7036; C08L 5/08; C08L 71/02; A61K 47/36; A61K 31/7048

(52) U.S. Cl. CPC A61L 29/16; A61K 47/36; A61K 47/10; A61L 29/085; A61L 29/049; A61L 29/146; A61K 31/7048; A61K 9/0024; A61L 29/148; A61K 38/14; A61K 31/7036; C08L 5/08; C08L 71/02; C08J 5/18; A61L 2300/404; A61L 2300/232; A61L 2300/252; A61L 2420/06; A61L 2300/606; C08L 2203/02; C08J 2305/08; C08J 2371/02

(57) ABSTRACT

The invention provides compositions featuring chitosan and polyethylene glycol and methods for using such compositions for the local delivery of biologically active agents to an open fracture, complex wound or other site of infection. Advantageously, the chitosan-PEG compositions can be loaded with one or more antimicrobial agents, including hydrophobic agents, and can be tailored to the needs of particular patients at the point of care (e.g., in a surgical suite, clinic, physician's office, or other clinical setting).

### CROSS REFERENCE TO RELATED APPLICATIONS

This application claims benefit of U.S. Provisional Application Ser. No. 61/940,296, filed Feb. 14, 2014, and 62/077,047, filed Nov. 7, 2014, the contents of each of which are incorporated herein by reference in their entirety.

### STATEMENT OF RIGHTS TO INVENTIONS MADE UNDER FEDERALLY SPONSORED RESEARCH

This invention was made with U.S. government support under grant no. DM090455 awarded by United States Army Medical Research Acquisition Activity (USAMRRAA); Grant Nos.: W81CWH-09-PROP-HAD and W81XWH-12-2-0020 awarded by United States Army Medical Research and Materiel Command (USAMRMC); Grant Nos. W81XWH-08-1-0312, Proposal No. 07128022, and W81XWH-12-2-0020, Proposal No. DM090455 awarded by the Department of Defense; and Grant No. 2009087439 awarded by the National Science Foundation. The U.S. government has certain rights in the invention.

### BACKGROUND OF THE INVENTION

Musculoskeletal injuries are some of the most prevalent injuries in both civilian (e.g., open fractures) and military (e.g., extremity injuries) populations and their infections can be difficult to treat, often resulting in multiple surgeries and increased costs. Fungal infections have recently been increasingly problematic in complex extremity trauma and have significantly higher amputation rates than those from bacterial infections. Many fungal strains adhere to tissue and implanted orthopedic hardware within wounds to form biofilms, and can easily spread to become prominent resistant infections. Invasive Candida albicans (C. albicans) infection is the third most common cause of hospital-acquired bloodstream infections. These problematic, often polymicrobial, infections can result in high healthcare costs, high mortality rates, and significantly higher amputation rates than those from bacterial infections alone. In 2010, 78% of the wounded U.S. soldiers in Afghanistan with IFI required lower extremity amputations. An outbreak of cutaneous mucormycosis was also recently reported in victims from the 2011 tornado in Joplin, Mo., where 38% of the infected patients died.

Wound infections may also be complicated by the presence of multiple bacteria and fungi and by the ability of microbes to form biofilms, which raise the minimum inhibitory concentration (MIC) of antimicrobial agents. Local antimicrobial delivery releases high levels of antimicrobials directly to injured wound tissue, overcoming sub-bactericidal or sub-fungicidal antimicrobial levels present in the avascular wound zones. While many local antibiotic delivery systems exist on the market, there are no commercially available local antifungal delivery systems. Rather, invasive fungal infections are treated with systemic antifungal delivery.

Systemic toxicity can be a concern with many of the antifungals used to treat fungal infections, so local wound delivery is highly desirable. However, development of local antifungal delivery systems has been minimal, typically due to the hydrophobicity of antifungals. These hydrophobic antifungals can be difficult to incorporate into and release from more hydrophilic local drug delivery systems.

Because current methods for treating or preventing infection, particularly fungal infections related to musculoskeletal injuries, are inadequate, compositions and methods for providing agents to prevent or treat an infection at a site of trauma are urgently required.

### SUMMARY OF THE INVENTION

As described below, the present invention features compositions comprising chitosan and polyethylene glycol that provide for the delivery of hydrophobic agents, alone or in combination with hydrophilic agents.

In one aspect, the invention provides a method for producing a biodegradable composition containing chitosan and polyethylene glycol, the method involving dissolving polyethylene glycol having a molecular weight of at least about 2-12,000 g/mol and chitosan; and forming the mixture of chitosan and polyethylene glycol into a desired shape under conditions that reduce the water content by about 10%-100% (10, 20, 30, 40, 50, 60, 70, 80, 90, 100%). In one embodiment, the invention further includes incorporating an effective amount of one or more agents into the composition.

In another aspect, the invention provides a method for producing a biodegradable composition containing chitosan, polyethylene glycol, and one or more agents selected by a clinician at a point of care, the method involving dissolving polyethylene glycol having a molecular weight of at least about 2-12,000 g/mol and chitosan in one or more acids in a solvent to form a mixture, forming the mixture of chitosan and polyethylene glycol into a desired shape under conditions that reduce the water content by about 10%-100%; selecting one or more agents; and incorporating an effective amount of the agent into the composition at a point of care.

In another aspect, the invention provides a biodegradable composition containing chitosan and polyethylene glycol produced by the method of any previous aspect.

In another aspect, the invention provides a wound management device containing chitosan and polyethylene glycol produced by the method of any previous aspect.

In another aspect, the invention provides a chitosan-PEG composition containing or consisting essentially of chitosan having a degree of deacetylation of at least about 51% and polyethylene glycol having a molecular weight of at least about 6,000 g/mol, where the water content is about 0-90% (0, 10, 20, 30, 40, 50, 60, 70, 90%).

In another aspect, the invention provides a wound management device containing a chitosan-PEG composition, the composition containing or consisting essentially of chitosan having a degree of deacetylation of at least about 51% and polyethylene glycol having a molecular weight of at least about 6,000 g/mol, where the water content is about 0-90%, and an effective amount of an agent.

In another aspect, the invention provides a method for treating or preventing an infection in a subject at a site of trauma, the method involving contacting the site with a wound management device containing or consisting essentially of a chitosan-PEG composition of any previous aspect and an effective amount of at least one agent selected and incorporated at a point of care.

In another aspect, the invention provides a method for the local delivery of an agent to a site, the method involving contacting the site with a chitosan-PEG composition containing an agent, thereby delivering the agent to the site. In particular embodiments, the chitosan-PEG composition releases about 2 μg-1000 mg of the agent in 1-72 hours (e.g., about 100-200 μg in about 1 hour, about 200-400 μg in about 3 hours, about 250-500 μg in about 6 hours, about 350-700 μg in about 24 hours, about 500-800 μg in about 48 hours, about 600-1000 μg in about 72 hours.

In another aspect, the invention provides a medical device for implantation containing an acid-treated chitosan-PEG composition, the composition containing or consisting essentially of chitosan having a degree of deacetylation of at least about 51% and polyethylene glycol having a molecular weight of at least about 6,000 g/mol, and further involving an effective amount of an agent. In one embodiment, the chitosan-PEG composition is a film that adheres to the device. In another embodiment, the device contains plastic, silicone, titanium, or stainless steel. In another embodiment, the medical device is a catheter or heart valve.

In another aspect, the invention provides a kit containing a chitosan-PEG composition of any previous aspect for use in treating a trauma site or delivering an agent. In one embodiment, the chitosan-PEG composition is present in a wound management device or medical device for implantation. In another embodiment, the chitosan-PEG composition is in the form of a plug, mesh, strip, suture, dressing, sponge, film, hydrogel, or combinations thereof. In another embodiment, the chitosan-PEG composition contains an agent selected from the group consisting of antimicrobial agent, growth factor, anti-inflammatory, clot promoting agent, anti-thrombotic, and anticancer agent. In another embodiment, the agent is a hydrophobic and/or hydrophilic compound.

In various embodiments of any previous aspect or any other aspect of the invention delineated herein, the chitosan-PEG composition is in the form of a film, hydrogel, mesh, plug, strip, sponge, suture, dressing, or combinations thereof. In various embodiments of any previous aspect or any other aspect of the invention delineated herein, the agent is a hydrophobic and/or hydrophilic compound. In various embodiments of any previous aspect or any other aspect of the invention delineated herein, the agent is selected from the group consisting of antimicrobial, growth factor, anti-inflammatory, hemostatic, and anti-thrombotic agents. In various embodiments of any previous aspect or any other aspect of the invention delineated herein, the antimicrobial agent is selected from the group consisting of antifungal, antibacterial, and antiviral agents. In various embodiments of any previous aspect or any other aspect of the invention delineated herein, the antimicrobial agents are amphotericin B, vancomycin, and/or amikacin. In still other embodiments of any previous aspect or any other aspect of the invention delineated herein, the effective amount of the agent is sufficient to reduce the survival or proliferation of a bacterial or fungal cell. In other embodiments of any previous aspect or any other aspect of the invention delineated herein, the fungal cell is Candida albicans and/or the bacterial cell is Pseudomonas aeruginosa (lux) or Staphylococcus aureus. In various embodiments of any previous aspect or any other aspect of the invention delineated herein, the composition releases at least about 0.2-50 ng of an antimicrobial agent per hour. In various embodiments of any previous aspect or any other aspect of the invention delineated herein, the chitosan degree of deacetylation is at least about 61, 71, or 81 percent. In various embodiments of any previous aspect or any other aspect of the invention delineated herein, the polyethylene glycol has a molecular weight of at least about 6,000, 8,000, or 10,000 g/mol. In various embodiments of any previous aspect or any other aspect of the invention delineated herein, the composition is biodegradable over at least about one, two, three, four, five, seven, ten, fourteen, twenty-one, or twenty-eight days. In various embodiments of any previous aspect or any other aspect of the invention delineated herein, the degree of deacetylation of chitosan and/or the molecular weight of polyethylene glycol is varied to customize the composition's degradation and elution rates. In various embodiments of any previous aspect or any other aspect of the invention delineated herein, the composition is custom loaded with an agent by a clinician at the point of treatment. In particular embodiments, the hydrophobic or hydrophilic compound is an antibacterial, antifungal, or anticancer agent. In various embodiments of any previous aspect or any other aspect of the invention delineated herein, the hydrophobic compound is selected from the group consisting of amphotericin B, voriconazole, rifampcin, erthromycin, novobiocin, fusidic acid, paclitaxel, and a steroid; and the hydrophilic compound is selected from the group consisting of is selected from the group consisting of vancomycin, amikacin, daptomycin, gentimicin, tobramycin, Anasept, penicillin and derivatives. In various embodiments of any previous aspect or any other aspect of the invention delineated herein, the water content is reduced by one, two, or more lyophilization steps. In various embodiments of any previous aspect or any other aspect of the invention delineated herein, the method further involves neutralizing the lyophilized sponge in NaOH, washing the sponge to neutralize it, and freezing the sponge and lyophilizing a second time. In various embodiments of any previous aspect or any other aspect of the invention delineated herein, the one or more agents is selected based on the source of trauma and/or infection. In particular embodiments of the above aspects, the method is ex vivo. In particular embodiments of the above aspects, the composition is a wound management device. In particular embodiments of the above aspects, the desired shape is obtained by freezing the mixture of chitosan and polyethylene glycol in a mold and lyophilizing to form a sponge. In particular embodiments of the above aspects, the desired shape is obtained by pouring the mixture of chitosan and polyethylene glycol into a thin layer and heating the chitosan to form a dehydrated film. In particular embodiments of the above aspects, the chitosan-PEG composition is molded to form a plug, mesh, strip, suture, dressing, sponge, or film. In particular embodiments of the above aspects, the one or more acids are selected from the group consisting of acetic, citric, oxalic, proprionic, ascorbic, hydrochloric, formic, salicylic and lactic acids. In particular embodiments of the above aspects, the chitosan-PEG composition biodegrades over at least about 2-28 days when implanted in a subject. In particular embodiments of the above aspects, the degree of deacetylation of chitosan and/or the molecular weight of polyethylene glycol is varied to customize the composition's degradation and elution rates. In particular embodiments of the above aspects, the composition containing an effective amount of an agent selected at the point of care. In particular embodiments of the above aspects, the composition releases at least about 0.2-50 ng of an antimicrobial agent per hour. In particular embodiments of the above aspects, the method reduces fungi or bacteria present at the site by at least about 20-100% at 72 hours after contact with the chitosan-PEG composition relative to an untreated control site. In particular embodiments of the above aspects, the point of treatment is in a surgical suite, clinic, physician's office, or other clinical setting.

The invention provides compositions featuring chitosan and polyethylene glycol and methods for using such compositions for the local delivery of biologically active agents (e.g., antifungal agents) to an open fracture, complex wound or other site of infection. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “chitosan” is meant a chitin-derived polymer that is at least 20% deacetylated. In various embodiments, chitosan is at least about 50% deacetylated. In particular embodiments, chitosan is at least about 61% or 71% deacetylated. Chitin is a linear polysaccharide consisting of (1-4)-linked 2-acetamido-2-deoxy-b-D-glucopyranose. Chitosan is a linear polysaccharide consisting of (1-4)-linked 2-amino-2-deoxy-b-D-glucopyranose. An exemplary chitosan polymer is shown by the formula below. In one embodiment, chitosan has a molecular weight of about 250 kD.

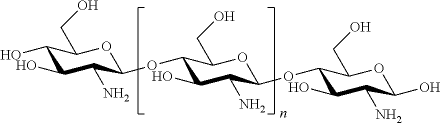

By “acid treated chitosan” is meant chitosan that is solubilized in an acidic solution.

By “amphotericin B” is meant the compound (1R,3S,5R,6R,9R,11R,15S,16R,17R,18S,19E,21E,23E,25E,27E,29E,31E,33R,35S,36R,37S)-33-[(3-amino-3,6-dideoxy-β-D-mannopyranosyl)oxy]-1,3,5,6,9,11,17,37-octahydroxy-15,16,18-trimethyl-13-oxo-14,39-dioxabicyclo [33.3.1] nonatriaconta-19,21,23,25,27,29,31-heptaene-36-carboxylic acid and CAS number 1397-89-3. Amphotericin B is shown by the formula below.

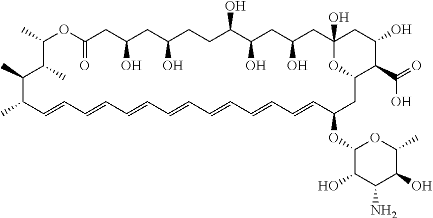 [SMILES: C[C@@H]1[C@H](C)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)O]

By “polyethylene glycol (PEG)” is meant an oligomer or polymer of ethylene oxide. Commercially available PEG ranges in molecular weight from 300 g/mol to 10,000,000 g/mol. An exemplary PEG is shown by the formula below.

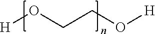

In particular embodiments, PEG molecular weight is 6000 g/mol, 8,000 g/mol, 10,000 g/mol. The degradation profile of the chitosan/PEG composition can be tailored to the desired level by increasing or decreasing the molecular weight of the PEG. In particular, when lower molecular weight PEG is used degradation is enhanced. When higher molecular weight PEG is used degradation is decreased.

By “degrades” is meant physically or chemically breaks down in whole or in part. Preferably, the degradation represents a physical reduction in the mass by at least about 10%, 25%, 50%, 75%, 80%, 85%, 90%, 95% or 100%.

By “long term release” is meant elution of an agent over the course of twenty-four-seventy-two hours or longer. In particular embodiments, release occurs over one, two, three or four weeks.

By “sponge” is meant a three-dimensional porous matrix. In particular embodiments, the sponge contains independent pores. In particular embodiments, the sponge does not contain vertically oriented pores. In other embodiments, the sponge is obtained by uniformly freezing the chitosan/PEG composition.

By “wound management device” or “wound healing device” is meant any material used to protect or promote healing at a site of trauma.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a chitosan analog retains the biological activity of a corresponding reference chitosan polymer (e.g., manufactured chitosan), while having certain biochemical modifications that enhance the analog's function relative to a reference chitosan polymer. Such biochemical modifications could increase the analog's ability to be degraded, to uptake or elute a therapeutic agent, or to increase or decrease mechanical strength.

By “antimicrobial” is meant an agent that inhibits or stabilizes the proliferation or survival of a microbe. In one embodiment, a bacteriostatic agent is an antimicrobial. In other embodiments, any agent that kills a microbe (e.g., bacterium, fungus, virus) is an antimicrobial.

By “biodegradable” is meant susceptible to breakdown by biological activity. For example, biodegradable chitosan-PEG compositions are susceptible to breakdown by enzymes present in vivo (e.g., lysozyme, N-acetyl-o-glucosaminidase and lipases). Degradation of a chitosan-PEG composition of the invention need not be complete. A chitosan-PEG composition of the invention may be degraded, for example, by the cleavage of one or more chemical bonds (e.g., glycosidic bonds).

By “clinician” is meant any healthcare provider. Exemplary clinicians include, but are not limited to, doctors, veterinarians, osteopaths, physician's assistants, emergency medical technicians, medics, nurse practitioners, and nurses.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “decreases” is meant a negative alteration of at least 10%, 25%, 50%, 75%, 100%, 200%, 300%, 400%, 500%, 1000%, or more.

“Detect” refers to identifying the presence, absence or amount of the analyte to be detected.

By “customize” is meant tailor to suit the needs of a particular subject.

By “degradation rate” is meant the time required to substantially degrade the composition. A composition is substantially degraded where at least about 75%, 85%, 90%, 95% or more has been degraded. Methods for measuring degradation of chitosan are known in the art and include measuring the amount of a sponge, film, composite or other composition of the invention that remains following implantation in a subject or following in vitro exposure to an enzyme having chitosan-degrading activity.

By “disease” is meant any condition or disorder that damages or interferes with the normal function of a cell, tissue, or organ. In one embodiment, the disease is a bacterial infection, fungal infection, or a combination thereof (e.g., a biofilm) present at a wound site.

By “effective amount” is meant the amount of an agent required to ameliorate the symptoms of a disease relative to an untreated patient. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “elution rate” is meant the time required for an agent to be substantially released from a composition. Elution can be measured by determining how much of an agent remains within the composition or by measuring how much of an agent has been released into the composition's surroundings. Elution may be partial (10%, 25%, 50%, 75%, 80%, 85%, 90%, 95% or more) or complete. In one preferred embodiment, the agent continues to be released at an effective level for at least about 3, 4, 5, 6, 7, 8, 9, or 10 days.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, “hydrophilic” is meant to refer to any compound, molecule, or biologically active agent that is relatively soluble in water and/or has high affinity for water.

As used herein, “hydrophobic” is meant to refer to any compound, molecule, or biologically active agent that is relatively insoluble in water and/or has low affinity for water.

By “infection” is meant the presence of one or more pathogens in a tissue or organ of a host. An infection includes the proliferation of a microbe (e.g., bacteria, viruses, fungi) within a tissue of a subject at a site of trauma.

By “increases” is meant a positive alteration of at least 10%, 25%, 50%, 75%, 100%, 200%, 300%, 400%, 500%, 1000%, or more.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “point of treatment” is meant the site where healthcare is delivered. A “point of treatment” includes, but is not limited to, a surgical suite, physician's office, clinic, or hospital.

By “polymer” is meant a natural or synthetic organic molecule formed by combining smaller molecules in a regular pattern.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “profile” is meant a set of characteristics that define a composition or process. For example, a “biodegradation profile” refers to the biodegradation characteristics of a composition. In another example, an “elution profile” refers to elution characteristics of a composition.

By “prosthetic device” is meant an implanted medical device that substitutes for or supplements a missing or defective part of the body.

By “reference” is meant a standard or control condition.

By “small molecule” is meant any chemical compound.

By “subject” is meant a mammal, including, but not limited to, a human or non-human mammal, such as a bovine, equine, canine, ovine, or feline.

By “trauma” is meant any injury that damages a tissue or organ of a subject. The injury need not be severe. Therefore, a trauma includes any injury that breaks the skin.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural. Thus, for example, reference to “an amino acid substitution” includes reference to more than one amino acid substitution.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other features and advantages of the invention will be apparent from the following description of the desirable embodiments thereof, and from the claims.

### BRIEF DESCRIPTION OF THE DRAWINGS

FIG. 1 is a graph depicting the in vitro degradation of chitosan-PEG sponge formulations.

FIG. 2 is a graph depicting Amphotericin B (water insoluble) elution from chitosan-PEG sponge formulations.

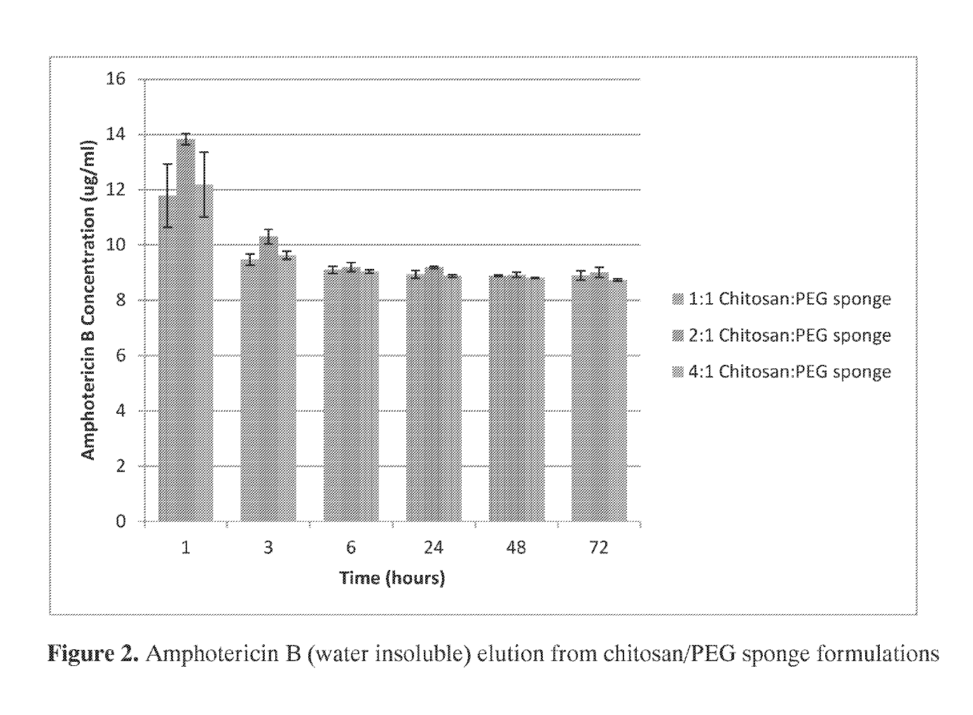

FIG. 3 is a graph depicting in vitro degradation of chitosan-PEG sponge formulations after 4 days in 1 mg/ml lysozyme solution. All sponges were made with 1% blended acid unless otherwise noted.

FIG. 4 is a graph depicting Amphotericin B (sodium deoxycholate solubilized) elution from second round of chitosan-PEG sponge formulations.

FIG. 5 is a graph depicting swelling of chitosan-PEG sponge formulations in water.

FIG. 6 is a graph depicting swelling of chitosan-PEG sponge formulations in water (LA: lactic/acetic acid).

FIG. 7 are images of chitosan-PEG sponge formulations.

FIG. 8 is a graph depicting Amphotericin B (sodium deoxycholate solubilized) released from chitosan-PEG sponge formulations. 0.25 micrograms Amphotericin B was used per ml Candida.

FIG. 9 is a graph depicting release of Amphotericin B (sodium deoxycholate solubilized) from chitosan-PEG sponge formulations.

FIG. 10 are images of chitosan-PEG sponge formulations after amphotericin B elution.

FIG. 11A depicts stacked ATR-FTIR (attenuated total reflection Fourier transform infrared spectroscopy) absorbance spectra for I. chitosan/PEG 6000 1 lyo, II. chitosan/PEG 8000 1 lyo, III. chitosan/PEG 8000 2 lyo, IV. chitosan/PEG 6000 2 lyo, and V. 1% chitosan sponges (average; n=3). FIG. 11B depicts stacked ATR-FTIR absorbance spectra for I. chitosan powder, II. PEG 6000, and III. PEG 8000 (average; n=3).

FIG. 12A depicts stacked x-ray diffraction (XRD) spectra in the 20 range from 5° to 40° for (top to bottom) the chitosan powder, chitosan/PEG 6000 1 lyo sponge, chitosan/PEG 8000 2 lyo sponge, chitosan/PEG 6000 2 lyo sponge, 1% chitosan sponge, and chitosan/PEG 8000 1 lyo sponge. FIG. 12B depicts XRD spectra in the 20 range from 5° to 40° for PEG 6000. FIG. 12C depicts XRD spectra in the 20 range from 5° to 40° for PEG 8000.

FIG. 13A depicts representative scanning electron microscopy of 1% chitosan sponge cross sections at 30× magnification. FIG. 13B depicts representative scanning electron microscopy of chitosan/PEG 6000 1 lyo sponge cross sections at 30× magnification. FIG. 13C depicts representative scanning electron microscopy of chitosan/PEG 6000 2 lyo sponge cross sections at 30× magnification. FIG. 13D depicts representative scanning electron microscopy of chitosan/PEG 8000 1 lyo sponge cross sections at 30× magnification.

FIG. 13E depicts representative scanning electron microscopy of chitosan/PEG 8000 2 lyo sponge cross sections at 30× magnification. FIG. 13F depicts representative scanning electron microscopy of 1% chitosan sponge surface structure at 30× magnification. FIG. 13G depicts representative scanning electron microscopy of chitosan/PEG 6000 1 lyo sponge surface structure at 30× magnification. FIG. 13H depicts representative scanning electron microscopy of chitosan/PEG 6000 2 lyo, surface structure at 30× magnification. FIG. 13I depicts representative scanning electron microscopy of chitosan/PEG 8000 1 lyo sponge surface structure at 30× magnification. FIG. 13J depicts representative scanning electron microscopy of chitosan/PEG 8000 2 lyo sponges sponge surface structure at 30× magnification.

FIG. 14 shows six scanning electron microscopy images of cross sections of sponges.

FIG. 15 shows six scanning electron microscopy images of sponge faces.

FIG. 16 shows scanning electron microscopy images of sponge layers.

FIG. 17 is a graph depicting in vitro degradation of chitosan-PEG sponge formulations after 4 days in 1 mg/ml lysozyme solution.

FIG. 18A is a graph depicting percent sponge remaining (mean±standard deviation) of chitosan/PEG 6000 1 lyo (o), chitosan/PEG 6000 2 lyo (▪), chitosan/PEG 8000 1 lyo (▴), chitosan/PEG 8000 2 lyo (♦), and 1% chitosan () sponges after 2, 4, 8, or 10 days of in vitro enzymatic-mediated degradation (n=3), where at each time point, * indicates p<0.05 versus 1% chitosan, † denotes p<0.05 versus chitosan/PEG 8000 2 lyo, ** indicates p<0.05 versus all, and ‡ represents p<0.05 versus all except §. FIG. 18B is a graph depicting Brookfield viscosity measurements of sponges dissolved in 0.1 M sodium acetate and 0.2 M acetic acid before degradation (day 0) and after enzymatic-mediated degradation (day 10), where * denotes p<0.05 pairwise.

FIG. 19 is a graph depicting amphotericin B release in vitro in μg/mL (mean±standard deviation) from sponges over time and the minimum inhibitory concentration of amphotericin B for Candida albicans, where * represents p<0.001 versus chitosan/PEG 6000 1 lyo sponge 1 hour time point, † denotes p<0.003 versus chitosan/PEG 8000 1 lyo sponge 1 hour time point, δ indicates p<0.05 versus 1% chitosan sponge 1 hour time point, ** represents p<0.001 versus all sponge groups within the time point, and †† denotes p<0.007 versus all sponge groups within the time point except for ‡.

FIG. 20 is a graph depicting Amphotericin B release from Chitosan-PEG sponges and 1% chitosan sponges over 72 hours inhibited Candida albicans in a zone of inhibition assay. The concentration of Amphotericin B was determined using Amphotericin B standards on plates of Candida albicans; the distance of fungal inhibition was measured and correlated to the Amphotericin B concentration. * indicates p<0.05 versus control 1% chitosan sponge.

FIG. 21 is a graph depicting direct contact biocompatibility normalized to polyurethane sponge control, represented in mean percent cell viability±standard deviation, of normal human dermal fibroblasts after exposure to polyurethane, chitosan/PEG, and 1% chitosan sponges for one and three days, analyzed using Cell Titer-Glo® luminescent assay. (* indicates p<0.05 versus polyurethane sponge control).

FIG. 22 shows six images of normal human dermal fibroblasts exposed for one day to a control polyurethane sponge, 1% chitosan sponge, and four chitosan polyethylene glycol sponge variations, where green and red represent living and dead cells, respectively.

FIG. 23 is a histological analysis boxplot of the percentage of sponge implants present in the defect area, 4 and 10 days after surgery. (+ and o represents the mean and a data point outside of the standard deviation, respectively).

FIG. 24 is a histological analysis boxplot of the average graded inflammatory response from three blinded reviewers, cellular response scores were 0—no tissue for evaluation, 1—very mild leukocytic density, 2—slightly more elevated cellular response, 3—moderate cellular response, 4—high cell density in and around implant site, and 5—extremely high cellular response. (+ and o represents the mean and a data point outside of the standard deviation, respectively).

FIG. 25 depicts loading of chitosan-PEG sponges with antimicrobial agents to bacterial and fungal microorganisms infecting a wound at point of care.

FIG. 26 is a graph depicting sodium deoxycholate solubilized amphotericin B release in vitro in μg/ml (mean±standard deviation) from single loaded and dual-loaded (with vancomycin) sponges over time (n=3), where * indicates p<0.05 pairwise overall, and † represents p<0.05 pairwise at the respective time point.

FIG. 27A is a graph depicting vancomycin release in vitro in μg/ml (mean±standard deviation) from single loaded and dual-loaded (with amphotericin B) sponges over time (n=3), where at each respective time point, * indicates p<0.001 versus other loading condition within sponge group, † represents p<0.05 versus all other sponges, and ** denotes p<0.001 versus all sponge types in loading condition. FIG. 27B is an inset of the graph depicted in FIG. 27A, showing elution times 6 hours.

FIG. 28 is a graph depicting results of antifungal activity zone of inhibition assay using amphotericin B eluted from single-loaded and dual-loaded chitosan/PEG and chitosan sponges, where * indicates p=0.001 and † represents p=0.002 pairwise.

FIG. 29A is a graph depicting cytocompatibility (mean±standard deviation) of single loaded amphotericin B eluates (n=3) from the chitosan/PEG 6000 sponge to normal human dermal fibroblasts after one and three days. FIG. 29B is a graph depicting cytocompatibility (mean±standard deviation) of single loaded amphotericin B eluates (n=3) from the chitosan/PEG 8000 sponge to normal human dermal fibroblasts after one and three days. FIG. 29C is a graph depicting cytocompatibility (mean±standard deviation) of single loaded amphotericin B eluates (n=3) from t control chitosan sponge to normal human dermal fibroblasts after one and three days. Cells were analyzed using Cell Titer-Glo® luminescent assay and resulting cell numbers were converted to percent cell viability by normalization to tissue culture plastic (TCP) controls. (* indicates p<0.05 versus TCP controls) FIG. 30A is a graph depicting cytocompatibility (mean±standard deviation) of single loaded vancomycin eluates (n=3) from the chitosan/PEG 6000 sponge to normal human dermal fibroblasts after one and three days. FIG. 30B is a graph depicting cytocompatibility (mean±standard deviation) of single loaded vancomycin eluates (n=3) from the chitosan/PEG 8000 sponge to normal human dermal fibroblasts after one and three days. FIG. 30C is a graph depicting cytocompatibility (mean±standard deviation) of single loaded vancomycin eluates (n=3) from control chitosan sponge to normal human dermal fibroblasts after one and three days. Cells were analyzed using Cell Titer-Glo® luminescent assay and resulting cell numbers were converted to percent cell viability by normalization to tissue culture plastic (TCP) controls. (* indicates p<0.05 versus TCP controls)

FIG. 31A is a graph depicting cytocompatibility (mean±standard deviation) of dual-loaded vancomycin and amphotericin B eluates (n=3) from the chitosan/PEG 6000 sponge to normal human dermal fibroblasts after one and three days. FIG. 31B is a graph depicting cytocompatibility (mean±standard deviation) of dual-loaded vancomycin and amphotericin B eluates (n=3) from the chitosan/PEG 8000 sponge to normal human dermal fibroblasts after one and three days. FIG. 31C is a graph depicting cytocompatibility (mean±standard deviation) of dual-loaded vancomycin and amphotericin B eluates (n=3) from control chitosan sponge to normal human dermal fibroblasts after one and three days. Cells were analyzed using Cell Titer-Glo® luminescent assay and resulting cell numbers were converted to percent cell viability by normalization to tissue culture plastic (TCP) controls. (* indicates p<0.05 versus TCP controls)

FIG. 32 is a graph depicting percentage of catheters cleared of S. aureus for catheters retrieved from mice treated with chitosan/PEG 6000, chitosan/PEG 8000, and chitosan sponges (four animals and eight catheters).

FIGS. 33A and 33B are graphs depicting the average colony forming units (CFUs) per catheter for catheters retrieved from mice treated with chitosan/PEG 6000, chitosan/PEG 8000, and chitosan sponges (four animals and eight catheters). FIG. 33B depicts the results in FIG. 33A on a logarithmic scale. Sponges were loaded with either PBS or 4 mg/ml of vancomycin (Vanc) * indicates p<0.05 versus PBS loaded sponges.

### DETAILED DESCRIPTION OF THE INVENTION

As described below, the present invention features compositions comprising a combination of chitosan and polyethylene glycol (PEG) (e.g., a sponge) that provide for the local delivery of biologically active agents (e.g., hydrophobic and antifungal agents) and methods of using such compositions to treat or prevent an infection (e.g., fungal) or promote healing.

Previous research conducted has yielded a topical, porous, degradable chitosan sponge that provides an adaptable antibiotic delivery system in which tailored dosing and degradation can be achieved. This system can be loaded with antibiotics immediately prior to use, allowing for clinician selected antibiotic loading, as well as combination loading. However, hydrophobic agents are difficult to load in the chitosan sponges (i.e., that lack polyethylene glycol). The invention is based, at least in part, on the discovery that the addition of polyethylene glycol as a component of the chitosan sponges increased their ability to absorb hydrophobic agents, including antifungal agents.

As reported in detail below, Chitosan/PEG 8000 sponges, prepared in a single lyophilization step, Chitosan/PEG 6000 prepared in a single lyophilization step (1 Lyo), and Chitosan/PEG 6000 prepared using a double lyophilization method (2 Lyo) all exhibited increased degradation relative to control chitosan sponges that did not include PEG. Interestingly, the number of lyophilization steps used in preparing the sponge affected the sponge's degradation profile. Sponges prepared using a single lyophilization showed greater degradation than sponges that underwent two lyophilizations. The Chitosan/PEG 6000 1 Lyo sponges released the most amphotericin B after 1 and 6 hours of elution, while the Chitosan/PEG 8000 2 Lyo sponges eluted the most amphotericin B of all the PEG modified sponges at 3, 24, and 72 hours. Among all sponges, the 1% and 0.5% chitosan sponges released the most amphotericin B at 48, 24, and 72 hours, respectively. None of the chitosan/PEG sponges exhibited significantly lower cell numbers than the control polyurethane sponges, and all exhibited higher cell numbers than the chitosan sponges. These results indicate that adding polyethylene glycol to the chitosan sponges increased in vitro degradation, except for the Chitosan/PEG 8000 2 Lyo sponges after 4 days. All sponges released the antifungal at levels well above the amphotericin B minimum inhibitory concentration of C. Albicans; however, the sponges also retained a significant portion of the originally loaded antifungal. This in vitro study did not address long term sponge degradation.

The addition of PEG to the chitosan sponges did not cause any viability issues with normal human dermal fibroblasts, and biocompatibility of the amphotericin B eluates on normal human dermal fibroblasts will be evaluated in future studies. To ensure there is no loss of activity after release from the sponges, antifungal activity of the amphotericin B eluates will also be evaluated with C. albicans. The degradation and biocompatibility of the chitosan/PEG sponges is currently being evaluated in vivo in a rat muscle pouch model. Because of toxicity concerns with antifungals, the efficacy and biocompatibility of locally delivered amphotericin B will be evaluated in vivo.

These experiments serve as a first step in identifying a local antifungal delivery system, and due to the polymicrobial nature of orthopedic wound infections, should be further evaluated with both antifungals and antibiotics. The results of these studies are useful in identifying a degradable local delivery system that can be utilized as a local antifungal therapy, adjunctive to surgical treatment, for orthopedic wounds.

Accordingly, the invention provides compositions comprising a hydrophobic antifungal agent for the treatment or prevention of a pathogen infection. Advantageously, loading of the biologically active agents into the chitosan

The chitosan-PEG compositions can be used to form wound management devices that can be loaded with antifungal agents (e.g., Amphotericin B) and/or antibacterial agents at point of care. In one example, the chitosan-PEG compositions showed elution profiles of the hydrophobic, antifungal agent Amphotericin B that were consistently above the minimum inhibitory concentration for inhibiting Candida albicans. Thus, the invention provides wound management devices whose degradation and drug elution properties can be customized to suit the needs of specific subjects, particularly subjects having a fungal infection.

### Chitosan-PEG Compositions

Chitosan is a naturally occurring linear polysaccharide composed of randomly distributed B-(1-4)-2-amino-2-D-glucosamine (deacetylated) and B-(1-4)-2-acetamido-2-D-glucoseamine (acetylated) units. Chitosan is derived from chitin, a naturally occurring polymer. Chitin is a white, hard, inelastic, nitrogenous polysaccharide isolated from fungi, mollusks, or from the exoskeletons of arthropods (e.g., crustaceans, insects). The major procedure for obtaining chitosan is the alkaline deacetylation of chitin with strong alkaline solution. Generally, the raw material is crushed, washed with water or detergent, and ground into small pieces. After grinding, the raw material is treated with alkali and acid to isolate the polymer from the raw crushed material. The polymer is then deacetylated by treatment with alkali. Chitin and chitosan differ in their degrees of deacetylation (DDA). Chitin has a degree of deacetylation of 0% while pure chitosan has a degree of deacetylation of 100%. Typically, when the degree of deacetylation is greater than about 50% the polymer is referred to as chitosan.

Chitosan is a cationic weak base that is substantially insoluble in water and organic solvents. Typically, chitosan is fairly soluble in dilute acid solutions, such as acetic, citric, oxalic, proprionic, ascorbic, hydrochloric, formic, and lactic acids, as well as other organic and inorganic acids. Chitosan's charge gives it bioadhesive properties that allow it to bind to negatively charged surfaces, such as biological tissues present at a site of trauma or negatively charged implanted devices.

Chitosan's degree of deacetylation affects it resorption. As the degree of deacetylation increases, chitosan becomes increasingly resistant to degradation. Chitosan-PEG compositions having a degree of deacetylation that is higher than 95% degrade slowly over weeks or months. In the body chitosan is degraded by lysozyme, N-acetyl-o-glucosaminidase, and lipases. Lysozyme degrades chitosan by cleaving the glycosidic bonds between the repeating chitosan units. The byproducts of chitosan degradation are saccharides and glucosamines that are gradually absorbed by the human body. Therefore, when chitosan is used for the local delivery of therapeutic or prophylactic agents, no secondary removal operation is required.

Polyethylene glycol (PEG) has been demonstrated as a solvent system for the hydrophobic fatty acid, cis-2-decenoic acid, and allows for point of care loading of the fatty acid into the chitosan sponges. Polyethylene glycol is a water soluble polymer that exhibits protein resistance and low toxicity and immunogenicity. PEG has been used to modify chitosan films, fibers, and hydrogels. Modification of chitosan with PEG can occur through blending, copolymerization, or by using PEG simply as the drug solvent. Polyethyelene glycol has also been utilized as a solvent system, either as liposomes or nanoparticles, for amphotericin B in systemic delivery.

Adding PEG to chitosan films has shown to enhance protein adsorption and cell adhesion, growth, and proliferation. In chitosan and PEG blended fibers, the amount of salicylic acid release increased with an increase in PEG proportion of the fibers. Kolhe et al. (Biomacromolecules 2003, 4, 173-180) suggested that blending chitosan with PEG provides a more efficient way to improve chitosan's ductility and exhibits well dispersed phase morphology, as compared to copolymerization.

As reported herein, chitosan-PEG compositions were prepared that can be loaded with hydrophobic agents, including antifungal agents such as Amphotericin B. The weight percentage of total polymer (i.e., comprising chitosan and PEG) is at least about 1-2%. In particular embodiments, the weight percentage of total polymer is 1%. The ratio of chitosan:PEG may range from about 1:1 to 4:1. In particular embodiments, a chitosan:PEG ratio of 1:1 is used. The molecular weight of PEG is about 6,000-10,000 g/mol. In various embodiments, the PEG used is 6,000 or 8,000 g/mol. The chitosan used has a DDA between about 61% to 85%. In certain embodiments, the chitosan used has a DDA of about 61% or 71%. In other embodiments, the final sponge formulations used chitosan with 82.46±1.679% DDA.

The chitosan-PEG compositions of the invention (e.g., solids, sponges, films, hydrogels, composites) can be loaded with one or more biologically active agents at the site of care (e.g., in a surgical suite, clinic, or physician's office, trauma site, battlefield). This property allows the clinician to tailor the antibiotics or other agents used to load the chitosan wound management device to suit the needs of a particular patient.

In one embodiment, the degree of deacetylation is adjusted to provide chitosan-PEG compositions that degrade in as little as about twenty-four, thirty-six, forty-eight, or seventy two hours or that are maintained for a longer period of time (e.g., 4, 5, 6, 7, 8, 9, 10 days). In other embodiments, chitosan-PEG compositions of the invention are maintained in the body for at least about two-six weeks or more (e.g., 2, 3, 4, 5, 6 weeks, two, three or four months). In still other embodiments, chitosan-PEG compositions of the invention enhance blood clotting in a wound or other site of trauma (hemostasis). In other embodiments, the chitosan-PEG compositions are loaded with therapeutic or prophylactic agents that are clinician selected and that are delivered over at least about 1, 2, 3, 4, 5, 6, 7, 8, 9, or 10 days or for longer periods.

As described herein, the experimental results demonstrated that blending chitosan with polyethylene glycol in sponge form significantly affected sponge material properties, amphotericin B elution, antifungal activity, and in vivo degradation in rat intramuscular tissue. However, blending did not affect in vitro cytocompatibility or in vivo biocompatibility in rat tissue. Research has been minimal on local antifungal delivery systems, and many of the local delivery systems that exist release too little antifungal or are not designed to degrade. The present results indicate that blending chitosan and PEG into sponges creates biocompatible and degradable sponges capable of point-of-care amphotericin B loading and in vitro release that perform better than previously developed chitosan sponges.

### Antimicrobial Agents

Staphylococcus aureus, staphylococcus epidermidis, Pseudomonas aeruginosa and Candida albicans are pathogens that are commonly present at musculoskeletal wound sites. S. aureus is one cause of osteomyelitis and nongonococcal bacterial arthritis, and is often associated with prosthetic joint infection. The invention provides chitosan-PEG compositions useful in treating or preventing infection in a wound, complex wound, open fraction, or other site of trauma. Any antimicrobial agent known in the art can be used in the chitosan-PEG compositions of the invention at concentrations generally used for such agents.

Amphotericin B is one of the most commonly used antifungals in clinical practice and fungal resistance to this agent has not emerged over 50 years. Amphotericin B has been studied in various systemic delivery formulations, including microemulsions, polyethylene glycol (PEG) derivative liposomes, and conjugation to numerous polymers, such as PEG. The hydrophobic antifungal has also been incorporated into a few local delivery systems, including poly(methyl methacrylate) (PMMA) bone cement, a hydroxyapatite and chitosan composite, a Pluronic® based copolymer gel, and dextran hydrogels. A drawback to these developed local antifungal delivery systems is that most are pre-loaded with antifungals and are not easily customized by the clinician.

While PMMA bone cement is commonly used for local antibiotic delivery for orthopedic infections, there is conflicting data on amphotericin B release from bone cement. Some researchers found a complete absence of amphotericin B release, while other researchers saw release of the antifungal at very low levels.

Hydroxyapatite composites, made of hydroxyapatite, plaster of Paris, and either chitosan or alginate, have also been studied as local delivery systems for amphotericin B and have shown some advantages over bone cement. However, because both the acrylic bone cement and hydroxyapatite composites maintained antifungal activity for at least one month, neither of these local delivery systems would be ideal for antifungal release in a shorter time period.

Gel formulations have also been studied for local amphotericin B release for infection control. Kim et al. developed an thermosensitive vaginal gel formulation using a complex of amphotericin B and hydroxypropyl-γ-cyclodextrin in Pluronic® based multiblock copolymers. The gel formulations at a pH of 5 released approximately 60% of the loaded amphotericin B after 48 hours and exhibited complete release after 3 days. Hudson et al. developed a hydrogel of amphotericin B conjugated to dextran aldehyde, which released 2.5-4 mg after 200 hours, retained antifungal efficacy against C. albicans and exhibited no significant tissue toxicity. A dextran acrylate hydrogel has also been developed for point of care loading of amphotericin B and killed C. albicans within two hours of contact. However, despite being characterized for point of care use, the hydrogel requires 12 hours of soaking in the amphotericin B solution and 6 days of washing, which is not a practical length of time for surgical preparations. A local topical delivery system that is capable of rapid point of care loading would allow the clinician to select the appropriate antimicrobial and dosage, based on each individual patient's needs. The present invention provides an attractive system that facilitates the delivery of amphotericin B, as well as other hydrophobic and/or hydrophilic agents.

Antimicrobial agents useful in chitosan-PEG compositions of the invention include but are not limited to antibacterials, antifungals, and antivirals. An antimicrobial agent as used herein is an agent which reduces or stabilizes the survival, growth, or proliferation of a pathogen. Antimicrobial agents include but are not limited to Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin Y Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride. In particular embodiments, a chitosan-PEG composition comprises daptomycin.

In one preferred embodiment, a chitosan-PEG composition of the invention comprises an agent that treats multidrug resistant bacteria. In one approach, linezolid may be used to treat multi-drug resistant Gram positive bacteria. Linezolid is commercially available under the trade name Zyvox (Pfizer).

In other embodiments, a chitosan-PEG composition comprises one or more of the following: Benzalkonium Chloride, Cetylpyridinium Chloride, and Chlorhexidine Digluconate. In still other embodiments, a chitosan-PEG composition comprises one or more of antimicrobials: Polyhexamethylene Biguanide, Octenidine Dihydrochloride, Mild Silver Protein, Povidone Iodine (solution or ointment), Silver Nitrate, Silver Sulfadiazine, Triclosan, Cetalkonium Chloride, Myristalkonium Chloride, Tigecycline, Lactoferrin, Quinupristin/dalfopristin, Linezolid, Dalbavancin, Doripenem, Imipenem, Meropenem, and Iclaprim.

In still other embodiments, the antimicrobial is a fatty acid (e.g., Cis-2-Decenoic Acid). Cis-2-decenoic acid has been shown to inhibit S. aureus growth and biofilm formation.

Antifungal agents useful in chitosan-PEG compositions of the invention include but are not limited to fungicidal and fungistatic agents such as, for example, benzoic acid, undecylenic alkanolamide, ciclopirox olamine, polyenes, imidazoles, allylamine, thicarbamates, amphotericin B, butylparaben, clindamycin, econaxole, fluconazole, flucytosine, griseofulvin, nystatin, voriconazole, and ketoconazole. In a particular embodiment, the antifungal is amphotericin B.

In one embodiment, the invention provides chitosan-PEG compositions comprising a combination of one or more antimicrobials and antifungals.

### Hydrophobic Agents

The chitosan-PEG composition of the invention can be loaded with hydrophobic agents and are useful for the delivery of hydrophobic agents. Hydrophobic agents useful in chitosan-PEG compositions of the invention may be any hydrophobic agent, and include antifungals, antibiotics, anticancer drugs, and steroids. Hydrophobic agents include but are not limited to voriconazole (antifungal), rifampin (antibiotic), erythromycin (antibiotic), novobiocin (antibiotic), fusidic acid (antibiotic), and paclitaxel (anticancer drug).

### Growth Factors

Growth factors are typically polypeptides or fragments thereof that support the survival, growth, or differentiation of a cell. Such agents may be used to promote wound healing. A chitosan-PEG composition described herein can be used to deliver virtually any growth factor known in the art. Such growth factors include but are not limited to angiopoietin, acidic fibroblast growth factors (aFGF) (GenBank Accession No. NP_149127) and basic FGF (GenBank Accession No. AAA52448), bone morphogenic protein (BMP)(GenBank Accession No. BAD92827), vascular endothelial growth factor (YEGF) (GenBank Accession No. AAA35789 or NP_001020539), epidermal growth factor (EGF)(GenBank Accession No. NP_001954), transforming growth factor a (TGF-a) (GenBank Accession No. NP_003227) and transforming growth factor (3 (TFG-(3) (GenBank Accession No. 1109243A), platelet-derived endothelial cell growth factor (PD-ECGF)(GenBank Accession No. NP_001944), platelet-derived growth factor (PDGF)(GenBank Accession No. 1109245A), tumor necrosis factor a (TNF-a)(GenBank Accession No. CAA26669), hepatocyte growth factor (HGF)(GenBank Accession No. BAA14348), insulin like growth factor (IGF)(GenBank Accession No. P08833), erythropoietin (GenBank Accession No. P01588), colony stimulating factor (CSF), macrophage-CSF (M-CSF)(GenBank Accession No. AAB59527), granulocyte/macrophage CSF (GM-CSF) (GenBank Accession No. NP_000749) and nitric oxide synthase (NOS)(GenBank Accession No. AAA36365). In one preferred embodiment, the growth factor is BMP.

### Analgesics

Chitosan-PEG compositions of the invention can be used for the delivery of one or more agents that ameliorate pain, such agents include but are not limited to opioid analgesics (e.g. morphine, hydromorphone, oxymorphone, levorphanol, levallorphan, methadone, meperidine, fentanyl, codeine, dihydrocodeine, oxycodone, hydrocodone, propoxyphene, nalmefene, nalorphine, naloxone, naltrexone, buprenorphine, butorphanol, nalbuphine or pentazocine; a nonsteroidal antiinflammatory drug (NSAID) (e.g., aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, or a pharmaceutically acceptable salt thereof; a barbiturate sedative, e.g. amobarbital, aprobarbital, butabarbital, butabital, mephobarbital, metharbital, methohexital, pentobarbital, phenobartital, secobarbital, talbutal, theamylal or thiopental or a pharmaceutically acceptable salt thereof; a COX-2 inhibitor (e.g. celecoxib, rofecoxib or valdecoxib).

### Anti-Thrombotic Agents

Chitosan-PEG compositions of the invention are also useful for inhibiting, reducing or ameliorating clot formation. In one embodiment, a chitosan-PEG composition contains one or more anti-thrombotids (e.g., thrombin, fibrinogen, cumidin, heparin).

### Anti-Inflammatory Agents

In other embodiments, a chitosan-PEG composition is used to deliver an anti-inflammatory agent. Such anti-inflammatory agents include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

### Delivery of Agents Via Chitosan-PEG Compositions

The invention provides a simple means for delivering biologically active agents (e.g., small compounds, nucleic acid molecules, polypeptides) using a chitosan-PEG composition. The chitosan-PEG composition is delivered to a subject and the biologically active agent is eluted from the composition in situ. The chitosan-PEG composition is capable of delivering a therapeutic for the treatment of a disease or disorder that requires controlled and/or localized drug delivery over some period of time (e.g., 1, 3, 5, 7 days; 2, 3, 4 weeks; 1, 2, 3, 6, 12 months). Desirably, the chitosan-PEG composition comprises an effective amount of one or more antibiotics (e.g., amikacin, daptomycin, vancomycin), growth factors that promote wound healing, small molecules, hemostatic agents (e.g., thrombin and/or fibrinogen), anti-thrombotics (e.g., heparin), or cartilage or bone repair agents. The chitosan-PEG compositions are administered in the form of solids, sponges, films, hydrogels, or composites (e.g., sponge fragments in a hydrogel matrix).

Preferably, the chitosan-PEG composition comprises at least about 1 μg, 25 μg, 50 μg, 100 μg, 250 μg, 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antimicrobial agent). In another embodiment, the composition releases at least about 1 μg, 25 μg, 50 μg, 100 μg, 250 μg, 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antimicrobial agent) over the course of at least about 1, 2, 3, 4, 5, 6, 7, 8, 9, 10, 14, 21, 28, or 35 days. In still another embodiment, the composition comprises at least about 1 jag, 25 lag, 50 jag, 100 μg, 250 μg, 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antimicrobial agent) per cm³.

### Wound Healing Devices

The present invention provides wound healing devices that employ a chitosan-PEG composition. The wound healing devices may be configured by forming the chitosan-PEG composition into a shape and size sufficient to accommodate the wound being treated. If desired, the wound healing device comprises chitosan fibers. Wound healing devices are desirably produced in whatever shape and size is necessary to provide optimum treatment to the wound. These devices can be produced in forms that include, but are not limited to, plugs, meshes, strips, sutures, dressings, or any other form able to accommodate and assist in the repair of a wound. The damaged portions of the patient that may be treated with devices made of the chitosan-PEG composition of the present invention include, but are not limited to, bone, cartilage, skin, muscle and other tissues (nerve, brain, spinal cord, heart, lung). Other similar devices are administered to assist in the treatment repair and remodeling of a damaged tissue, bone, or cartilage. For some applications, it is desirable for the device to be incorporated into an existing tissue to facilitate wound repair. For other applications, it is desirable for the device to degrade over the course of days, weeks, or months. Such degradation may be advantageously tailored to suit the needs of a particular subject using the methods described herein. The elution and/or degradation profile of a chitosan-PEG composition (e.g., film, sponge) can be altered as described herein by modulating the following variables: degree of deacetylation, neutralization solution, solvent make-up, and chitosan weight %, and/or crystallinity.

In particular embodiments, the chitosan can be electrospun into fibers. Such methods are known in the art.

Crystallinity indicates the degree of structural order in a compound. Polymers such as chitosan are either amorphous or semicrystalline. Chitosan's crystallinity, like other polymers, depends on its type, number, and regularity of polymer-chain, side group chemistry, the degree of matrix packing or density, and crosslinking. The crystallinity of chitosan or its products can be controlled or altered during manufacture through its molecular weight, degree of deacetylation, and crosslinking to affect thermal properties, such as melting point, and physical-mechanical properties, such as tensile strength, Young's modulus, swelling and degradation.

Crosslinking is the process which links polymer chains together. In chitosan, crosslinking induces a three-dimensional matrix of interconnected, linear, polymeric chains. The degree or extent of crosslinking depends on the crosslinking agent. Exemplary crosslinking agents include sodium tripolyphosphate, ethylene glycol diglycidyl ether, ethylene oxide, glutaraldehyde, epichlorohydrin, diisocyanate, and genipin. Crosslinking can also be accomplished using microwave or ultraviolet exposure.

Chitosan's properties can also be altered by modulating the degree of deacetylation. In one embodiment, the degree of deacetylation is adjusted between about 50-100%, wherein the bottom of the range is any integer between 50 and 99, and the top of the range is any integer between 51% and 100%. In particular embodiments, the degree of deacetylation is 51%, 55%, 60%, 61%, 65%, 70%, 71%, 73%, 74%, 75%, 76%, 77%, 78%, 79%, 80%, 85%, 90%, and 95%. In general, the higher the molecular weight, the slower the degradation of the chitosan-PEG composition.

If desired, chitosan is neutralized after acid treatment. Any base known in the art (e.g., NaOH, KOH, NH₄OH, Ca(OH)₂, Mg(OH)₂, or combinations thereof) may be used to neutralize an acid-treated chitosan-PEG composition. Preferably, a neutralization solution has a pH greater than 7.4 (e.g., 7.8, 8.0, 8.5, 9.0, 10, 11, and 12, 13, 14, 15, 16). The neutralization step is optional, and not strictly required. If desired, the chitosan is treated with water, PBS, or sterile saline following acid treatment. It may comprise 0.01-10.0 M of a base (e.g., 0.01, 0.025, 0.5, 0.75, 0.1, 0.25, 0.5, 0.75, 1.0, 1.5, 2.0, 2.5, 3, 4, 5, 6, 7, 8, 9, 10 M) (e.g., NaOH). Chitosan-PEG compositions neutralized in bases having lower molarity degrade more quickly. Chitosan-PEG compositions neutralized in bases of increased molarity degrade more slowly than those neutralized at lesser molarities. Thus, the degradation properties of chitosan can be modulated by altering the molarity of the neutralizing base.

In other embodiments, the concentration of the acidic solvent used to dissolve the chitosan is adjusted or the time period used to dissolve the chitosan is altered. For example, a 0.1%, 0.5%, 1%, 2%, 3% or 5% acid solution is used. In particular embodiments, chitosan is dissolved in acetic, citric, oxalic, proprionic, ascorbic, hydrochloric, formic, salicylic and/or lactic acids, or a combination of those. In general, acidic solvents comprising increased levels of lactic acid form chitosan-PEG compositions that degrade more quickly and also have reduced strength and durability. In various embodiments, a combination of acetic and lactic acids are used. Lactic/acetic acid combinations degrade slower and are stronger. The acetic acid sponges degrade faster and are more flexible.

In contrast, lactic acid provides more flexibility. In one approach, the ratio of lactic to acetic acid is varied from 5:1, 4:1, 3:1, 2:1, 1:1, 1:2, 1:3, 1:4, to 1:5. In one embodiment, the blended acid solvent comprises 90%/10%, 80%/20% 75%/25%, 70%/30%, 60%/40%, 50%/50%. In still other embodiments, the chitosan weight % is altered from 0.25-10.0% (e.g., 0.1, 0.2, 0.25, 0.3, 0.4, 0.5, 1, 1.25, 1.5, 1.75, 2.0, 2.5, 3.0, 3.5, 4, 5, 6, 7, 8, 9, 10%). In one embodiment, a 1 wt % chitosan solution is preferred, where a 1 wt % chitosan solution contains 1 gram of chitosan per 100 ml solution. Typically, the higher the wt %, the slower the degradation.

If desired the chitosan-PEG composition is loaded with agents and the chitosan-PEG composition is delivered to a wound to form a delivery system for the agent. Preferably, the chitosan-PEG composition contains an effective amount of a chemical or pharmaceutically active component. In one embodiment, the chitosan-PEG composition self-adheres to a site at which delivery is desired. In another embodiment, an adhesive or other adhering means may be applied to the outer edges of the chitosan-PEG composition to hold the composition in position during the delivery of the chemical or pharmaceutically active component. Such adherent means may be used alone or in combination with the self-adhering properties of chitosan. Chitosan-PEG compositions provide for the local administration of a desired amount of a therapeutic agent.

Other embodiments of the present invention include wound-healing devices configured and produced as biological fasteners, such as threads, sutures and woven sheets. Threads and sutures comprising various embodiments of the chitosan-PEG composition provide a biocompatible fastening and suturing function for temporarily treating and sealing an open wound. Additionally, the biological fasteners may include pharmacologically active agents that may assist in the healing and remodeling of the tissue within and around the wound. Advantageously, such fastening and suturing devices may be treated to degrade in vivo at a desired rate. In other embodiments, the chitosan-PEG composition is administered directly to an injured area. A chitosan-PEG composition of the invention is administered by sprinkling, packing, implanting, inserting or applying or by any other administration means to a site of trauma (e.g., open wound, open fracture, complex wound).

### Hemostatic Compositions

The invention further provides chitosan-PEG compositions in the form of a hemostatic matrix (e.g., hemostatic sponges). Such compositions are useful alone or may be used for the delivery of a therapeutic or prophylactic agent delineated herein.

Such matrices generally comprise porous compositions formed from chitosan. In general, sponges can be formed by providing a liquid solution of chitosan and polyethylene glycol capable of forming a porous three-dimensionally stable structure. In one embodiment, a chitosan solution is prepared by dissolving polyethylene glycol and deacetylated chitosan in an acidic solvent. The components are dissolved in acid (e.g., 1% acetic acid or 1% 75:25 lactic:acetic acid). Use of acetic acid (1%) showed enhanced degradation properties and sponge flexibility. The components may be dissolved in any order. However, chitosan was easier to dissolve, when it was added after PEG had been dissolved.

A sponge is formed by casting the solution in a mold to achieve a desired shape. The chitosan solution is then frozen (e.g., at −20° C.-−80° C.) and lyophilized, thereby forming a chitosan sponge. Lyophilization is conducted to reduce the liquid (e.g. water) content of the matrix to less than about 5%, 10%, 20%, 25%, 30%, 40%, 50%, 75%, 80%, 90%, 95%, or 100% by weight. If desired, a second lyophilization step is carried out. This step is strictly optional. Following one or more lyophilizations, the chitosan-PEG composition may still include some amount of water. Typically, lyophilization removes at least about 70%, 75%, 80%, 90%, 95, or 100% or the original water content of the chitosan-PEG composition. Chitosan-PEG compositions that retain some moisture may be packaged in sterile foil to maintain such moisture.

In one approach, the sponge is neutralized, for example, by treatment with a basic solution, and re-lyophilized. Even without treatment with base, it was found that the chitosan-PEG sponges are near neutral in pH. In one embodiment, the chitosan-PEG sponge is soaked in a solution of NaOH (e.g., 0.25M, 0.6M, or 1M NaOH), and washed with water until a neutral pH is reached. Optionally, the neutralized chitosan-PEG sponges can be frozen and lyophilized a second time.

The sponge matrix is stabilized structurally and remains in a highly dense and compacted state until contacted with a liquid susceptible to absorption by the matrix, for example, body fluids. For medical use, the compacted or compressed sponge is sterilized using any suitable means (e.g., radiation). The device is packaged in sterile packaging for medical use. Sponge elements or other devices of the invention may also contain one or more active therapeutic agents. For example, they include agents that promote clotting (e.g., thrombin and/or fibrinogen). Alternatively or in addition, sponge elements or other devices of the invention include antibiotics and/or growth factors that promote tissue growth and healing.

A chitosan-PEG composition is incubated with a therapeutic agent such that the agent is incorporated into the chitosan-PEG composition. This incubation is typically carried out before or during a procedure to treat a subject using methods described herein. Sponge materials of the invention will advantageously be expandable when wetted.

Preferably, the sponge has the capacity to expand at least about 10%-100% (10, 20, 30, 40, 50). In other embodiments, a sponge expands by about 200% by volume when wetted to saturation with deionized water, buffer, or an agent of the invention. Preferred sponge materials achieve rapid volume expansions (e.g., when immersed in aqueous solution). Hemostatic sponges are produced in any size required for application to a wound. In one embodiment, the expanded sponge exerts compression on surrounding tissues when implanted or delivers an active agent to the implantation site and surrounding tissue.

### Chitosan Coatings

A chitosan-PEG composition may be included in a coating material, such as a film, that is used to coat or wrap a medical device (e.g., drug delivery or other medical device). Such coatings are used, for example, for treating or preventing a pathogen infection or for drug delivery. In orthopedics, many post-surgical infections are associated with implant materials. Patients receiving an orthopedic implants have an infection risk of about 5% for total joint replacements. Bacteria are passively adsorbed on bio material surfaces after implantation. The fundamental pathogenic mechanism in biomaterial-centered sepsis is microbial colonization of the biomaterials followed by adjacent damaged tissues. Patients that suffer from such infections often require the removal and replacement of the implant to eradicate the infection.

To treat or prevent an implant-associated infection a chitosan-PEG composition of the invention is applied to the medical device (e.g., implant). The chitosan-PEG composition provides for release of a therapeutic or prophylactic agent from the device. Such agents advantageously reduce the risk of infection associated with conventional implants. Such coatings can be applied to any medical device known in the art, including, but not limited to orthopedic devices (e.g., for joint implants, fracture repairs, spinal implants, screws, rods, plates); surgical devices (e.g., sutures, staples, anastomosis devices, vertebral disks, bone pins, suture anchors, hemostatic barriers, clamps, screws, plates, clips, vascular implants, tissue adhesives and sealants, tissue scaffolds); wound management devices; drug-delivering vascular stents (e.g., a balloon-expanded stents); other vascular devices (e.g., grafts, catheters, valves, artificial hearts, heart assist devices); implantable defibrillators; blood oxygenator devices (e.g., tubing, membranes); membranes; biosensors; shunts for hydrocephalus; endoscopic devices; infection control devices; dental devices (e.g., dental implants, fracture repair devices), urological devices (e.g., penile, sphincter, urethral, bladder and renal devices, and catheters); colostomy bag attachment devices; ophthalmic devices (e.g. intraocular coils/screws); glaucoma drain shunts; synthetic prostheses (e.g., breast); intraocular lenses; respiratory, peripheral cardiovascular, spinal, neurological, dental, ear/nose/throat (e.g., ear drainage tubes); renal devices; and dialysis (e.g., tubing, membranes, grafts), urinary catheters, intravenous catheters, small diameter grafts, vascular grafts, artificial lung catheters, atrial septal defect closures, electro-stimulation leads for cardiac rhythm management (e.g., pacer leads), glucose sensors (long-term and short-term), degradable coronary stents (e.g., degradable, non-degradable, peripheral), blood pressure and stent graft catheters, birth control devices, prostate cancer implants, bone repair/augmentation devices, breast implants, cartilage repair devices, dental implants, implanted drug infusion tubes, intravitreal drug delivery devices, nerve regeneration conduits, oncological implants, electrostimulation leads, pain management implants, spinal/orthopedic repair devices, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, hemodialysis devices, catheter cuff, anastomotic closures, vascular access catheters, cardiac sensors, uterine bleeding patches, urological catheters/stents/implants, in vitro diagnostics, aneurysm exclusion devices, and neuropatches.

Examples of other suitable devices include, but are not limited to, vena cava filters, urinary dilators, endoscopic surgical tissue extractors, atherectomy catheters, clot extraction catheters, coronary guidewires, drug infusion catheters, esophageal stents, circulatory support systems, angiographic catheters, coronary and peripheral guidewires, hemodialysis catheters, neurovascular balloon catheters, tympanostomy vent tubes, cerebro-spinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, and parental feeding catheters.

It is noted that in other embodiments of the present invention, the chitosan-PEG composition of the present invention may self-adhere to the medical device or may be adhered to the device by means other than coating materials, such as adhesives, sutures, or compression. Any suitable method known in the art may be utilized to adhere the chitosan-PEG composition to a surface. For example, the chitosan-PEG composition may be adhered to the surface by pressing the chitosan-PEG composition onto the device, wrapping the device with a chitosan film, or spraying a chitosan-PEG composition onto the device.

The chitosan-PEG compositions with biocompatible surfaces may be utilized for various medical applications including, but not limited to, drug delivery devices for the controlled release of pharmacologically active agents, including wound healing devices, such as hemostatic sponges, dressings, suture material and meshes, medical device coatings/films and other biocompatible implants.

### Delivery of Chitosan-PEG Compositions

Chitosan-PEG compositions can be delivered by any method known to the skilled artisan. In one approach, a chitosan-PEG composition is locally delivered to a site of trauma in the form of a film or sponge. The film, sponge, or other wound management device can be configured to fit a wound of virtually any size. In another approach, the chitosan-PEG composition is surgically implanted at a site where promotion of healing and/or treatment or prevention of infection is required. If desired, the chitosan-PEG composition is loaded with one or more antibiotics or other biologically active agents by a clinician within the surgical suite where treatment is to be provided. This advantageously allows the chitosan-PEG composition to be loaded with a specific agent or combination of agents tailored to the needs of a particular patient at the point at which care is to be provided.

### Screening Assays

As described herein, the present invention provides for the delivery of therapeutic or prophylactic agents to wounds in vivo. The invention is based in part on the discovery that therapeutic agents can be delivered using a chitosan-PEG composition where the agents and degradation of the composition is tailored to suit the needs of a particular patient. To identify chitosan-PEG compositions having the desired degradation and elution profiles, screening may be carried out using no more than routine methods known in the art and described herein. For example, chitosan-PEG compositions are loaded with one or more therapeutic agents and such compositions are subsequently compared to untreated control compositions to identify chitosan-PEG compositions that promote healing. In another embodiment, the degradation of a chitosan-PEG composition of the invention is assayed in vivo to identify the degree of deacetylation that corresponds to a the desired degradation profile. Any number of methods are available for carrying out screening assays to identify such compositions.

In one working example, candidate compounds are added at varying concentrations to a chitosan-PEG composition. The degree of infection or wound healing is then measured using standard methods as described herein. The degree of infection (e.g., number of bacteria) or wound healing in the presence of the compound is compared to the level measured in a control lacking the compound. A compound that enhances healing is considered useful in the invention; such a compound may be used, for example, as a therapeutic to prevent, delay, ameliorate, stabilize, or treat a disease described herein (e.g., tissue damage). In other embodiments, the compound prevents, delays, ameliorates, stabilizes, or treats a disease or disorder described herein. Such therapeutic compounds are useful in vivo.

In another approach, chitosan-PEG compositions having varying degrees of deacetylation are incubated in vivo, added to a wound, or are contacted with a composition comprising an enzyme having chitosan-degrading activity. The length of time required for chitosan degradation is then measured using standard methods as described herein. A chitosan-PEG composition having the desired degradation profile (e.g., degrading in 3 days, 5 days, 1 week, 2 weeks, 3 weeks, 1 month, 2 months, 3 months) is considered useful in the invention; such a composition may be used, for example, as a therapeutic to prevent, delay, ameliorate, stabilize, or treat a disease described herein (e.g., tissue damage). In other embodiments, the composition prevents, delays, ameliorates, stabilizes, or treats a disease or disorder described herein. Such therapeutic compositions are useful in vivo.

The present invention provides methods of treating pathogen infections (e.g., bacterial, viral, fungal), complex wounds, open fractures, trauma, and associated diseases and/or disorders or symptoms thereof which comprise administering a therapeutically effective amount of a composition comprising chitosan and a therapeutic or prophylactic agent of a formulae herein to a subject (e.g., a mammal, such as a human). Thus, one embodiment is a method of treating a subject suffering from or susceptible to an infection, trauma, wound, open fracture, or related disease or disorder that requires targeting of a therapeutic composition to a site. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for an infection, in need of healing, having a trauma, wound, open fracture, or related disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The agents herein may be also used in the treatment of any other disorders in which it is desirable to promote healing or treat or prevent an infection.

In one embodiment, the invention provides a method of monitoring treatment progress. The method includes the step of determining a level of diagnostic marker (Marker) (e.g., wound healing parameters, number of bacterial cells, or any target delineated herein modulated by a compound herein, C-reactive protein, cytokine levels, or indicator thereof, etc.) or diagnostic measurement (e.g., screen, assay) in a subject suffering from or susceptible to an infection, disorder or symptoms thereof, in which the subject has been administered a therapeutic amount of a chitosan-PEG composition (e.g., a chitosan-PEG composition comprising a therapeutic or prophylactic agent) herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Test Compounds and Extracts

In general, therapeutic compounds suitable for delivery from a chitosan-PEG composition are known in the art or are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:63786382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is identified as containing a compound of interest, further fractionation of the positive lead extract is necessary to isolate chemical constituents responsible for the observed effect. Thus, the goal of the extraction, fractionation, and purification process is the careful characterization and identification of a chemical entity within the crude extract that achieves a desired biological effect. Methods of fractionation and purification of such heterogeneous extracts are known in the art.

Small molecules of the invention preferably have a molecular weight below 2,000 Daltons, more preferably between 300 and 1,000 Daltons, and most preferably between 400 and 700 Daltons. It is preferred that these small molecules are organic molecules.

### Kits

The invention provides kits that include chitosan-PEG compositions. In one embodiment, the kit includes a chitosan-PEG composition containing one or more therapeutic or prophylactic agents that prevent or treat infection (e.g., one or more antimicrobial agents) or that promote healing (e.g., growth factor, anti-inflammatory, clot promoting agent, anti-thrombotic, steroid). In other embodiments, the kit contains a therapeutic device, such as a chitosan film useful in wound healing, chitosan sponge, hydrogel, or implant/prosthetic device comprising a chitosan-PEG composition described herein. If desired, the aforementioned chitosan-PEG compositions further comprise an agent described herein.

In some embodiments, the kit comprises a sterile container which contains a chitosan-PEG composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a chitosan-PEG composition of the invention is provided together with instructions for using it in a prophylactic or therapeutic method described herein. The instructions will generally include information about the use of the composition for the treatment of a trauma, infection or related disease in a subject in need thereof. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Hydrophobic Antifungal Agents are Loaded and Released from Chitosan-PEG Sponges

Polymicrobial musculoskeletal infections, especially from invasive bacteria, fungi or biofilm pathogens, are challenging complications that often increase patient morbidity, mortality, and hospitalization costs (Murray et al., J Trauma 64:S239-251). The clinical need for degradable local antimicrobial delivery systems to fight localized bacteria, fungi, and/or biofilms with patient-tailored antimicrobial point-of-care loading has resulted in the previous development of a chitosan sponge local delivery system (Hanssen, Clin Orthop Relat Res:91-96; Noel et al., Clin Orthop Relat Res 468:2074-2080; Stinner et al., J Orthop Trauma 24:592-597). The present study was designed to determine if the blending of these chitosan sponges with polyethylene glycol would result in local drug delivery systems capable of antifungal and antibiotic release for the prevention of polymicrobial wound infections.

A set of chitosan-PEG blended sponges were made having the following features:
1:1 Chitosan:PEG in 1% acetic acid
   71% DDA chitosan from Primex
   8000 g/mol PEG
   Lyophilized twice and neutralized in 1 M NaOH
2:1 Chitosan:PEG in 1% acetic acid
   71% DDA chitosan from Primex
   8000 g/mol PEG
   Lyophilized twice and neutralized in 1 M NaOH
4:1 Chitosan:PEG in 1% acetic acid
   71% DDA chitosan from Primex
   8000 g/mol PEG
   Lyophilized twice and neutralized in 1 M NaOH

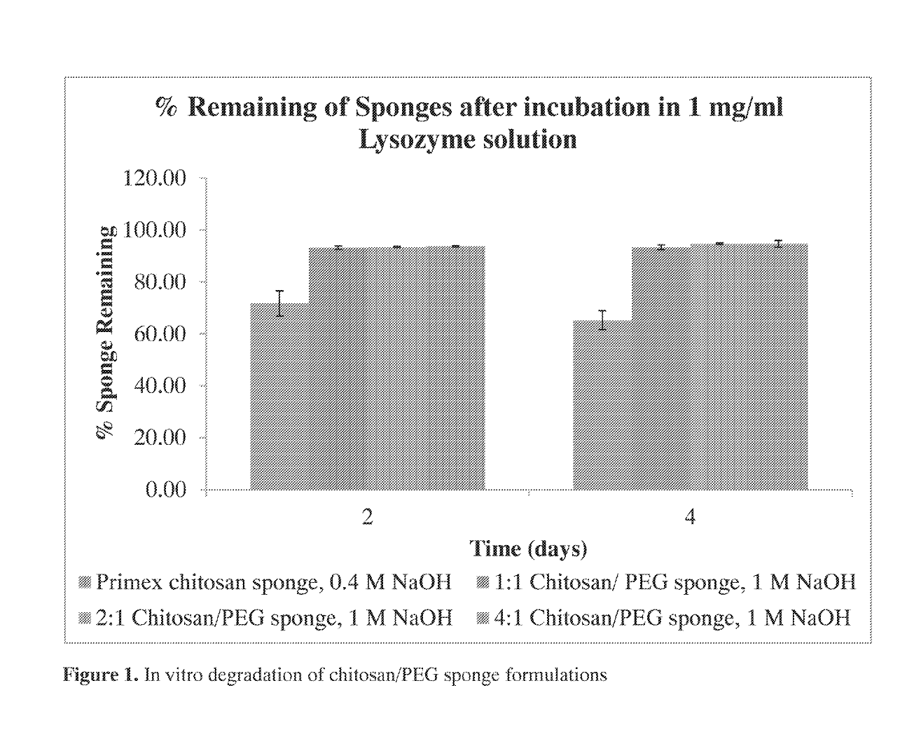

The chitosan-PEG sponge formulations were assayed in vitro for degradation, measured as % sponge remaining (mass) after incubation in a lysozyme solution (1 mg/ml; 35 ml) exchanged every 1-2 days. The chitosan-PEG sponges degraded at a slower rate, compared to a chitosan sponge (FIG. 1).

Chitosan-PEG sponges loaded with the hydrophobic antifungal agent Amphotericin B were also tested for their ability to elute Amphotericin B. Chitosan-PEG sponges were hydrated in a solution of Amphotericin B (10 ml at 1 mg/ml; Fungizone®) solubilized with sodium deoxycholate. Elution was conducted in 20 ml PBS at 37° C. with shaking throughout. Samples were taken at 1, 3, 6, 24, 48, and 72 hours and amphotericin B concentrations of the samples were measured by HPLC. For all chitosan-PEG sponges, amphotericin B concentration was higher initially at the one hour time point. However, amphotericin B concentration stabilized at 3-6 hr and remained constant until the 72 hr time point.

Another set of chitosan-PEG blended sponges (all neutralized in 0.25 M NaOH and lyophilized twice) were made having the following features:
1:1 Chitosan:PEG in 1% acetic acid, chitosan added to acid before PEG
   71% DDA chitosan from Primex
   8000 g/mol PEG
1:1 Chitosan:PEG in 1% acetic acid, PEG added to acid before chitosan
   71% DDA chitosan from Primex
   8000 g/mol PEG

From the two formulations above, it was discovered that it was preferable to add the PEG first in the acid, to let it dissolve, and then to add the chitosan. The following formulations were made with the order of addition of components as indicated.
1:1 Chitosan:PEG in 1% blended acid (75:25 lactic:acetic acid)
   71% DDA chitosan from Primex
   8000 g/mol PEG
1:1 Chitosan:PEG in 1% acetic acid
   71% DDA chitosan from Primex
   6000 g/mol PEG
1 wt % 1:1 Chitosan:PEG in 1% acetic acid
   61% DDA chitosan from Primex
   6000 or 8,000 g/mol PEG
1 wt % 1:1 Chitosan:PEG in 1% blended acid (75:25 lactic:acetic acid)
   61% DDA chitosan from Primex
   6,000, 8,000, or 10,000 g/mol PEG
1 wt % 1:1 Chitosan:PEG in 1% blended acid (75:25 lactic:acetic acid)
   71% DDA chitosan from Primex
   6,000, 8,000, or 10,000 g/mol PEG
2 wt % 1:1 Chitosan:PEG in 1% blended acid (75:25 lactic:acetic acid)
   61% DDA chitosan from Primex
   6,000 or 8,000 g/mol PEG

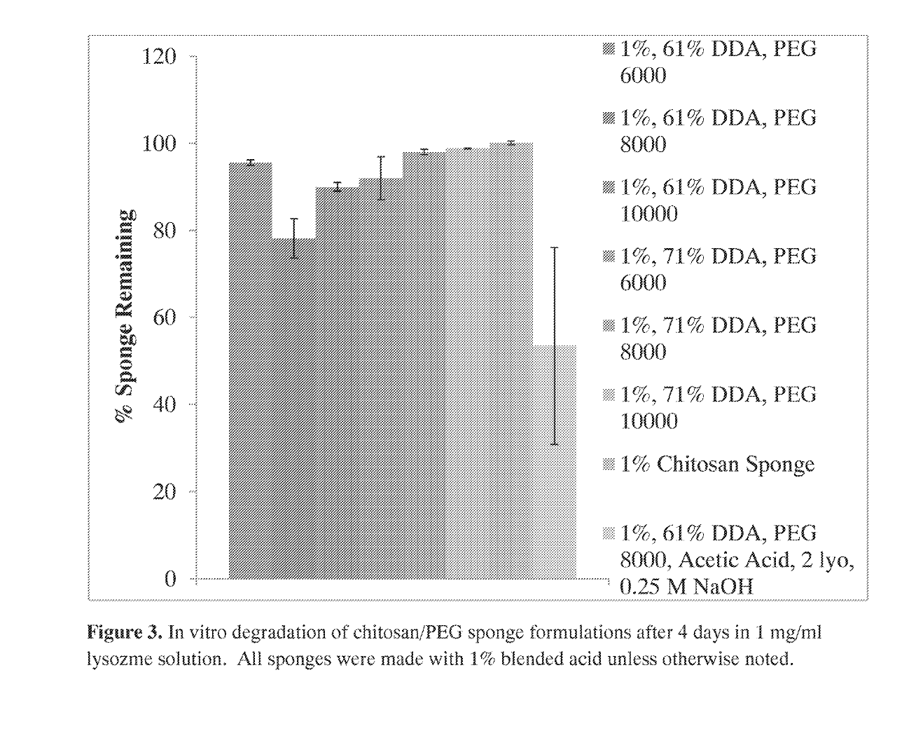

The chitosan-PEG sponge formulations were assayed in vitro for degradation, measured as % sponge remaining (mass) after incubation in a lysozyme solution (1 mg/ml; 35 ml) exchanged every 1-2 days. The chitosan-PEG sponges made with 1% blended acid (75:25 lactic:acetic acid) had degradation after 4 days similar to the chitosan sponge made without PEG (FIG. 3).

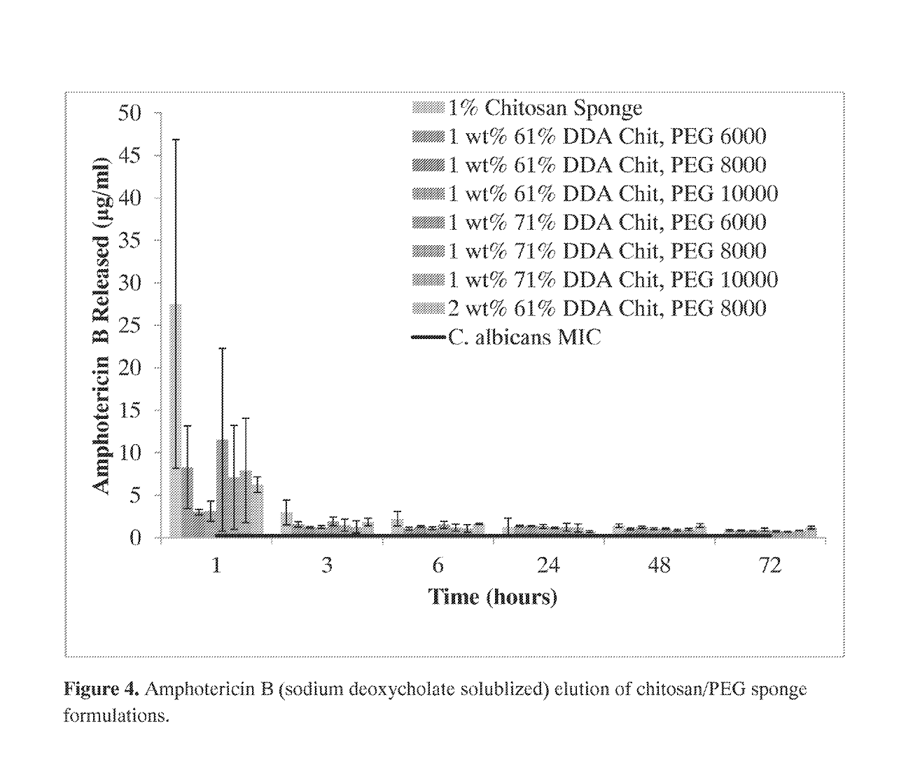

The Chitosan-PEG sponges were loaded with the hydrophobic antifungal agent Amphotericin B and tested for their ability to elute Amphotericin B. For the above sponges, the largest release of amphotericin B was observed at about 1 hour after incubation in PBS at 37° C., with subsequent release remaining constant after about 3 hours to 72 hours (FIG. 4). Importantly, amphotericin B release for the above sponges throughout the 72 hour period was above the minimum inhibitory concentration of Amphotericin B for C. albicans.

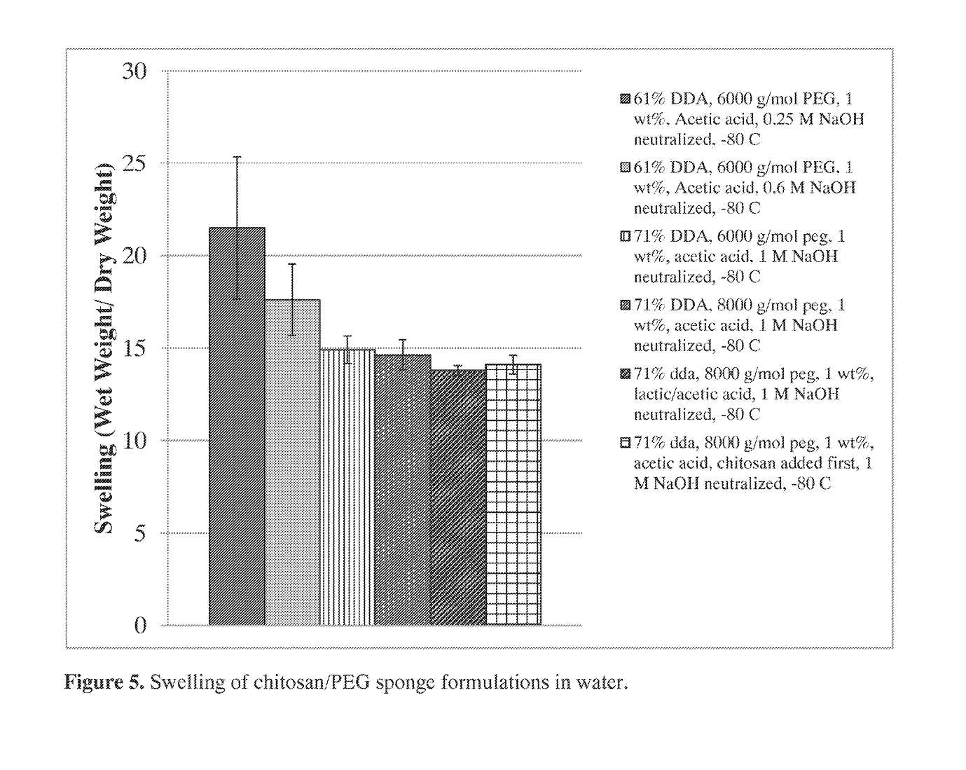

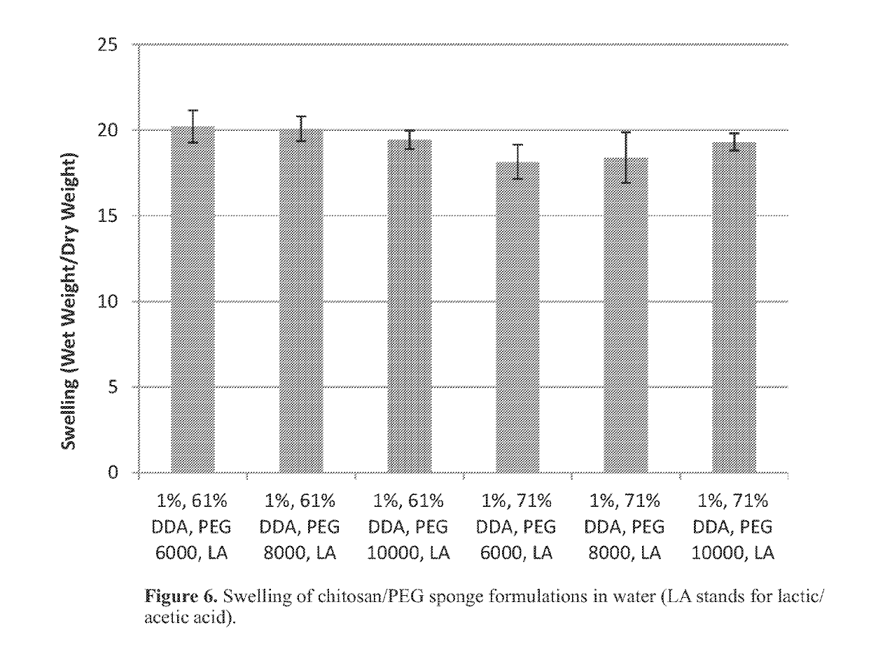

The swelling of chitosan-PEG sponge formulations in water was also tested. The chitosan-PEG sponges were able to increase their weight 15-20 fold by the absorption of water, in comparison to dry weight (FIGS. 5 and 6).

Another set of chitosan-PEG blended sponges, representing preferred chitosan-PEG sponge formulations, were made having the following features:
1 wt % 1:1 Chitosan: PEG 6000 (Modified Sponge 1)
   1× lyophilization
1 wt % 1:1 Chitosan: PEG 6000 (Modified Sponge 2)
   2× lyophilization
1 wt % 1:1 Chitosan: PEG 8000 (Modified Sponge 3)
   1× lyophilization
1 wt % 1:1 Chitosan: PEG 8000 (Modified Sponge 4)
   2× lyophilization

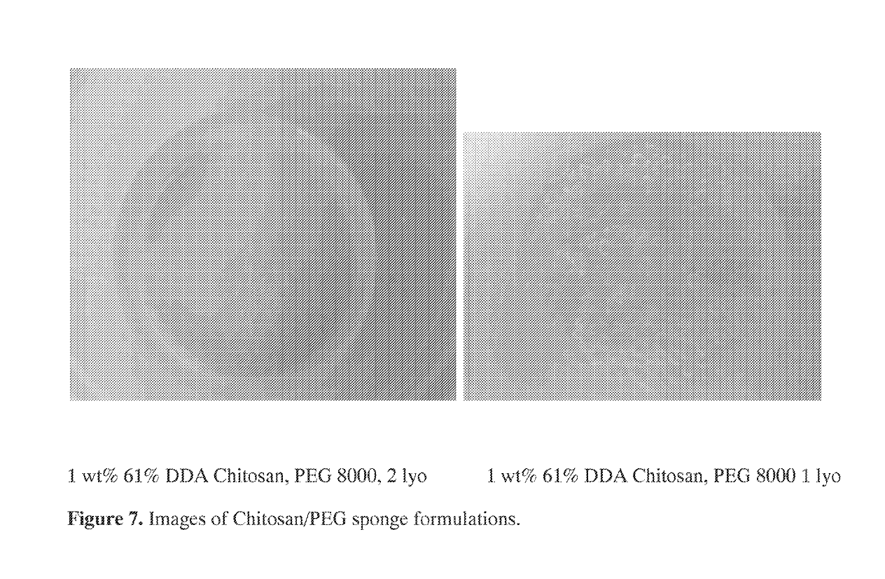

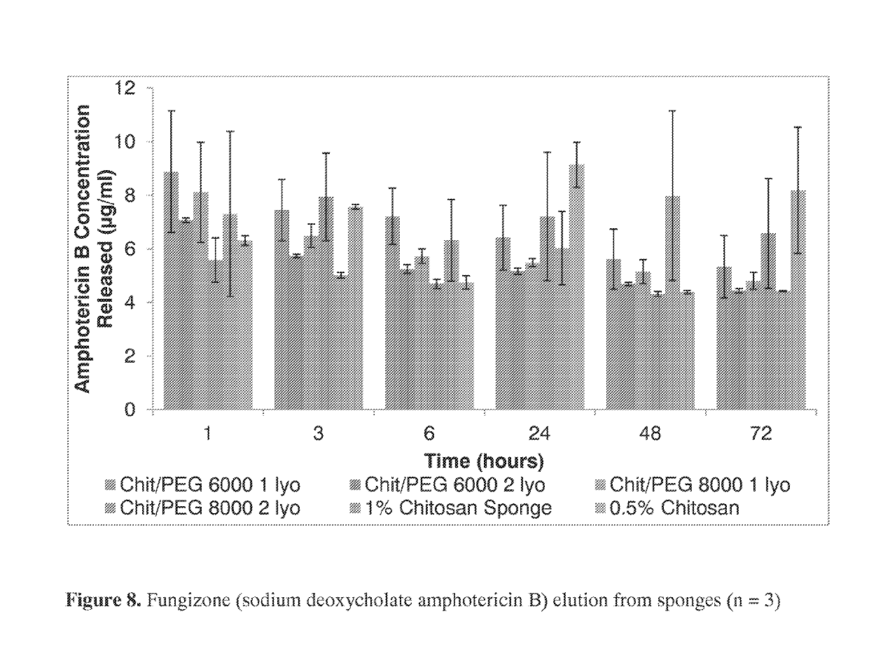

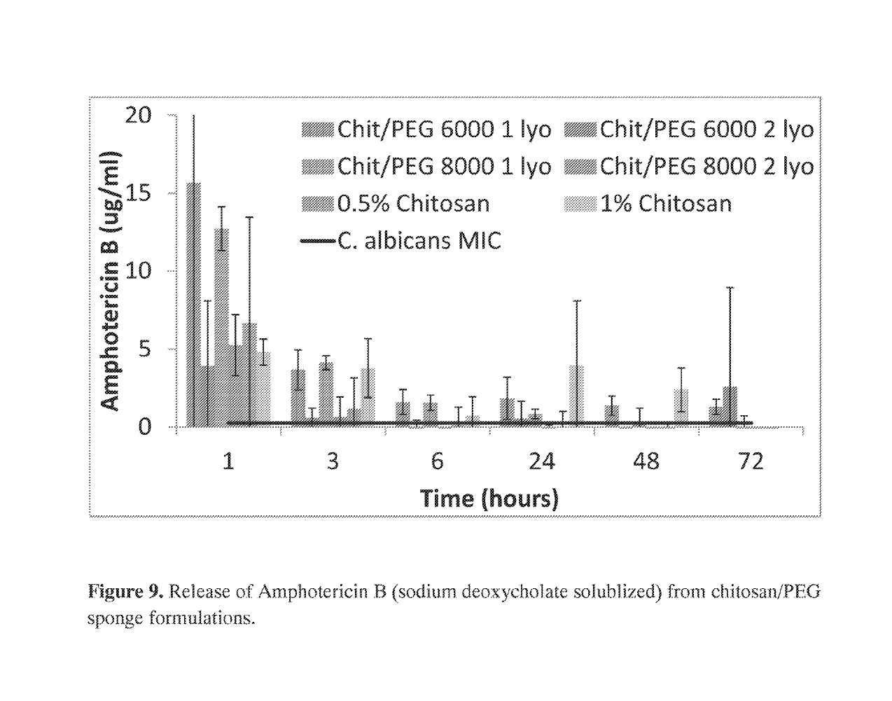

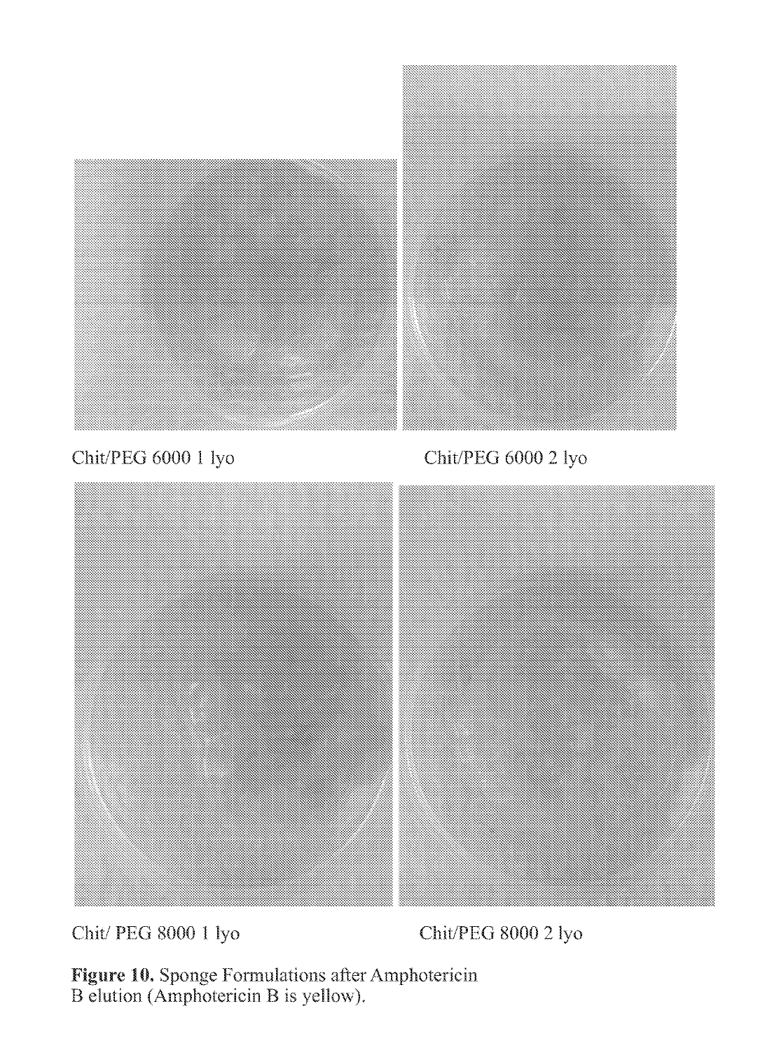

Chitosan-PEG sponge formulations made with PEG8000 using 1 or 2 lyophilization steps were similar but for smaller bubbles when 2 lyophilization steps were performed (FIG. 7). The Chitosan-PEG sponges were loaded with the hydrophobic antifungal agent Amphotericin B and tested for their ability to elute Amphotericin B. For the above sponges, a large release of amphotericin B was not observed at the 1 hour time point after the start of incubation. Release remained constant for the sponges between about 5-10 μg/ml Amphotericin B over 72 hours (FIG. 8). Once again, amphotericin B release for the sponges over the 72 hour period was above the minimum inhibitory concentration of Amphotericin B for C. albicans. Additionally, by measuring cumulative release, Amphotericin B showed a fairly steady rate of release over the course of the experiment (FIG. 9). The chitosan-PEG sponges looked similar to each other after Amphotericin B elution, which colored the sponges yellow (FIG. 10).

### Example 2: FTIR (Fourier Transform Infrared Spectroscopy) Analysis of Chitosan/PEG Blended Sponges

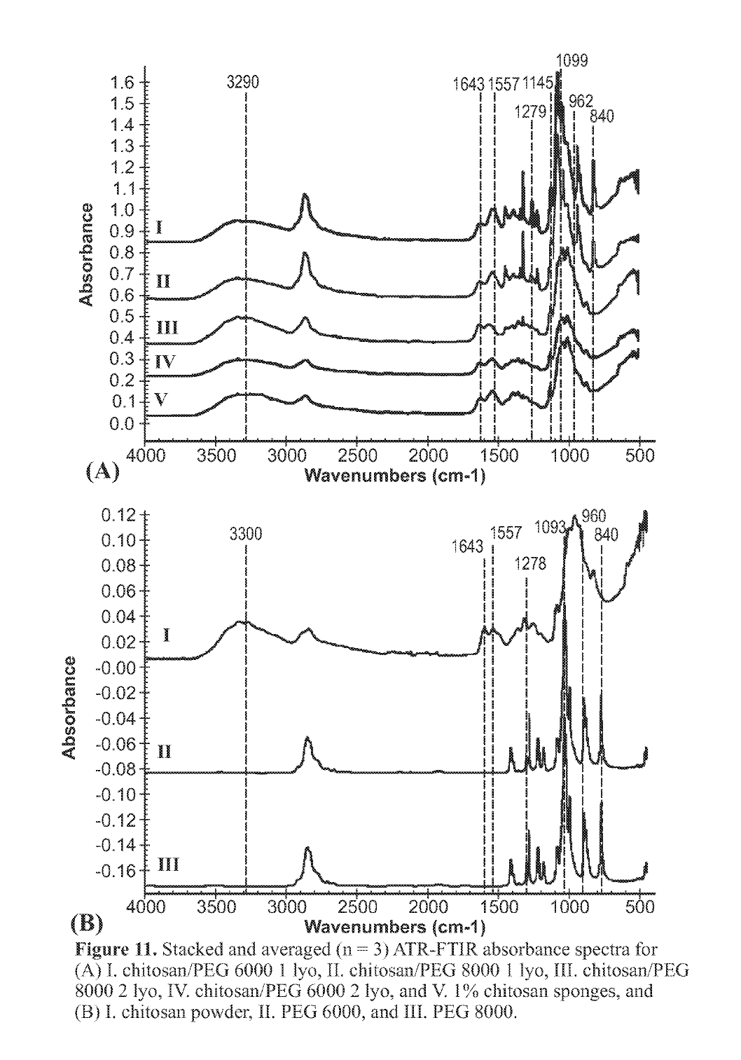

FTIR (Fourier transform infrared spectroscopy) analysis of the chitosan/PEG blended sponges and control chitosan sponges was performed (FIG. 11). The presence of PEG in the sponges was confirmed by FTIR. Characteristic peaks were seen in the 1% chitosan sponges at 3290 cm⁻¹ (O—H stretching), 1643 cm⁻¹ (amide I C═O stretching), and 1557 cm⁻¹(amide II N—H) (Wang et al. J Biomed Mater Res Part A 2008; 85(4):881-7; Kolhe et al. Biomacromolecules 2003; 4(1):173-180; He et al., Chin J Polym Sci 2009; 27(4):501-510). Characteristic C—O bending peaks were observed in PEG 6000 and 8000 at 1093 cm⁻¹, and peaks from the crystalline region of PEG were observed at 1278, 960, and 840 cm⁻¹ (Wang et al. J Biomed Mater Res Part A 2008; 85(4):881-7; Kolhe et al. Biomacromolecules 2003; 4(1):173-180).

All blended sponges exhibited the characteristic chitosan O—H stretching peaks at 3357-3360 cm⁻¹. All peaks were shifted to higher wavelengths compared to the 1% chitosan sponges. Characteristic chitosan peaks at approximately 1643 cm⁻¹ (amide I) and 1557 cm⁻¹ (amide II) cm⁻¹ were also observed in all blended sponges. All sponges and PEG also exhibited peaks at approximately 1145-1149 cm⁻¹ (C—O stretching). The C—O stretching peaks in the spectra of both 1 lyophilization blended sponges, PEG 6000, and PEG 8000 were at lower wavelength than the 1% chitosan sponge and 2 lyophilization blended sponges (He et al., Chin J Polym Sci 2009; 27(4):501-510). Both the chitosan/PEG 6000 1 lyo and 8000 1 lyo sponges exhibited the peaks seen in PEG at 1279 and 1099 cm⁻¹, which could be from the PEG crystal structure and C—O bending, respectively. However, the blended sponges with 2 lyophilizations did not exhibit these peaks. All of the blended sponges, except for the 2 chitosan/PEG 6000 2 lyo sponge, displayed the peak from PEG at 960-962 cm⁻¹. All sponges exhibited the peak at 840-843 cm⁻¹. However, both blended sponges with 1 lyophilization and PEG had peaks with higher absorbance than the 2 lyophilization blended and 1% chitosan sponges.

Additionally, the peak shift to lower wavelength at 842 cm⁻¹ in the 2 lyophilization sponges compared to the 1% chitosan sponge, indicated a potential interaction between chitosan and PEG. The blended sponges did not demonstrate a disappearance or shift of the amide I band to lower wavelength that has been reported in previous research, but all blended sponges, except for the 6000 2 lyo sponge, exhibited decreased amide I peak absorbance (Wang et al. J Biomed Mater Res Part A 2008; 85(4):881-7; Kolhe et al. Biomacromolecules 2003; 4(1):173-180). The chitosan/PEG 1 lyo sponges also demonstrated a shift of the amide II N—H peak to lower wavelength and increased absorbance compared to the chitosan sponge, which has also been confirmed by previous studies (Wang et al. J Biomed Mater Res Part A 2008; 85(4):881-7; Kolhe et al. Biomacromolecules 2003; 4(1):173-180).

### Example 3: X-Ray Diffraction (XRD) Analysis of Chitosan/PEG Blended Sponges

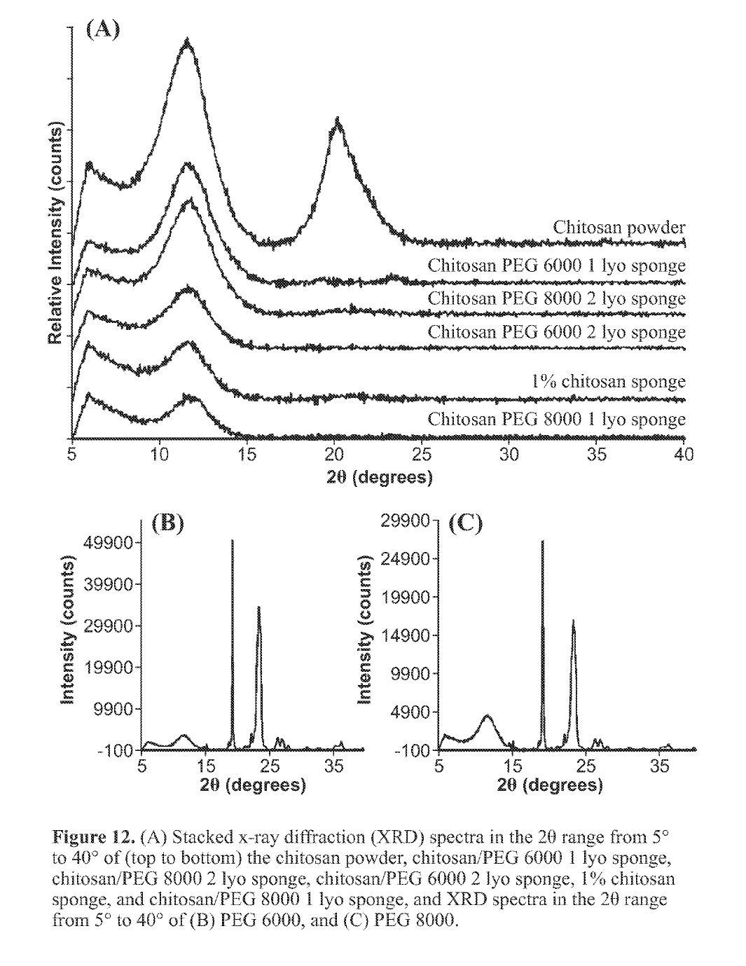

X-ray diffraction results revealed a difference between the chitosan powder and all sponge types (FIG. 12A). Analysis of the XRD spectra of the chitosan powder indicated the presence of anhydrous chitosan crystal structure (Ogawa et al., Agric Biol Chem 1991; 55(9):2375-2379). The chitosan powder exhibited the typical crystalline peak at approximately 20°, but this peak disappeared upon manufacturing the powder into sponges, both in the control chitosan sponges or the chitosan/PEG blended sponges. After chitosan dissolution and lyophilization, the crystalline peak at 20° disappeared and the 10° peak decreased and became broader, suggesting a large loss in helical crystalline forms (Zhang et al., Carbohydr Res 2005; 340(11):1914-7). Additionally, the peak at approximately 12° in the chitosan powder decreased upon sponge manufacturing, and the 1% chitosan, chitosan/PEG 6000 2 lyo, and chitosan/PEG 8000 1 lyo sponges displayed the smaller peaks. A very small peak can be seen in the chitosan/PEG 6000 1 lyo sponge at 24°, which is a contribution from the crystalline peak of PEG 6000 (seen in FIG. 12B). The increased 10° peak in both the chitosan/PEG 6000 1 lyo and 8000 2 lyo sponges indicate these sponges exhibited increased crystallinity compared to the other blended and chitosan sponges. The chitosan/PEG 6000 2 lyo and 8000 1 lyo sponges only showed a slightly reduced 10° peak from the chitosan sponge, which is similar to previously reported research on chitosan/PEG blended fibers7 and films16. Similar to the chitosan/PEG 6000 2 lyo, 8000 1lyo, and 8000 2 lyo sponges, Wang et al. also found chitosan/PEG blended fibers to be absent of PEG crystalline peaks in XRD spectra (Wang et al. J Biomed Mater Res Part A 2008; 85(4):881-7).

### Example 4: Scanning Electron Microscopy (SEM) Analysis of Chitosan/PEG Blended Sponges

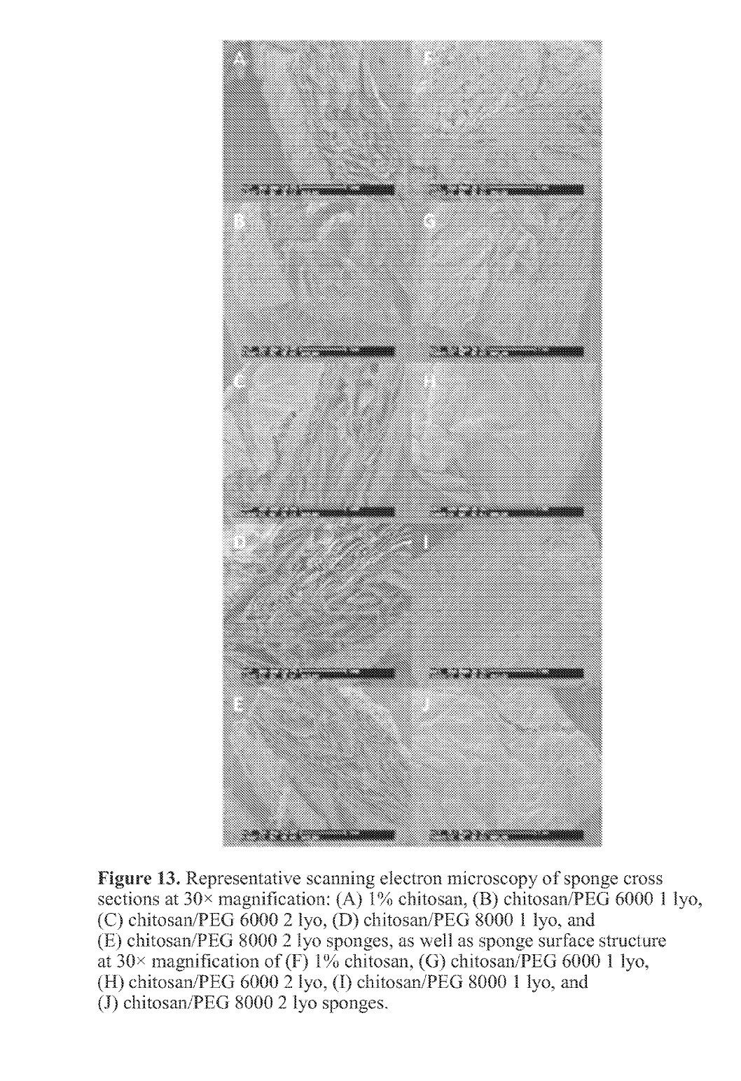

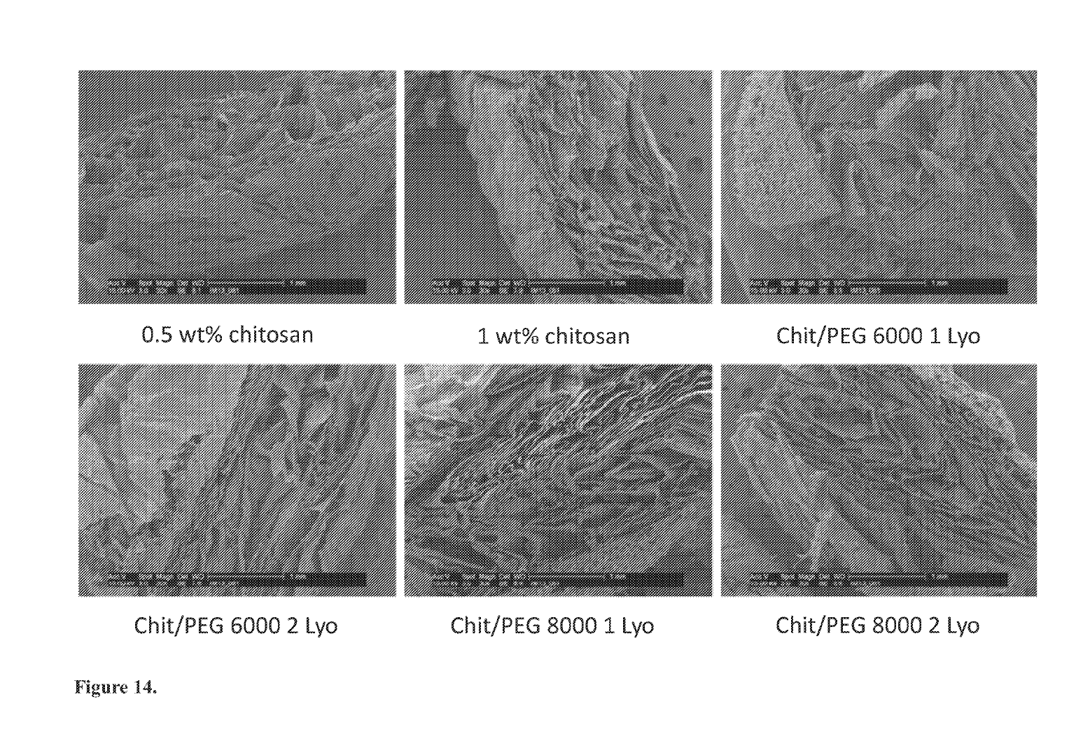

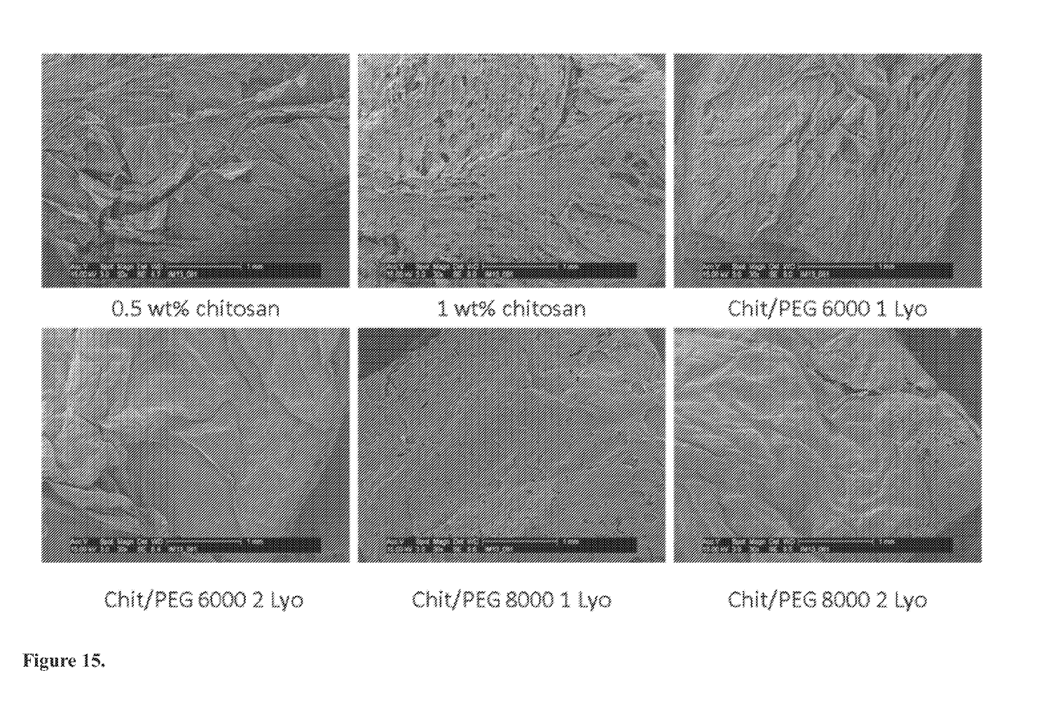

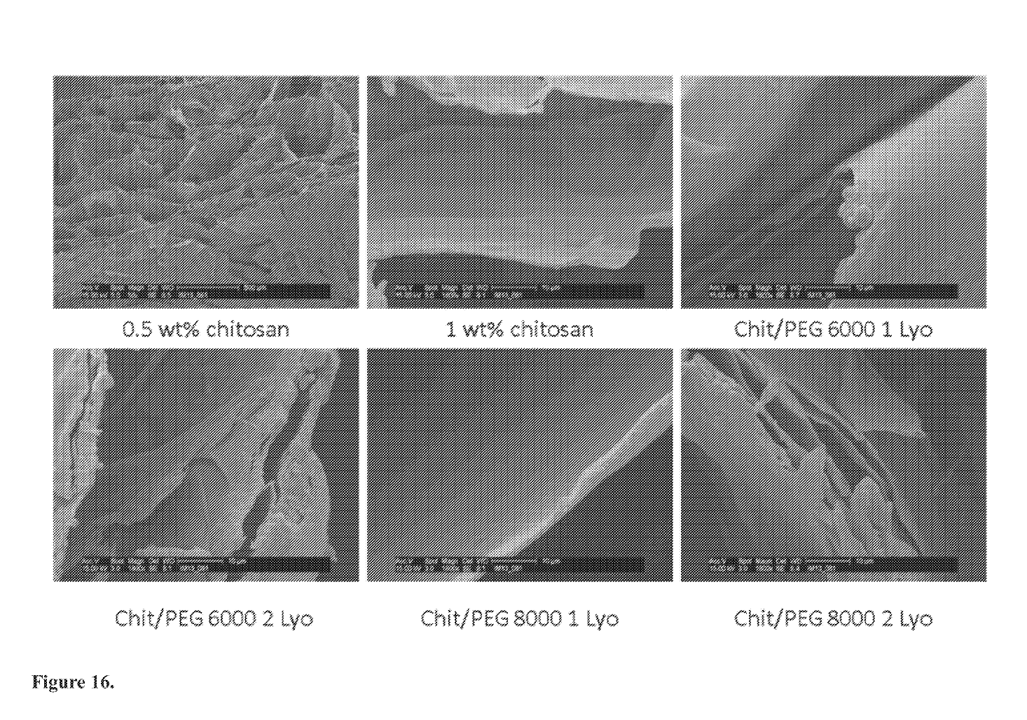

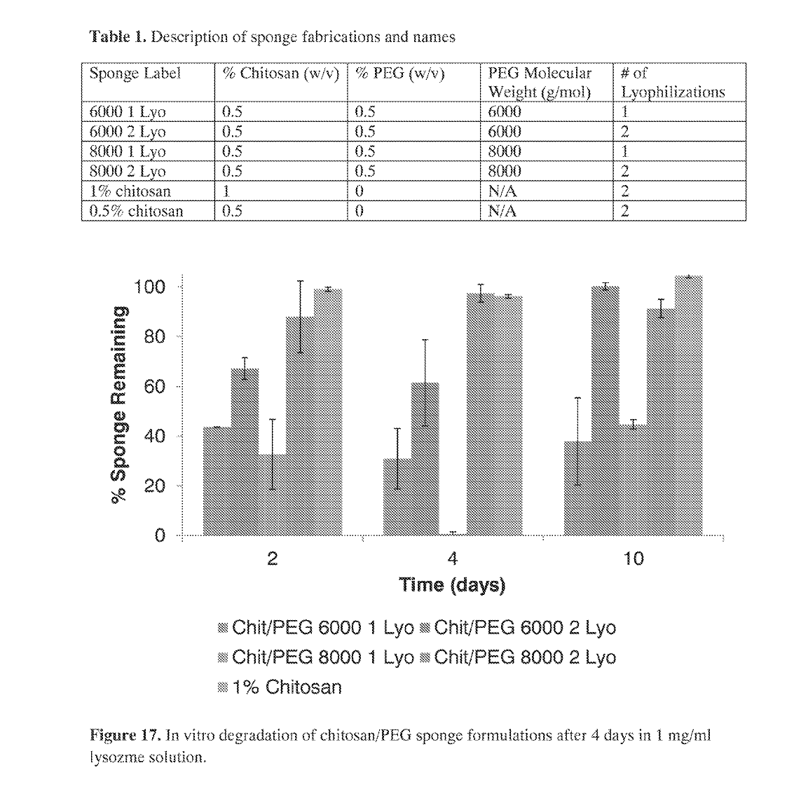

Scanning Electron Microscopy (SEM) was performed on chitosan/PEG sponge samples Morphological differences were seen in surface SEM images. However, the cross-sections of all sponges appeared similar in SEM photomicrographs (FIG. 13A-13E). However, the surface of the 1% chitosan sponge appears to have the greatest surface roughness, with the chitosan/PEG 6000 1 lyo and chitosan/PEG 8000 1 lyo sponges exhibiting a slight decrease in surface roughness (FIG. 13F-13J). In the chitosan/PEG 8000 2 lyo sponge, only a few visible pores can be seen, while none can be seen in the chitosan/PEG 6000 1 lyo sponge. The smoother appearance of the chitosan and PEG blended sponges, as compared to the 1% chitosan sponges, may indicate the compatibility of two polymers in a blend. Additional scanning electron micrographs of the sponges are provided at FIGS. 14-16. The chitosan/PEG sponges' morphologies agrees with previously reported research that chitosan/PEG blended films (Zhang et al., Biomaterials 2002; 23(13):2641-8) and fibers (Wang et al., J Biomed Mater Res Part A 2008; 85(4):881-7) exhibited smooth and nonporous surfaces (Zhang et al., Biomaterials 2002; 23(13):2641-8) and smooth and homogenous cross sections (Wang et al., J Biomed Mater Res Part A 2008; 85(4):881-7), respectively.

### Example 5: Differential Scanning Calorimetry (DSC)

Differential scanning calorimetry (DSC) was used to analyze the chitosan/PEG sponges. Thermal analysis of the chitosan powder, sponges, and chitosan/PEG blended sponges indicated that the chitosan double lyophilization and neutralization procedure decreased exothermic peak temperatures, which corresponded to amine decomposition (Guinesi et al., Thermochim Acta 2006(444):128-133). Analysis of the thermal data revealed that endothermic and exothermic peaks appeared at 56-126° C. and 313-319° C. in the sponges and powder samples (Table 1).

TABLE 1
Differential scanning calorimetry results of chitosan/PEG and chitosan
sponges, as well as chitosan, PEG 6000, and PEG 8000 powder.

| | Endothermic Peak | | | Exothermic Peak | | |
|---|---|---|---|---|---|---|
| | Temperature | Height | | Temperature | Height | |
| Sample | (° C.) | (mW/mg) | Area (J/g) | (° C.) | (mW/mg) | Area (J/g) |
| Chit/PEG | 56.03 ± 1.00^{§γ} | 1.63 ± 0.15 | −69.98 ± 4.38 | 313.73 ± 0.93*^{†} | 0.91 ± 0.02*^{†} | 93.45 ± 11.75*^{†} |
| 6000 1 Lyo | | | | | | |
| Chit/PEG | 110.7 ± 4.83 | 1.09 ± 0.11 | −191.77 ± 28.92 | 319.53 ± 1.12 | 2.34 ± 0.31 | 169.7 ± 22.02 |
| 6000 2 Lyo | | | | | | |
| Chit/PEG | 58.57 ± 0.61 | 1.62 ± 0.06 | −79.21 ± 6.28 | 315.4 ± 1.99*^{†} | 0.95 ± 0.11*^{†} | 94.82 ± 7.72*^{†} |
| 8000 1 Lyo | | | | | | |
| Chit/PEG | 122.9 ± 9.22^{γ} | 1.09 ± 0.06 | −200.7 ± 9.45 | 319.07 ± 0.15 | 2.31 ± 0.18 | 168.13 ± 2.9 |
| 8000 2 Lyo | | | | | | |
| 1% Chitosan | 126.67 ± 7.80^{§} | 1.11 ± 0.04 | −210.5 ± 12.04 | 319.17 ± 1.10 | 2.05 ± 0.07 | 160.13 ± 5.38 |
| Sponge | | | | | | |
| Chitosan | 111.6 ± 4.87 | 1.01 ± 0.11 | −181 ± 28.34 | 325.97 ± 0.06^{‡} | 3.33 ± 0.08^{‡} | 178.7 ± 8.58 |
| powder | | | | | | |
| PEG 6000 | 68.07 ± 0.55 | 6.07 ± 0.37 | −201.63 ± 9.47*^{†} | N/A | N/A | N/A |
| PEG 8000 | 67.93 ± 0.40 | 6.57 ± 0.56 | 203.23 ± 6.86*^{†} | N/A | N/A | N/A |

^{§}and^{γ}indicate p < 0.05 vs. each other,
*represents p < 0.05 versus all others except^{†},
^{‡}indicates p < 0.05 versus all others

The endothermic peak can be attributed to water loss, while the exothermic peak corresponds to the decomposition of amine units (Mecwan et al., J Biomed Mater Res Part A 2011.; Kittur et al., Carbohydr Polym 2002; 2002(49):185-193; Guinesi et al., Thermochim Acta 2006(444):128-133). Because the PEG 6000 and 8000 powder samples do not have amine units, both are lacking the exothermic peak. The chitosan powder had the highest exothermic peak temperature, but after processing the chitosan into a 1% chitosan sponge, the exothermic peak temperature decreased by 6.8° C. (p<0.001). The chitosan/PEG 2 lyo sponges also exhibited similar exothermic peak temperatures to the 1% chitosan sponge. However, the chitosan/PEG 6000 and 8000 1 lyo sponges exhibited 5.44° C. and 3.77° C. lower exothermic peak temperatures than the 1% chitosan sponge (p<0.001 and p=0.008). Additionally, the height and area of the exothermic peaks in the chitosan/PEG 1 lyo sponges also decreased significantly from the chitosan/PEG 2 lyo and 1% chitosan sponges. The shift of the exothermic peak to lower temperatures in the 1 lyo blended sponges is similar to previously reported findings that, with increasing PEG content in chitosan and PEG blended films, the exothermic peak temperature shifted to lower temperatures (He et al., Chin J Polym Sci 2009; 27(4):501-510). The chitosan/PEG 1 lyo sponges also exhibited lower endothermic peak temperatures and areas than the chitosan/PEG 2 lyo sponges, 1% chitosan sponges, and chitosan powder. The decreased endothermic peak temperatures in the chitosan/PEG 1 lyo sponges can be attributed to the melting temperature of PEG 6000 and 8000, approximately 68° C. The shift to even lower endothermic peak temperatures can be explained by increased lattice defects due to partial miscibility of the noncrystalline phase (He et al., Chin J Polym Sci 2009; 27(4):501-510; Wan et al., Biomacromolecules 2006; 7(4):1362-72).

### Example 6: In Vitro Degradation and Viscosity of Chitosan/PEG Blended Sponges

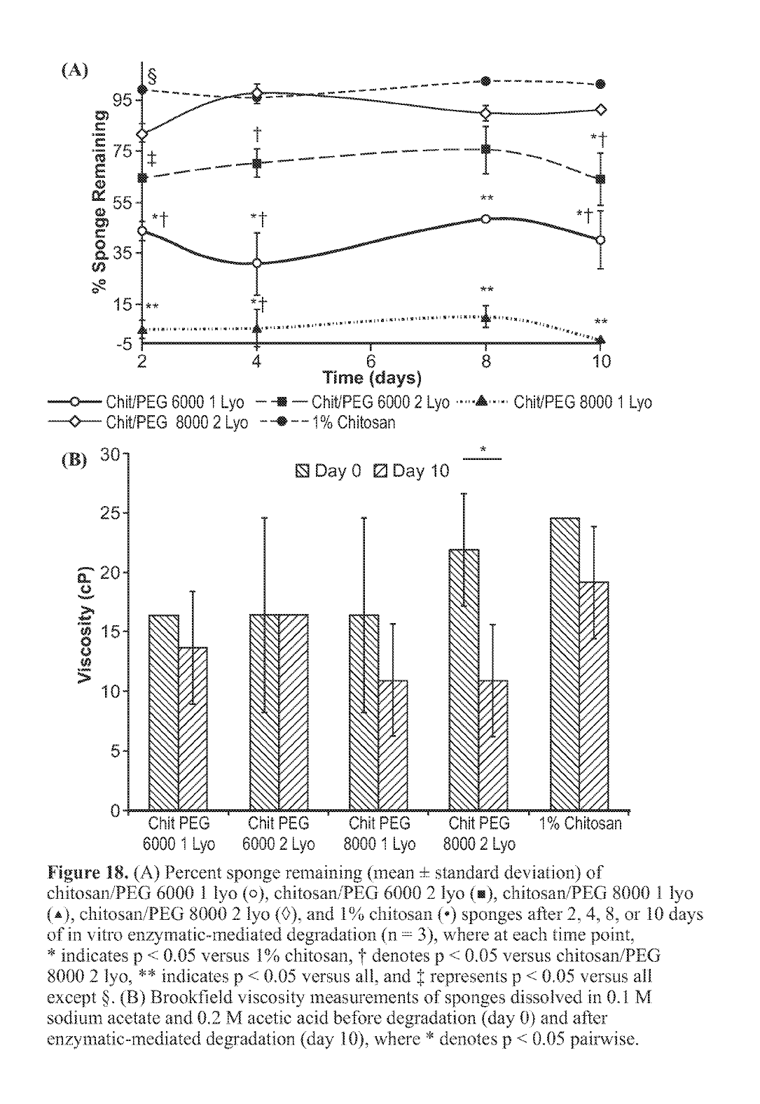

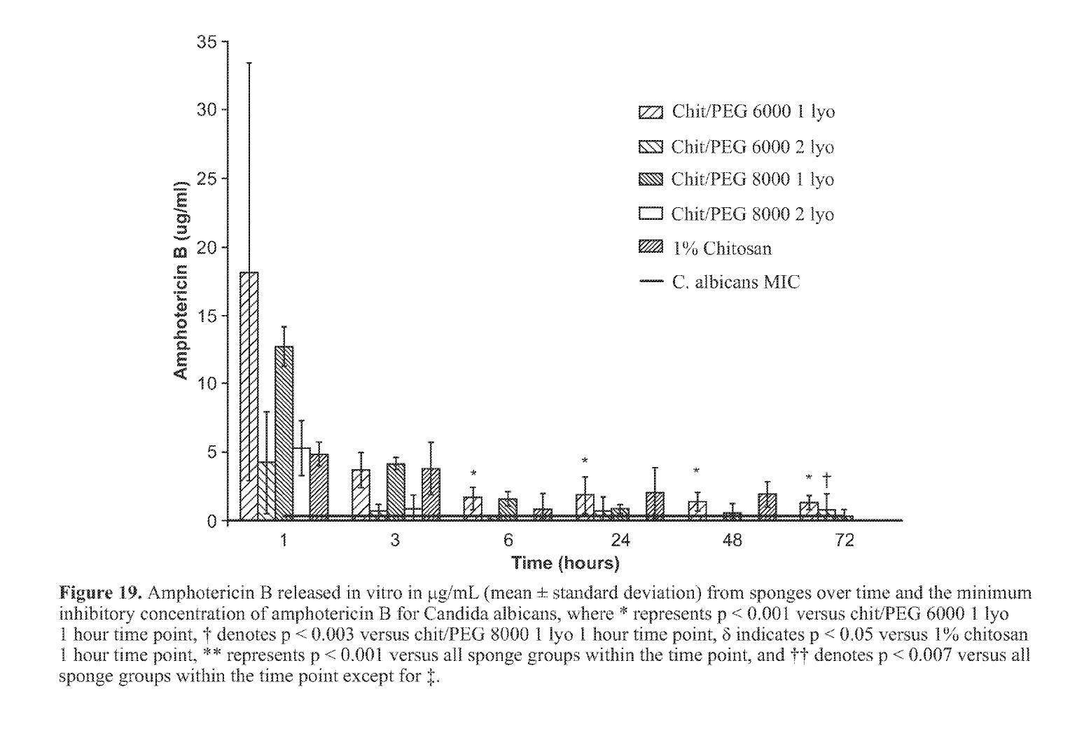

The chitosan-PEG sponge formulations were assayed in vitro for degradation by incubation in a lysozyme solution (1 mg/ml; 35 ml) exchanged every 1-2 days. In vitro sponge degradation was affected by the addition of PEG, molecular weight of PEG, and number of lyophilizations. Chitosan-PEG sponges having a lower molecular mass (PEG 6000) and/or produced with a single lyophilization step degraded at a faster rate, compared to the 1% chitosan sponge and chitosan-PEG having a higher molecular mass (PEG 8000) and two lyophilization steps (FIG. 17). The chitosan/PEG 6000 1 lyo, chitosan/PEG 6000 2 lyo, and chitosan/PEG 8000 1 lyo sponges exhibited significant differences in degradation, 58.99, 31.11, and 94.18% respectively, than the control chitosan sponge, as well as 49.37, 21.59, and 84.66% less sponge remaining than the chitosan/PEG 8000 2 lyo sponge (FIG. 18A). There was no significant decrease in remaining sponge over time for any of the chitosan/PEG sponges. As expected, the degradation of the blended chitosan/PEG sponges in this study is higher than the 7% weight loss after 8 days of lysozyme mediated degradation reported by Tanuma et al. (Carbohydr Polym 2010; 80(1):260-265) for PEG crosslinked chitosan films.

All of the sponges, except for the chitosan/PEG 6000 2 lyo sponges, exhibited a decrease in viscosity from day 0 to day 10 of degradation (FIG. 18B), although only the chitosan/PEG 8000 2 lyo sponge exhibited a statistically significant difference (p=0.014). However, the viscosity of the blended sponges did not decrease as much as the 55% reduction in specific viscosity of PEG grafted chitosan solutions in lysozyme reported in a previous study, which used 7.5 times as much lysozyme as used in this study (Hu et al., Carbohydr Polym 2005; 61(4):472-479). Adding PEG 6000 to the chitosan sponges and using only 1 lyophilization caused three out of four of the blended sponges to degrade significantly in mass. While molecular weight could not be directly measured through GPC methods, the slight decrease in viscosity after ten days of degradation demonstrates molecular weight of chitosan in the sponge may be decreasing as degradation occurs

### Example 7: In Vitro Amphotericin B Elution of Chitosan/PEG Blended Sponges

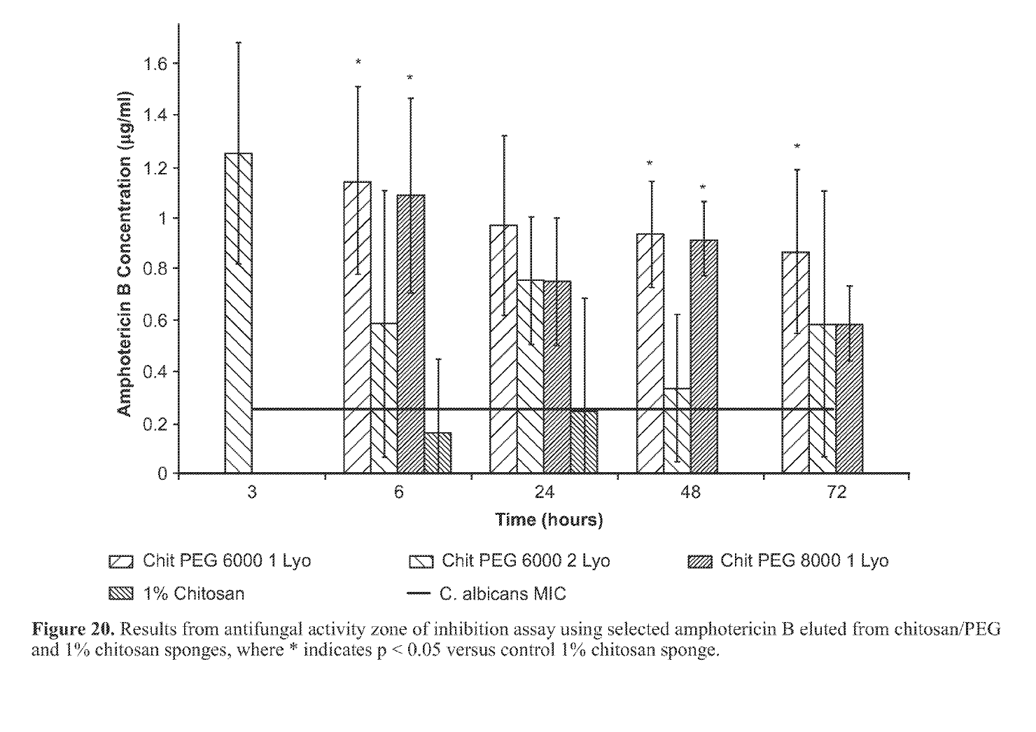

Differences in amphotericin B elution were seen in the sponges after adding PEG to the chitosan sponges and using only one lyophilization. The average concentrations of amphotericin B released from all sponges after 1 and 3 hours were all above the minimum inhibitory concentration (MIC) for Candida albicans, but this concentration was not maintained for some sponges at later time points (FIG. 19). The chitosan/PEG 6000 1 lyo sponge was the only sponge formulation to release amphotericin B at levels above the C. albicans MIC throughout the entire 72 hour elution. The chitosan/PEG 6000 and 8000 2 lyo sponges released average amphotericin B concentrations above the MIC at 72 hours, but not for all earlier time points. The cumulative percent release of loaded amphotericin B after 72 hours was determined to be 12.4±3.8, 5.3±3.6, 5.2±0.3, 5.4±2.1, and 7.7±2.2% for the chitosan/PEG 6000 1 lyo, 6000 2 lyo, 8000 2 lyo, and 1% chitosan sponges, respectively. While the chitosan/PEG 1 lyo, and 1% chitosan sponges exhibited significant differences in antifungal elution over time, the chitosan/PEG 2 lyo sponges did not release significantly different concentrations of amphotericin B over time compared to control chitosan sponges.

Although the sponges retained amphotericin B, all of the sponges released more antifungal in vitro than what has been previously reported for amphotericin B loaded bone cement, ranging from 0.05-0.45% (Kweon et al., Clin Orthop Relat Res 2011; 469(11):3002-7; Goss et al., J Arthroplasty 2007; 22(6):902-8.). As expected, the percent cumulative release from the post-loaded chitosan/PEG blended sponges was less than the 60% amphotericin B release after 2 days from a preloaded Pluronic® based multi-block copolymer gel (Kim et al., Eur J Pharm Sci 2010; 41(2):399-406). The active amphotericin B eluates from the chitosan/PEG sponges are similar to previously reported active amphotericin B eluates from PMMA bone cement (Cunningham et al., Clin Orthop Relat Res 2012; 470(10):2671-6). Although the control chitosan sponges released more cumulative loaded amphotericin B than some of the blended sponges, many of these antifungal eluates did not maintain sufficient activity against C. albicans. The antifungal activity ZOI assay results in the present study indicate that the addition of PEG to the chitosan sponges improved antifungal elution and activity over the unblended chitosan sponges.

### Example 8: Antifungal Activity In Vitro of Chitosan/PEG Blended Sponges

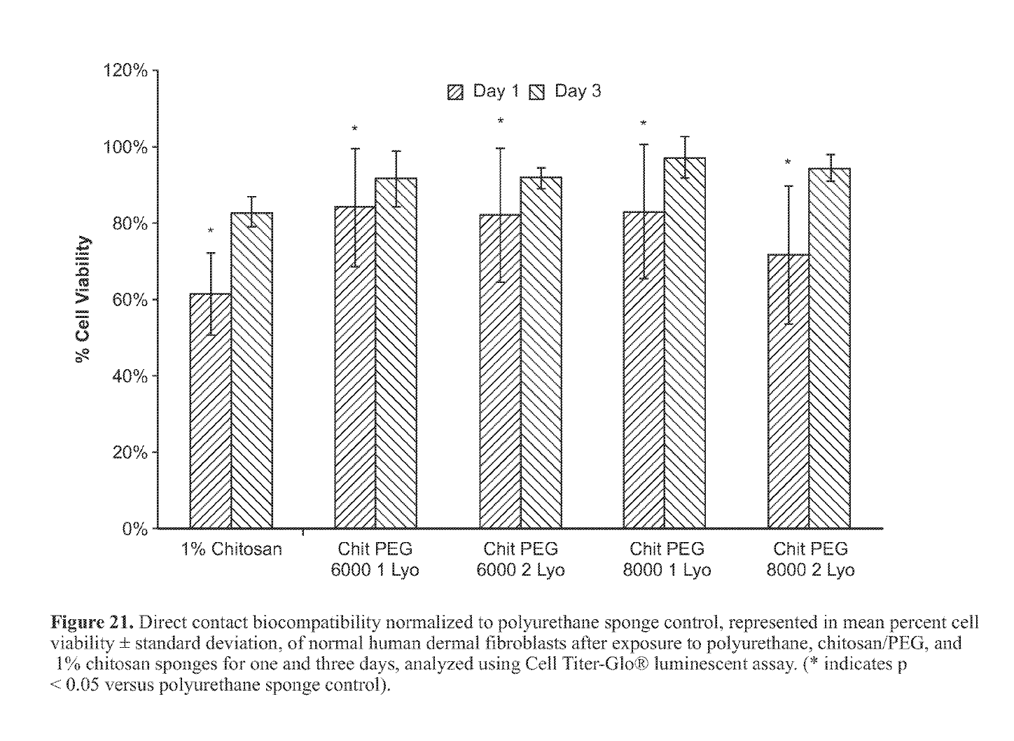

To examine fungal inhibition of Candida albicans by Amphotericin B, chitosan-PEG sponges were placed on plates of Candida albicans and the release over 72 hours examined. Elution of Amphotericin B inhibited the growth and proliferation of Candida albicans in this zone of inhibition assay. The concentration of Amphotericin B was determined using Amphotericin B standards on plates of Candida albicans; the distance of fungal inhibition was measured and correlated to the Amphotericin B concentration (FIG. 20). Consistent with fungal inhibition, it was determined that Amphotericin B release for the sponges was above the minimum inhibitory concentration of Amphotericin B for Candida albicans for up to 72 hours.

All of the selected amphotericin B eluates tested in the assay had sufficient corresponding activity on Candida albicans, except for the eluates from the 1% chitosan sponges (FIG. 20). Some of the amphotericin B eluates released from the 1% chitosan sponges that were measured at levels above the C. albicans MIC did not inhibit growth of C. albicans in the ZOI assay. In the ZOI assay, the eluates from the chitosan/PEG 6000 1 lyo and 8000 1 lyo sponges had the highest C. albicans zones of inhibition and corresponding amphotericin B concentration, and were both significantly different from the 1% chitosan sponges after 6 (p<0.006) and 48 hours (p=0.006) of elution.

### Example 9: Cytocompatibility of Chitosan/PEG Blended Sponges

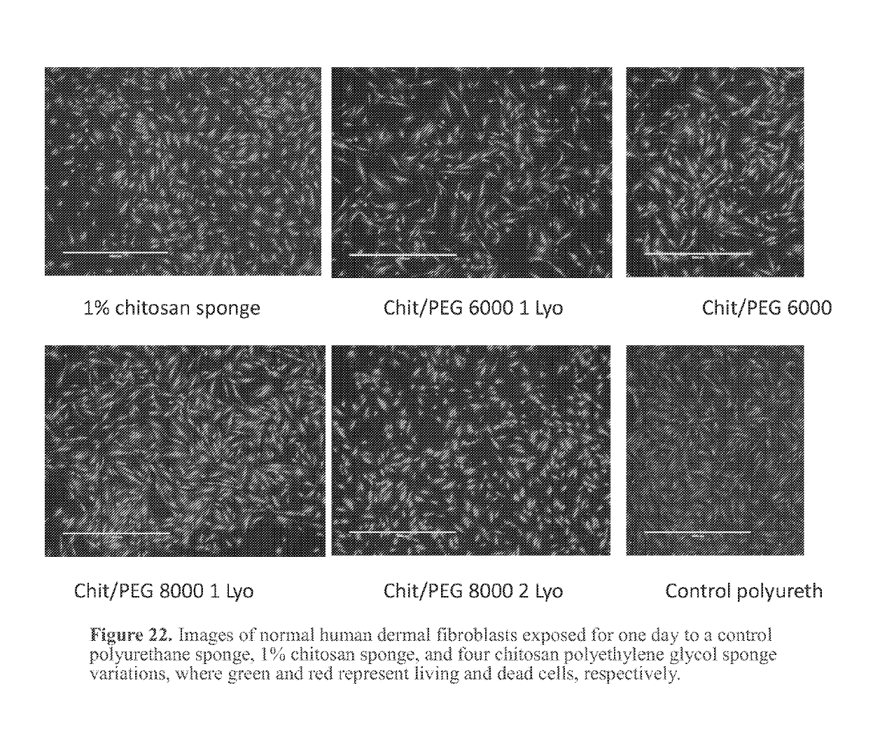

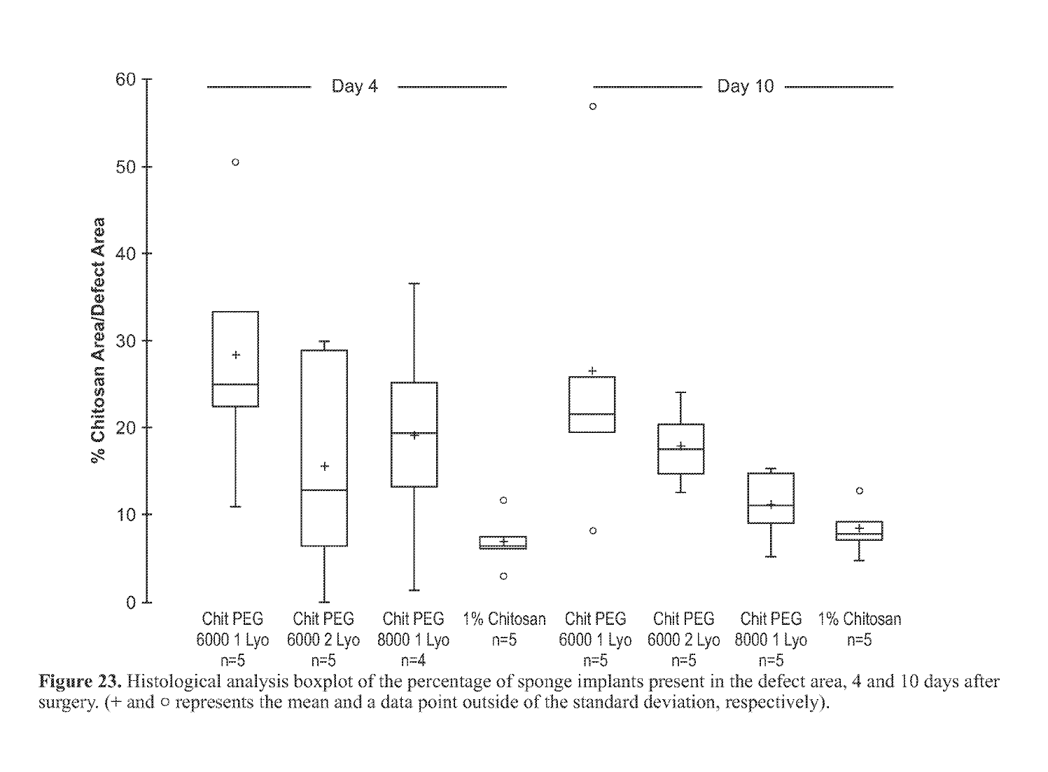

As seen in FIG. 21, each type of sponge caused significant decreases in cell viability compared to the control polyurethane sponge, (p<0.014). After 1 day, the 1% chitosan sponge and chitosan/PEG 8000 2 lyo sponges exhibited the largest decreases in percent cell viability, with 39 and 28.57% reduction in cell viability from the polyurethane control, respectively. Upon microscopic examination after one day of treatment, both the 1% chitosan and chitosan/PEG 8000 2 lyo sponges induced slight cellular morphology changes in some fibroblasts, while the other blended sponges did not cause any visible changes to the cells from the control (FIG. 22). However, after three days of treatment, there were no significant differences between any of the sponges and the polyurethane sponge control. After three days of treatment, the cellular morphology that was visible after day one for some of the sponges was no longer present. Additionally, these cytocompatibility evaluations found no sloughing, lysis, or reduction of the cell layer at either time point.

The differences in cytocompatibility found over time for all sponges in this study are also similar to previous results reported for other chitosan applications, but the chitosan/PEG 6000 1 lyo, 6000 2 lyo, and 8000 1 lyo sponges exhibited increased cytocompatibility from chitosan sponges (Parker et al. J Biomed Mater Res B Appl Biomater 2013; 101(1):110-23; Silva et al., Biomacromolecules 2008; 9(10):2764-74). Previous research has shown that adding PEG to chitosan films enhanced protein adsorption and cell adhesion, growth, and proliferation (Zhang et al., Biomaterials 2002; 23(13):2641-8). The chitosan/PEG 8000 2 lyo sponge's slight decrease in cell viability after one day of treatment indicates there may be more similarities between the chitosan/PEG 8000 2 lyo sponge and control chitosan sponge than the other blended sponges.

### Example 10: In Vivo Degradation and Biocompatibility of Chitosan/PEG Blended Sponges

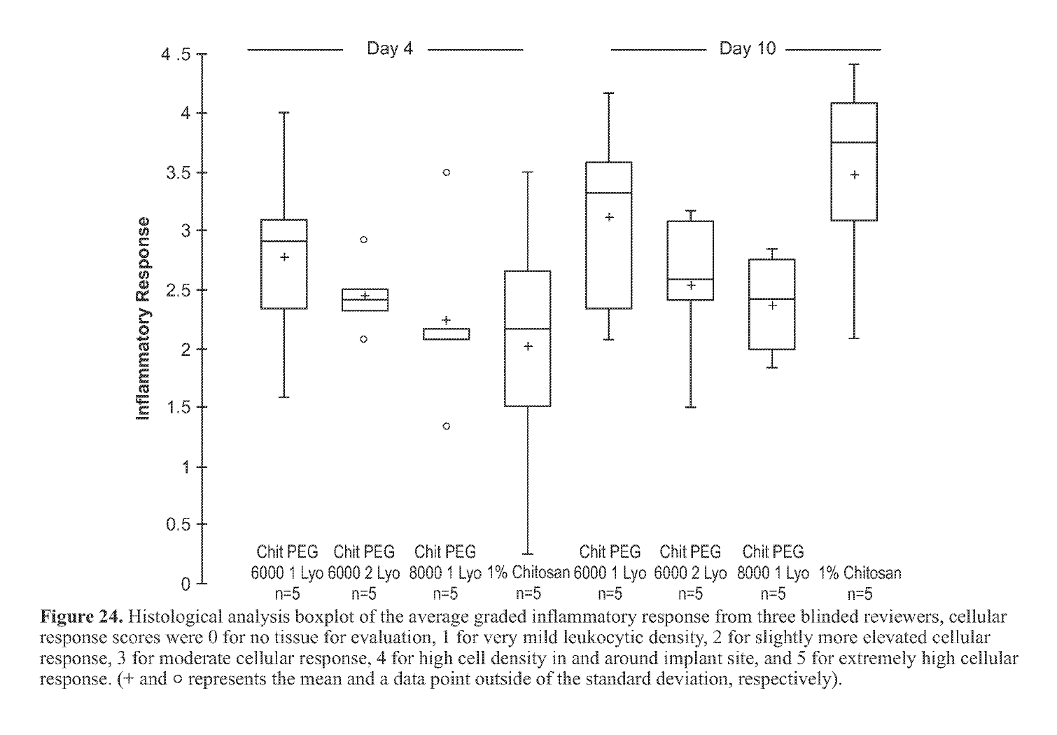

The percentage of implant area per defect area after 4 and 10 days of implantation is shown in FIG. 23, but none of the experimental groups, with regard to time, exhibited significant differences (p=0.449). The control 1% chitosan sponges and chitosan/PEG 6000 1 lyo sponges did exhibit significant differences in percent implant after 4 days of implantation (p=0.001). None of the sponges degraded completely after 10 days.

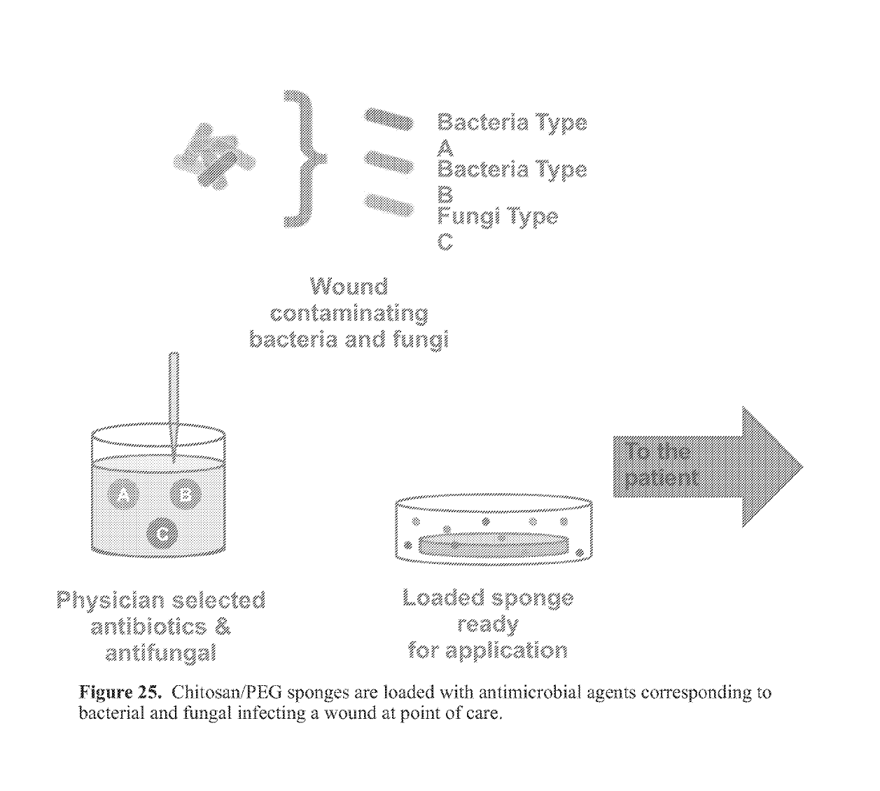

After 4 days of implantation, the chitosan/PEG 8000 1 lyo sponge exhibited the lowest average tissue response of the blended sponges (FIG. 24), although not statistically different (p=0.336). Similar to the implant percentage results, the chitosan/PEG 6000 1 lyo sponge displayed the highest average tissue response of all the sponges. After 10 days of implantation, the average tissue response increased by 20, 4, 5, and 72% for the 1% chitosan, chitosan/PEG 6000 1 lyo, chitosan/PEG 6000 2 lyo, and chitosan/PEG 8000 1 lyo sponges from 4 days of implantation, respectively.

While adding PEG to the chitosan sponges resulted in increased in vitro degradation, in vivo degradation results did not confirm this trend over the course of 10 days. However, similar to the in vitro degradation results, the chitosan/PEG 8000 1 lyo sponge demonstrated the lowest percent implant in the defect after ten days of implantation. Conversely, the chitosan/PEG 6000 2 lyo sponge demonstrated increased in vivo degradation than the chitosan/6000 1 lyo sponge, whereas the opposite was shown in vitro. These results demonstrate the difficulty of translation from in vitro studies to in vivo. Although chitosan has been reported to be biodegradable and biocompatible, wide data variations exist in the literature; ranging from minimal degradation after subcutaneous implantation to rapid degradation in a wound model (Stinner et al., J Orthop Trauma 2010; 24(9):592-7; Dash et al., Progress in Polymer Science 2011; 36(8):981-1014; Ma et al., Biomaterials 2003; 24(26):4833-41). There was also wide variation in results in this study which led to the blended sponges exhibiting statistical similarity in percent implant from the control chitosan sponges.

The increased in vivo inflammatory response from the chitosan/PEG 6000 1 lyo sponge after 4 and 10 days of implantation compared to the other blended sponges demonstrate that, in addition to reduced in vivo sponge degradation, the chitosan/PEG 6000 1 lyo sponge exhibited a more acute inflammatory response than the other two blended sponges, although not higher than control chitosan sponges. The moderate inflammatory responses observed in this study were lower than the tissue response to sutures and are similar to reported responses from chitosan coated titanium pins implanted in Sprague-Dawley rats (Norowski et al., Implant Dent 2011; 20(1):56-67).

### Example 11: In Vitro Elution of Amphotericin B and Vancomycin Dual Loaded Sponges

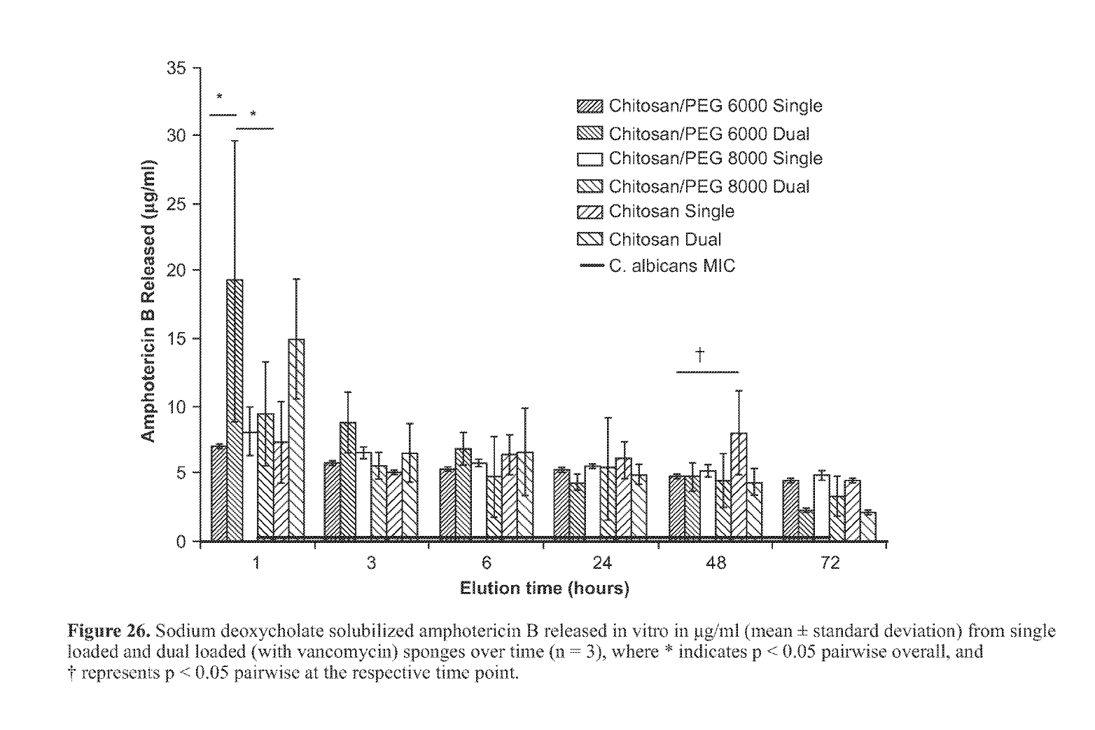

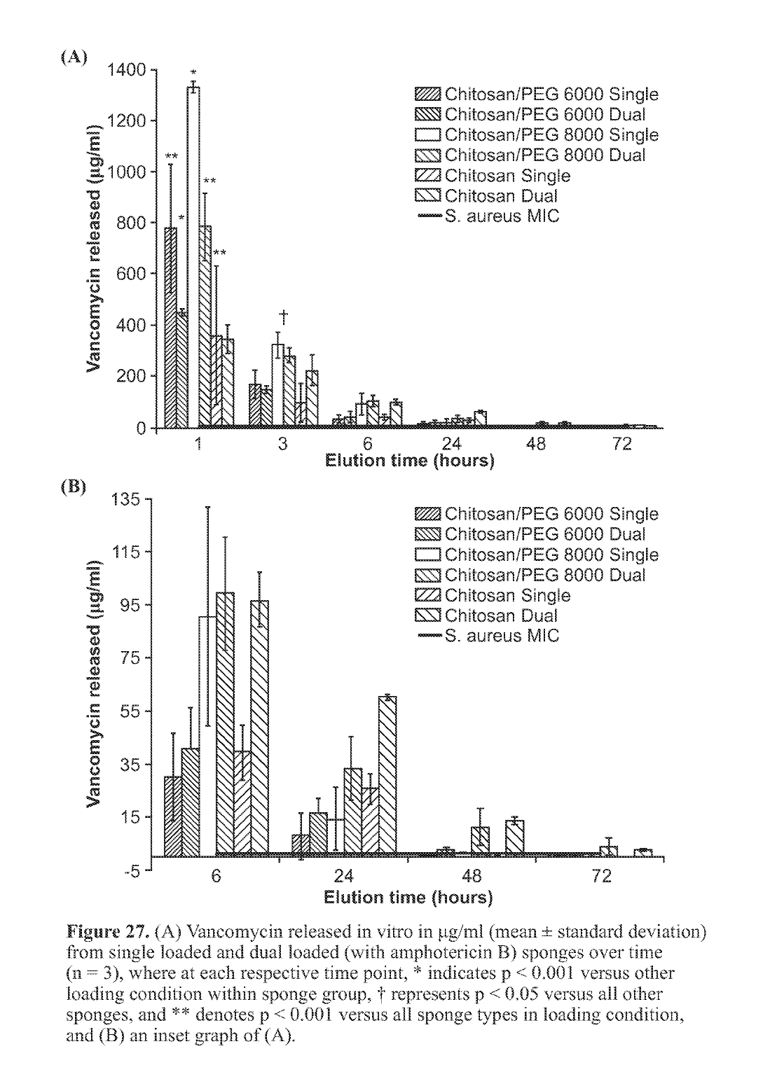

Chitosan-PEG sponges were made that could be loaded with antimicrobial agents, including hydrophobic antifungal agents (FIG. 25). After previous sponge formulation and material characterization, the selected chitosan/PEG sponges were evaluated for their in vitro amphotericin B and vancomycin release, both alone and in combination, as well as activity and cytocompatibility of the resulting antimicrobial eluates. The chitosan/PEG and chitosan sponges released amphotericin B in vitro, alone and in combination with the vancomycin, well above the amphotericin B minimum inhibitory concentration of Candida albicans over the 72 hour elution (FIG. 26). The dual-loaded sponges released more amphotericin B than the single-loaded sponges, especially after one hour of elution. Significant differences were detected in the antifungal release between the chitosan/PEG 6000 single- and dual-loaded sponges (p=0.003).

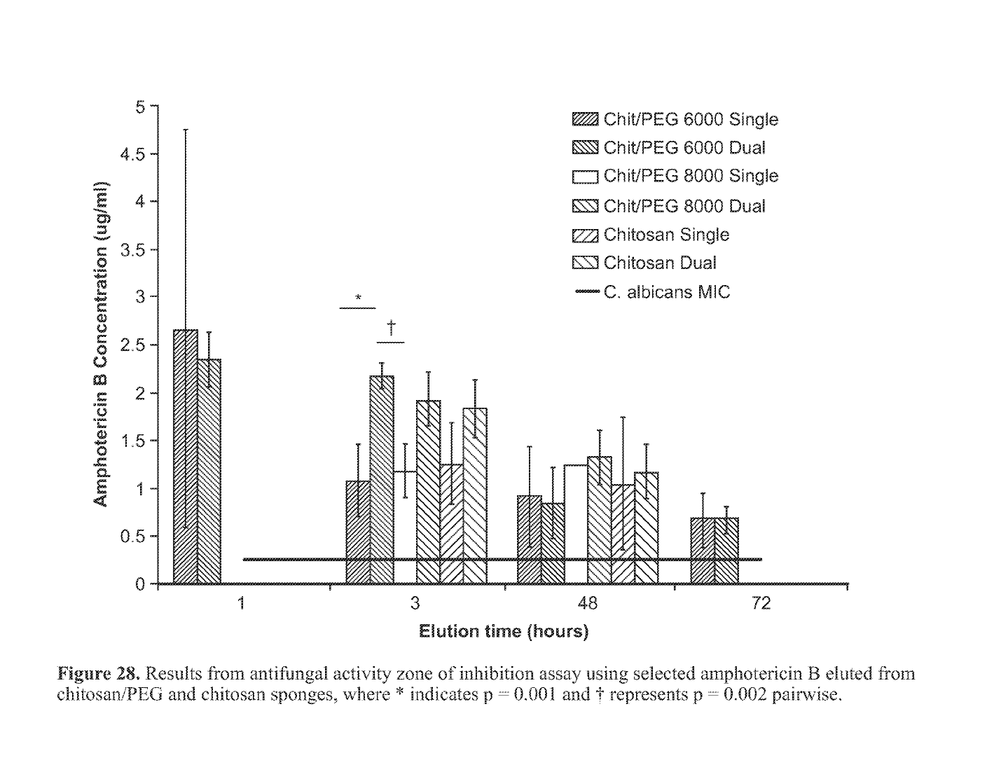

Unlike the antifungal elution, vancomycin release from single- and dual-loaded blended and chitosan sponges decreased quickly after the initial burst release (FIG. 27). All sponges released vancomycin at levels above the S. aureus MIC through 24 hours, but only the dual-loaded chitosan/PEG 8000 and chitosan sponges released vancomycin above the MIC through 72 hours of elution. The chitosan/PEG 8000 single loaded sponge also released significantly more vancomycin than the other dual-loaded sponges after one hour of elution than all other sponges after three hours. Vancomycin release exhibited contrasting behavior to the amphotericin B release in that the single-loaded sponges released more vancomycin than the dual-loaded.

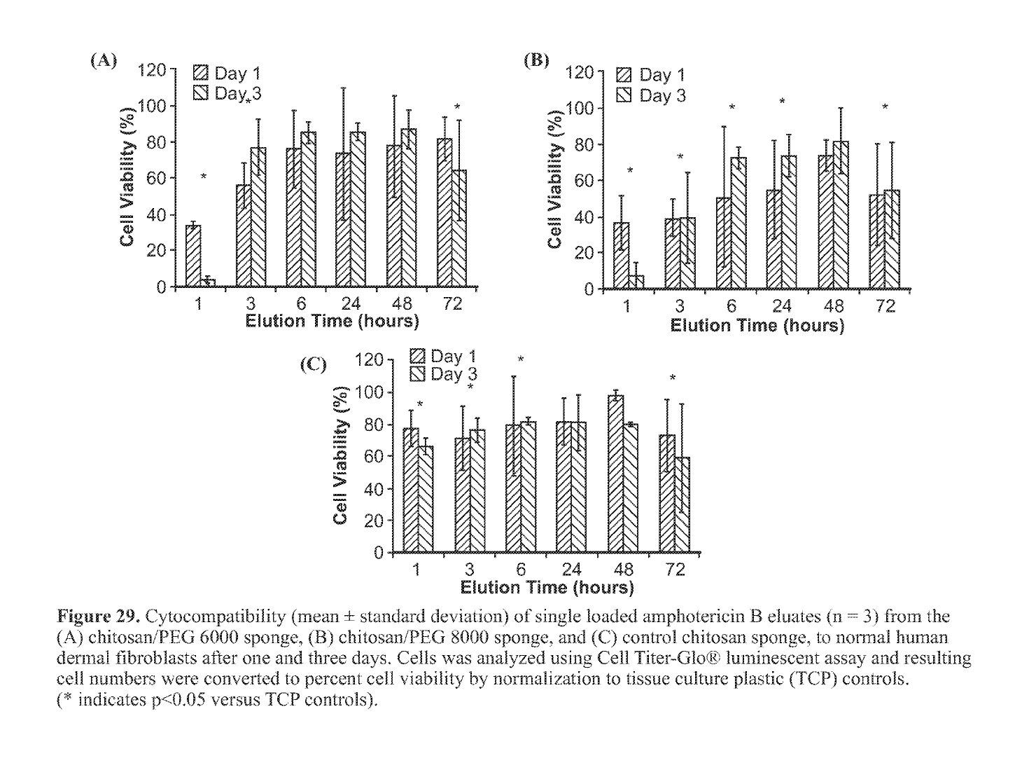

All selected amphotericin B eluates remained active against C. albicans at all-time points (FIG. 28). After three hours of elution, the same trend was found as in the antifungal elution data, where the dual-loaded sponges released more active amphotericin B than the single-loaded sponges. However, the chitosan/PEG 6000 dual-loaded sponge did not exhibit higher average levels of active antifungal than the same single-loaded sponge after one hour of elution (p=0.616). All sponges released vancomycin and were active against the bacteria over six hours of elution, and the dual-loaded chitosan sponges still inhibited bacterial growth through 24 hours (Table 2).

TABLE 2
Activity of 1:10 dilutions of vancomycin released (both single
loaded and dual loaded with amphotericin B) from blended
chitosan/PEG and control chitosan sponges against S. aureus

| | | Hours of Elution | | | | | |
|---|---|---|---|---|---|---|---|
| Loading | Sponge | 1 | 3 | 6 | 24 | 48 | 72 |
| Single | Chitosan/PEG | − | − | − | + | + | + |
| | 6000 2 lyo | | | | | | |
| Single | Chitosan/PEG | − | − | − | + | + | + |
| | 8000 1 lyo | | | | | | |
| Single | 1% Chitosan | − | − | − | + | + | + |
| Dual | Chitosan/PEG | − | − | − | + | + | + |
| | 6000 2 lyo | | | | | | |
| Dual | Chitosan/PEG | − | − | − | + | + | + |
| | 8000 1 lyo | | | | | | |
| Dual | 1% Chitosan | − | − | − | − | + | + |

− and + indicate no bacterial growth or the growth of bacteria, respectively (n = 3)

Confirming the HPLC and spectrophotometric quantification, the dual-loaded chitosan sponges released more active amphotericin B at 24 hours of elution than the other sponge groups.

Comparing the blended chitosan/PEG sponges to the limited previous literature on local antifungal delivery, there are some similarities and differences to published research on local antifungal delivery systems (Goss et al., J Arthroplasty 22:902-908; Kweon et al., Clin Orthop Relat Res 469:3002-3007; Kim et al., Eur J Pharm Sci 41:399-406; Cunningham et al., Clin Orthop Relat Res 470:2671-2676). While the blended sponges in this study still retained amphotericin B, both the chitosan/PEG 6000 and 8000 sponges released more antifungal in vitro in comparison to previous reports for amphotericin B loaded PMMA bone cement. Kweon et al. (Clin Orthop Relat Res 469:3002-3007) and Goss et al. (J Arthroplasty 22:902-908) have reported bone cement to release 0.45 and 0.05%, with and without a poragen, or 0.03% of loaded amphotericin B, respectively. However, the chitosan/PEG sponges released less antifungal than the 60% cumulative amphotericin B released after two days from a preloaded Pluronic® based copolymer gel (Kim et al., Eur J Pharm Sci 41:399-406). Additionally, the amphotericin B eluates from the sponges in this study are similar to the reported active amphotericin B eluted from PMMA bone cement (Cunningham et al., Clin Orthop Relat Res 470:2671-2676).

The in vitro local vancomycin release from the blended sponges can also be compared to previous studies on antibiotic loaded chitosan sponges (Noel et al., Clin Orthop Relat Res 468:2074-2080; Stinner et al., J Orthop Trauma 24:592-597; Parker et al., J Biomed Mater Res B Appl Biomater 101:110-123). Vancomycin release after one hour of elution, from single or dual loaded sponges, was 23-52% and 12-59% higher from the chitosan/PEG sponges than previously studied vancomycin loaded crosslinked chitosan sponges (Parker et al. J Biomed Mater Res B Appl Biomater 101:110-123). However, unlike the blended sponges, these previously studied crosslinked sponges released vancomycin at levels above the S. aureus MIC for up to 72 hours of elution (Parker et al. J Biomed Mater Res B Appl Biomater 101:110-123). The chitosan/PEG 8000 sponge, loaded with vancomycin only, initially released more vancomycin in vitro than previously reported for chitosan sponges made with lactic and acetic acid and loaded with 5 mg/ml vancomycin. However, the blended sponge released high enough levels of vancomycin to inhibit S. aureus growth up to 24 hours.

### Example 12: Cytocompatibility of Amphotericin B and Vancomycin Dual Loaded Sponges

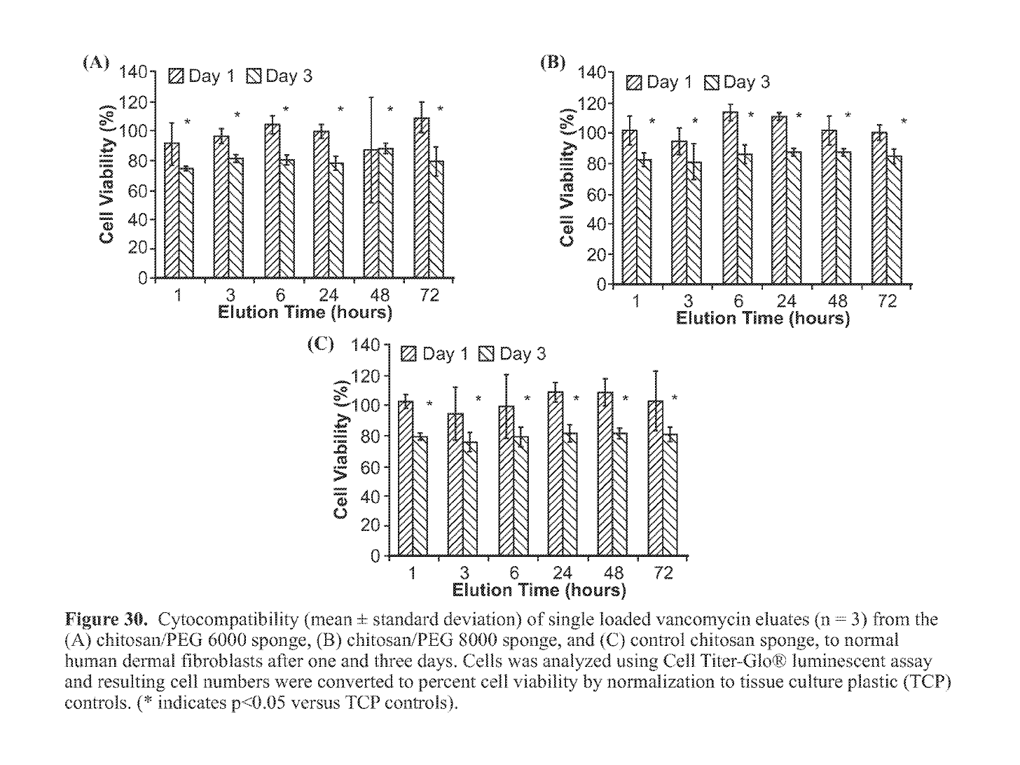

The one hour amphotericin B elution samples from single loaded chitosan/PEG sponges caused 35.17-38.33% and 58.81-62.57% decreases in cytocompatibility from the control tissue culture plastic plate after one and three days of treatment, respectively (FIGS. 29A-29C). While antifungal released from the blended sponges reduced cell viability after one hour of elution, previous research has confirmed amphotericin B's negative effects on cells, when exposed to high concentrations (Harmsen et al., Clin Orthop Relat Res 469:3016-3021). Harmsen and researchers have studied amphotericin B at typical locally delivered concentrations and found amphotericin B at 100 μg/ml caused fibroblast death after 5 hours, while 5 and 10 μg/ml caused abnormal cell morphology and reduced proliferation after 7 days and 5 hours and 7 days, respectively (Harmsen et al., Clin Orthop Relat Res 469:3016-3021). However, the amphotericin B eluates obtained from the same sponges after three hours of elution did not elicit as large of decreases in cell viability as the one hour eluates. An increase in cell viability occurred from one to three days of treatment for most time points, except for the one and 72 hour time points for all sponges and chitosan/PEG 6000 and chitosan sponges, respectively. As expected, vancomycin eluted from single-loaded chitosan/PEG and chitosan sponges supported higher cell viability than the released amphotericin B (FIGS. 30A-30C).

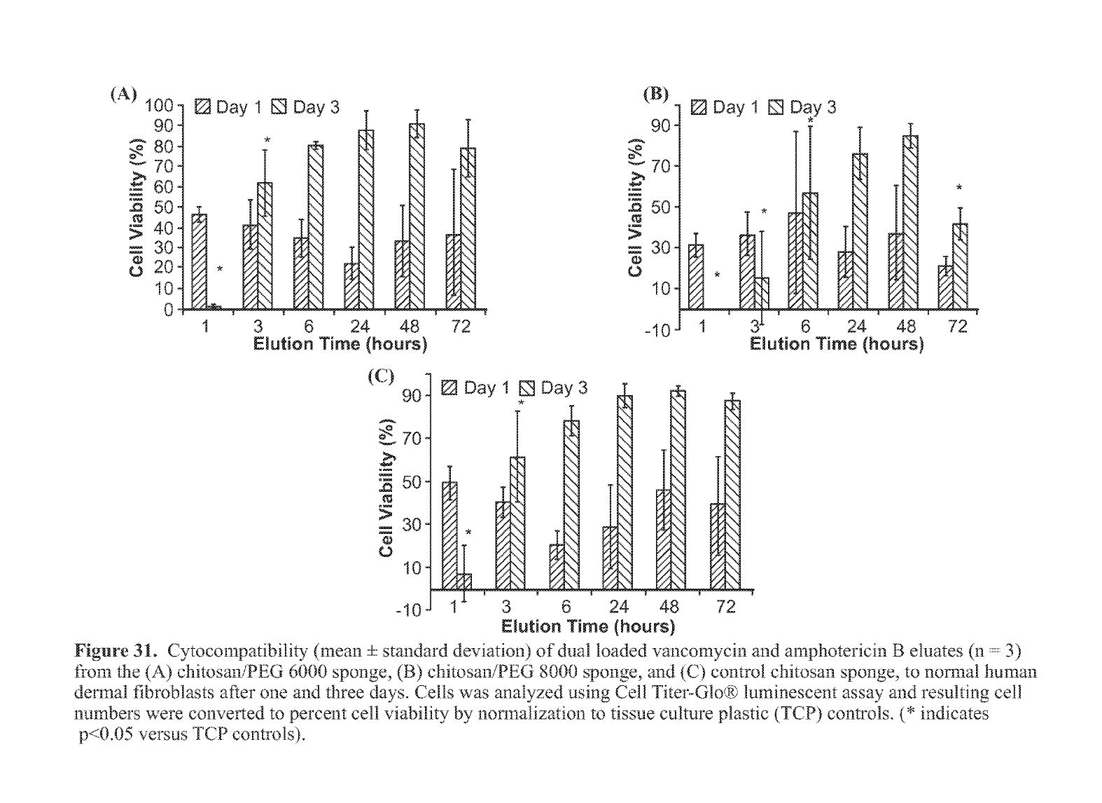

Combination vancomycin and amphotericin B eluted from all dual-loaded sponge types caused high decreases in cell viability after one day of treatment (FIGS. 31A-31C), but nostatistical difference was found over time for the elution time points (p=0.279). However, these same eluates exhibited higher cell viability after three days of treatment from six to 72 hours of elution from their respective one treatment day numbers. The one and three hour elution samples from the chitosan/PEG 6000, 8000, and chitosan sponges exhibited significantly lower cell viability than the controls, and the 72 hour elution sample from chitosan/PEG 8000 sponge also cause a significant decrease in cell viability (p<0.001).

### Example 13: Vancomycin Loaded Chitosan/PEG Sponges Inhibited Bacteria In Vivo

Staphylococcus aureus is a bacterial pathogen that is a primary cause of chronic wound infection. These infections are difficult to treat because they are associated with formation of a bacterial biofilm, which limits the effectiveness of many antibiotics. A biofilm consists of multiple layers of bacteria attached to a solid surface and encased in some form of extracellular matrix. The prevalence of biofilm-based infection, together with the continued emergence of bacterial strains that are resistant to commonly-used antibiotics, has led to the development of novel methods to prevent and treat these infections. A localized drug delivery system could potentially overcome the challenges that are associated with traditional drug treatment systems. Biodegradable drug delivery systems offer the ability to provide extended release of therapeutic agents while being resorbable, reducing the risk for biofilm attachment and the development of antibiotic resistance.

Chitosan is a naturally occurring biopolymer that has been used in several drug delivery systems both alone and in combination with other materials. The biomaterial chitosan is biodegradable, antibacterial, and allows for antibiotic storage and delivery. Polyethylene glycol is another biocompatible polymer that has been used to modify chitosan drug delivery systems, and exhibits low toxicity and enhanced cell growth.

Two new alternative chitosan drug delivery devices have been developed: a chitosan paste and a chitosan/polyethylene glycol sponge. Chitosan manufactured into a paste form offers the improvements of adhesion and full wound coverage for enhanced drug diffusion over prior chitosan sponge drug delivery devices. Adding polyethylene glycol to chitosan sponges has shown to increase sponge degradation in vitro, as compared to the degradation of the prior unmodified chitosan sponges.

Vancomycin and amikacin loaded chitosan paste and vancomycin loaded chitosan/polyethylene glycol sponges have been tested for their efficacy in preventing biofilm formation in mice with implanted catheters inoculated with S. aureus. These experiments were carried out in an established mouse model in which a small piece of catheter is implanted subcutaneously and then inoculated with the bacterial strains to be studied. The catheter is important because it provides the surface for bacterial attachment. This experimental set-up also mimics the clinical situation in which infections develop on indwelling medical devices including intravenous catheters.

Mice were anesthetized by inhalation of Isoflurane (0.5-3%). Anesthesia was confirmed by the lack of a toe pinch reflex. To initiate infection, mice were shaved on each flank before making a small (0.3 cm) incision in the skin. A 1 cm section of 14 gauge Teflon catheter was placed under the skin on each flank. At the time of catheter placement, chitosan paste loaded with vancomycin solution was injected by a taper tipped syringe adjacent to the catheter segment. In the other mice at the time of catheter placement, the chitosan/polyethylene glycol sponges loaded with vancomycin solution were placed with tweezers adjacent to the catheter segment. The incision was closed with surgical glue (VetBond) before injecting 1 cc of 10⁴ CFUs S. aureus into the lumen of the catheter. Any mice that had an adverse reaction to sedation (as evidenced by a failure to waken and return to full mobility within 30 min after surgery) were euthanized.

Implanted catheters and experimental drug delivery systems remained in place for 48 hours after surgery. At that point, mice were sacrificed as described below. The catheters were surgically removed from each flank and stored in a sterile saline solution. The catheters were processed in the lab and bacterial plate counts were performed for bacteriological confirmation of infection status as previously described (Beenken et al., 2004, Global gene expression in Staphylococcus aureus biofilms, Journal of Bacteriology, 186:4665-4684, Cassat et al., 2005).

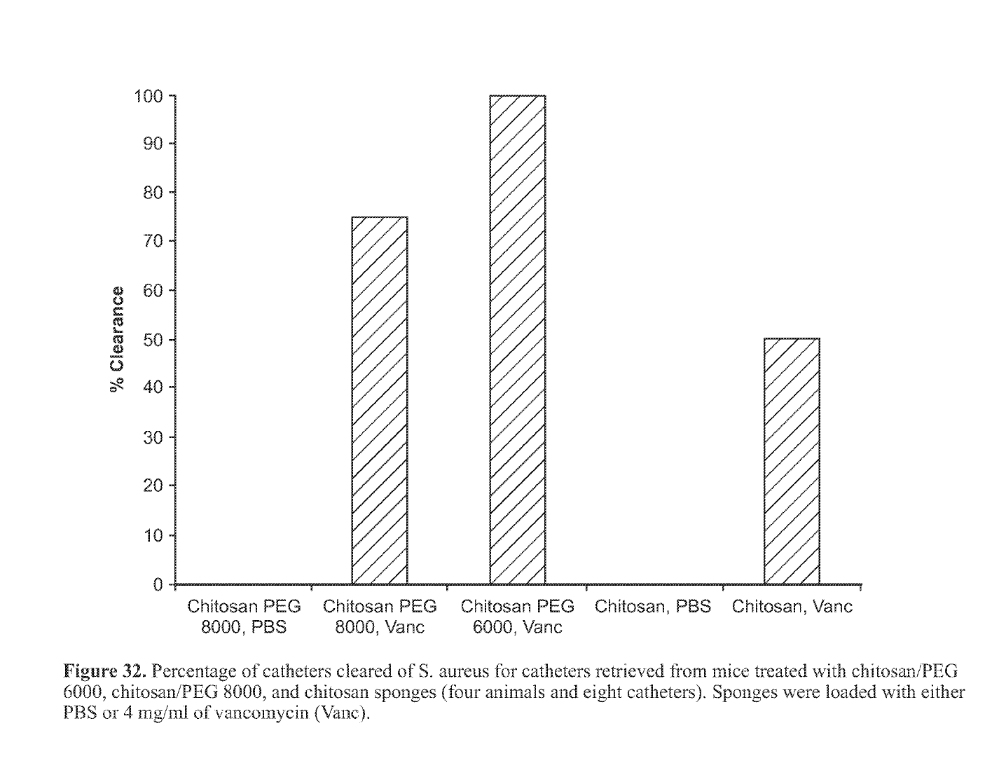

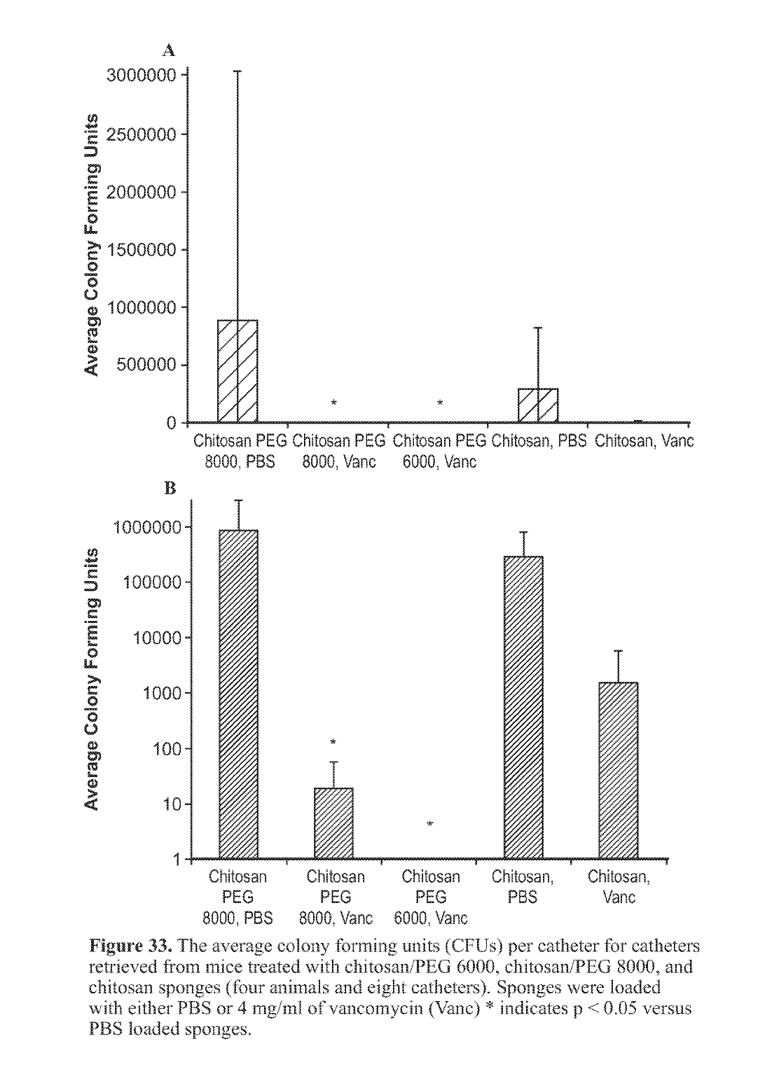

Significantly, clearance of Staphylococcus aureus biofilm on catheters was observed after forty-eight hours of treatment with vancomycin loaded chitosan/PEG sponges (FIG. 32). Treatment with the vancomycin loaded chitosan/PEG 6000 sponge resulted in 100% bacteria clearance from the catheters. Six of the eight catheters used with the vancomycin loaded chitosan/PEG 8000 sponges were cleared, while only 50% of the catheters treated with the vancomycin loaded chitosan (no PEG) sponges were cleared. A reduction in the average S. aureus colony forming units present on catheters in mice after 48 hours of treatment with PBS or Vanc loaded chitosan and chitosan/PEG sponges was observed (FIGS. 33A and 33B). The average colony forming units on the colonized catheters from the vancomycin loaded chitosan/PEG 8000 and chitosan sponge treatments were significantly lower than the CFUs from the PBS loaded chitosan/PEG 8000 sponge treatment (p=0.038 and p=0.032).

Thus, these results demonstrate that chitosan-PEG sponges can be used as wound treatment devices that can be tailored to treat bacterial and fungal infections by loading the appropriate antimicrobial agents at point of care (FIG. 13). Indeed, results show that in vivo blended chitosan sponges can prevent biofilm formation. Blended chitosan and polyethylene glycol sponges can be used to provide a vehicle for the local delivery of clinician-selected antifungals and/or antibiotics for use as an adjunctive therapy for the prevention of polymicrobial musculoskeletal wound infections.

The experiments described about were performed with the following methods.

### Chitosan-PEG Sponges

To make chitosan and polyethylene glycol blended sponges, PEG (6,000; 8,000; or 10,000 g/mol) (0.2-1% (w/v) was dissolved in a 1% (v/v) acidic solution (1% acetic acid or 1% 75:25 lactic:acetic acid). After PEG dissolution, chitosan (61% or 71% DDA; 0.5-1% (w/v)) was dissolved in the same solution. The solution was mixed on a stir plate (approximately one hour), and then the solution (25 mL) was poured into aluminum pans. The sponges were frozen at −20° C. or −80° C. (e.g., at least one hour at −80° C.) and then lyophilized in a freeze dryer (LabConco) for 48 hours. Optionally, chitosan-PEG sponges were neutralized in NaOH (e.g., 0.25M, 0.6M, or 1M NaOH), by soaking the sponges briefly in the base solution and then washing the sponges in large amounts of water until a neutral pH was reached. Optionally, the neutralized chitosan-PEG sponges were frozen again (e.g., at least one hour at −80 C for) and lyophilized a second time for 48 hours. In one embodiment, chitosan-PEG sponges made with 6,000 and 8,000 g/mol PEG, were neutralized or left unneutralized. The unneutralized chitosan-PEG sponges were only lyophilized once, yet still maintained a near neutral pH without neutralization. Chitosan and polyethylene glycol sponges were fabricated with weight ratios of chitosan to polyethylene glycol from 1:1, 2:1, and 4:1.

In a particular embodiment, blended chitosan and PEG sponges were made by dissolving 0.5% (w/v) of PEG (6,000 or 8,000 g/mol) in a 1% (v/v) acetic acid solution. After rapid PEG dissolution, 0.5% (w/v) of chitosan (250 kDa and 82.46±1.679 degree of deacetylation) was added to the same solution. Control chitosan sponges were made in the same manner, but with 1% (w/v) chitosan in 1% acetic acid solution. Chitosan/PEG and chitosan solutions were mixed and poured into 42 mL aluminum pans at a volume of 25 mL. Solutions were frozen at −80° C., followed by lyophilization in a LabConco (Kansas City, Mo.) FreeZone 2.5 Liter Benchtop Freeze Dry System. After lyophilization, the control chitosan sponges and the chitosan/PEG sponges with 6,000 g/mol PEG were neutralized in either 0.6 or 0.25 M NaOH, respectively. Sponges were soaked in the base solutions for approximately 1-2 minutes and then washed in copious amounts of water until a neutral pH was reached. Once neutralized, the chitosan and chitosan/PEG 6000 sponges were frozen and lyophilized again. The chitosan/PEG sponges with 8,000 g/mol PEG were only lyophilized once and not neutralized. For the two lyophilization treatment group, chitosan/PEG composite sponges were neutralized in 0.25 M NaOH and were labeled as chitosan/PEG 6000 2 lyo or chitosan/PEG 8000 2 lyo sponges. For the one the one lyophilization treatment group, chitosan/PEG composite sponges were not neutralized and were labeled as either chitosan/PEG 6000 1 lyo or chitosan/PEG 8000 1 lyo sponges. The concentrations of NaOH solutions were determined through previous formulation testing. After neutralization, the control chitosan and chitosan/PEG 6000 2 lyo sponges were frozen again at −80° C. and lyophilized one last time. All sponges were sterilized via low dose gamma irradiation at a 25-40 kGy dosage.

Chitosan and PEG sponges have been fabricated with chitosan from two different suppliers, Primex (Siglufjordur, Iceland) and Chitinor AS (Tromso, Norway), with a degree of deacetylation ranging from 61-82%. Polyethylene glycol (PEG) was obtained from Sigma Aldrich (St. Louis, Mo.). Sodium deoxycholate solubilized amphotericin B and vancomycin were obtained from Amresco (Solon, Ohio) and Fisher Scientific (Pittsburgh, Pa.), respectively. All of fabricated sponges were able to load and release amphotericin B.

Antimicrobial Loading

The chitosan-PEG sponges have been loaded with Fungizone®, a sodium deoxycholate form of amphotericin B soluble in water, amphotericin B, which is water insoluble, vancomycin (an antibiotic) and both vancomycin and Fungizone®. To load the antimicrobials in the sponges, a solution of the desired antimicrobial is made in sterile water and approximately 10 mL of the solution in poured on the sponges. The sponges are left in the solution and flipped over once, to ensure thorough absorbance of the antimicrobial solution. Sponges can then be placed in a vial of phosphate buffered saline for in vitro elution testing, or in an animal for in vivo testing.

### Antimicrobial Elution

In order to measure the antimicrobial elution in vitro over time, the chitosan-PEG sponges loaded with antimicrobials are placed in vials with 20 mL of PBS and incubated at 37° C. on a shaker. At 1, 3, 6, 24, 48, and 72 hours post antimicrobial loading, 1 mL aliquots of the PBS solution are removed and stored. The remaining PBS solution is then removed and refreshed with new PBS solution. The antimicrobial concentration at each time point can be determined by fluorescence polarization immunoassay, high pressure liquid chromatography, or ultraviolet visible spectroscopy.

In one embodiment, chitosan and chitosan/PEG sponges (128-264 mg; n=3) were loaded by swelling in a solution of freshly mixed Amphotericin B in ultrapure water (10 ml at 1 mg/ml). After approximately one minute, the amphotericin B solution not absorbed by the sponge was removed and measured. Sterile 1× phosphate buffered saline (20 ml PBS) was added to the sponges in Nalgene containers and then placed on a shaker in an incubator at 37° C. After 1, 3, 6, 24, 48, and 72 hours of elution, four 1 mL samples were removed from the sponge solution and the PBS was completely refreshed. Eluates were added 1:1 to dimethylsulfoxide (DMSO). Highly concentrated amphotericin B solutions, including the original 1 mg/mL solution, were diluted in DMSO multiple times. Concentration of Amphotericin B was determined via absorbance at 389 nm in a Biotek (Winooski, Vt.) Synergy H1 plate reader and concentrations determined using a standard curve (0.57 to 589 μg/ml).

In another embodiment, the in vitro local release of antifungals and/or antibiotics from the sponges was evaluated by loading the sponges with either sodium deoxycholate solubilized amphotericin B (1 mg/ml), vancomycin (4 mg/ml), or a combined solution of vancomycin (4 mg/ml) and sodium deoxycholate solubilized amphotericin B (1 mg/ml). All antimicrobial solutions were created in sterile Ultrapure water, and the dual vancomycin and amphotericin B solution was made by creating separate antibiotic and antifungal solutions first and then combining the two solutions into one. All antimicrobial solutions were shaken immediately prior to use and each solution (10 ml) was added to the sponges (n=3). After approximately one minute of absorbance, the antimicrobial solutions not absorbed by the sponges were removed and measured. Sponges were completely covered by sterile 1×PBS (20 mL) in 125 ml containers and incubated at 37° C. on a shaker. Elution samples were removed after 1, 3, 6, 24, 48, and 72 hours in 1 ml aliquots and the PBS was completely refreshed.

The concentration of single loaded amphotericin B released from the sponges was measured via high pressure liquid chromatography (HPLC) with a ThermoScientific BDS Hypersil C18 column. Amphotericin B was measured at 407 nm in a mobile phase of acetonitrile, methanol, and 10 mM sodium phosphate monobasic (41:10:49) (Egger et al., J Chromatogr B Biomed Sci Appl 760:307-313). Both single and dual-loaded vancomycin eluates were also measured with HPLC and the same column. The concentration of single loaded vancomycin released from the sponges was measured at 235 nm in a mobile phase of 35% acetonitrile and 65% buffer (0.08 M disodium phosphate and 0.013 M monosodium phosphate adjusted to pH 3 with phosphoric acid) (Smith et al., Clin Orthop Relat Res 471:3158-3164).

The concentrations of amphotericin B released from sponges dual-loaded with both vancomycin and amphotericin B were also measured with ultraviolet visible (UV-Vis) spectroscopy because the antifungal was not completely and accurately detected using the previously mentioned HPLC method. The dual eluates were added 1:1 to dimethylsulfoxide (DMSO) and well mixed. The highly concentrated stock solution was diluted in DMSO multiple times. Amphotericin B eluates from dual-loaded sponges were read via absorbance at 389 nm in a Biotek (Winooski, Vt.) Synergy H1 plate reader. Antifungal concentrations were calculated using an amphotericin B standard curve.

### In Vitro Sponge Degradation

Chitosan-PEG sponges are stored in a dessicator until they reach a consistent weight. Sponges are initially weighed and then placed in vials (e.g. 125 mL) with 35 mL of 1 mg/ml 2× crystallized chicken egg white lysozyme (e.g., from MP Biomedicals, Santa Ana, Calif.) in solution, for example PBS or water with penicillin (100 units/mL), streptomycin (100 mg/mL), amphotericin B (0.25 μg/mL) or 0.25 wt % vancomycin and daptomycin, respectively. Degradation samples (n=3) are placed on a rocking shaker in an incubator at 37° C. and the degradation solution is exchanged every 1-2 days. At each time interval (e.g., after 2, 4, 8, and 10 days), sponge samples from each sponge variation are removed from the degradation solution and soaked briefly in deionized water to stop any enzymatic degradation. Sponge samples are then placed in a vacuum oven at 60° C. for two days until dried thoroughly and are reweighed to obtain a final sponge weight. The percent remaining of the sponge is calculated using the following equation:

$$Percent remaining ( \% ) = ( \frac{Sponge weight at x days}{Initial dry sponge weight} ) \times 100$$

This study was a destructive study, so every time point represents a different sponge sample. The gentle, vacuum drying process eliminates the option of utilizing individual samples a second time, and therefore separate samples are analyzed for each replicate and time point.

### Fourier Transform Infrared Spectroscopy (FTIR)

Control and chitosan/PEG composite sponges were evaluated for chemical bond structure using attenuated total reflectance Fourier transform infrared spectroscopy (ATR-FTIR). A ThermoScientific (Waltham, Mass.) Nicolet iS10 FTIR spectrometer with a diamond ATR crystal was used to acquire the absorbance spectra (n=3), using 64 scans with a resolution of 4 cm⁻¹. The spectra were averaged and analyzed with the Thermo Scientific OMNIC™ Software Suite.

### X-Ray Diffraction (XRD)

X-ray diffraction (XRD) was used to assess crystallinity of the chitosan and chitosan/PEG sponges. A Bruker AXS (Maddison, Wis.) Advanced D8 X-ray diffractometer was utilized to obtain X-ray diffraction patterns with a Kα Cu radiation source at 40 kV and 40 mA. XRD data were obtained over a 20 range from 5° to 40° with a time/step of 0.2 seconds and a step size of 0.05°.

### Thermal Analysis

Differential scanning calorimetry (DSC) was used to evaluate endothermic and exothermic peaks of the chitosan powder, PEG 6000, PEG 8000, 1% chitosan sponges, and chitosan/PEG sponges. A Netzsch (Selb, Germany) 200 PC differential scanning calorimeter was used to scan the samples (n=3) from 40 to 400° C. at 20° C./min.

### Morphology

Chitosan and chitosan/PEG sponges were examined with a FEI/Philips XL30 Environmental Scanning Electron Microscope (SEM) at 15 kV using samples sputter-coated with 30 nm of Au/Pd. SEM images (n=3) were qualitatively evaluated for differences between sponges such as cross-section, surface texture and porosity.

### Molecular Weight Measurement (Viscosity)

Chitosan and chitosan/PEG sponges from the in vitro degradation 10 day time point (n=3) were dissolved in a sodium acetate and acetic acid solution. A Brookfield Programmable DV-II+Viscometer with a CPE-40 cone spindle and circulating water bath at 25° C. was used to measure the viscosity of sponge solutions at 50 rpm.

### In Vitro Antifungal Activity

Amphotericin B eluate samples were lyophilized (e.g., in foil) for one day, reconstituted in phosphate buffered saline (50 μL PBS), and used to inoculate blank discs. Standard concentrations of amphotericin B from 0 to 2.5 ng in 0.25 ng increments were also used to inoculate blank discs. A 0.5 McFarland standard of Candida albicans was prepared in sterile PBS and used to streak a Mueller Hinton agar plate in three different directions. An inoculated disc was added to each plate and plates were incubated overnight at 37° C. Zone of inhibition for each amphotericin B eluate sample was measured and compared to the standard curve to determine sample concentration.

### In Vitro Antibiotic Activity

Dilutions (1:10) of vancomycin eluates from single- and dual loaded sponges were inoculated with S. aureus (UAMS-1), using turbidity as a measure of growth inhibition. Vancomycin eluate samples were added in 200 μl increments to 1.75 ml of sterile trypticase soy broth (TSB) and 50 μl of S. aureus inoculum with approximately 2×10⁶ colony forming units (CFU) (Institute CaLS. 2009. Methods for dilution antimicrobial susceptibility for bacteria that grow aerobically-Eight Edition. Wayne, Pa.: Clinical and Laboratory Standards Institute). Sterile 1×PBS was also used as a positive growth control, while uninoculated. The inoculated elution samples were then mixed, incubated at 37° C. overnight, mixed again and read via absorbance at 530 nm in a spectrophotometer.

### In Vitro Cytocompatibility

The in vitro cytocompatibility of the chitosan sponge variations was tested on normal human dermal fibroblasts (NHDFs) obtained from Lonza (Walkersville, Md.) using a protocol modified from ASTM F813-07 “Standard Practice for Direct Contact Cell Culture Evaluation of Materials for Medical Devices” (2007). Normal human dermal fibroblasts (NHDFs; Lonza; Walkersville, Md.), (passages 4 through 7) were seeded at 1×10⁵ cells/mL and allowed to proliferate to near confluence on 12-well tissue culture plates in Dulbecco's Modified Eagle's Medium (DMEM) supplemented with 10% fetal bovine serum (FBS) and penicillin (100 units/mL), streptomycin (100 mg/mL), and amphotericin B (0.25 ng/mL), under standard cell culture conditions (37° C. and 5% CO₂ atmosphere). Cell culture medium was aspirated and refreshed with 1 mL of fresh medium, while chitosan, chitosan/PEG, and control polyurethane sponges were soaked in pre-warmed, sterile 1×PBS for approximately 20 minutes. A single 8 mm diameter test specimen (n=5) was gently placed in each well in direct contact with the cell monolayer. Cultures were incubated for either one or three days before biocompatibility was assessed with the Cell Titer-Glo® Luminescent Cell Viability assay (Promega; Madison, Wis.).

The in vitro cytocompatibility of the amphotericin B, vancomycin, and combination amphotericin B and vancomycin elution samples was tested on normal human dermal fibroblasts (NHDFs) obtained from Lonza (Walkersville, Md.). Cells (passages 9 through 12) were seeded at 1×10⁴ cells/well in Dulbecco's Modified Eagle's Medium (DMEM) supplemented with 10% fetal bovine serum (FBS), penicillin (100 units/mL), streptomycin (100 mg/mL), and amphotericin B (0.25 pg/ml) on 48 well polystyrene tissue culture plates (TCP) under standard cell culture conditions (37° C. and 5% CO₂). Elution samples were pre-warmed and diluted 1:1 in fresh medium (n=3), absent additional antibiotics and antimycotics. After cells reached near confluence, cell culture medium was aspirated from the plates and refreshed with eluate dilution samples. Cultures were incubated for either one or three days before biocompatibility was assessed with the Cell Titer-Glo® Luminescent Cell Viability assay (Promega; Madison, Wis.).

### In Vivo Sponge Degradation

In vivo degradation and biocompatibility of the chitosan and chitosan/PEG sponges was assessed in three to four month old male Sprague-Dawley rats (n=10). Rats were anesthetized with 2% isoflurane and were maintained under 4-5% isoflurane during surgery Two 1.5 cm incisions were made through the skin on each side of the midline (4 incisions total) and a 1.25 cm pouch was created in the latissimus dorsi muscle in each incision. One disk shaped implant, 9.5 mm in diameter and 5 mm in thickness, was implanted bilaterally in each muscle pouch. Implant sites were randomized and each rat received one of each of the following implant types: chitosan sponge, chitosan/PEG 6000 1 lyophilization (lyo), chitosan/PEG 6000 2 lyo, and chitosan/PEG 8000 1 lyo sponges. The muscle incision was sutured and the skin was closed with surgical staples. Five rats were euthanized by CO₂ inhalation at the four and ten day time points (n=5).

After euthanization, the implanted region and surrounding tissue were excised, placed in 10% formalin buffered phosphate for two days, and then bisected across the implant for quantitative and qualitative histological evaluation (paraffin-embedded and stained with hematoxylin and eosin). Surgical defect areas of the stained tissue sections were measured as the remaining sponge plus surrounding fibrous tissue using a Nikon inverted microscope Eclipse TE300 and BIOQUANT® OSTEO II image analysis software. The area of sponge was measured separately, divided by defect area, and converted to a percentage. Qualitative histological evaluation was conducted by three blinded reviewers based on cellular response. Histology was scored on a scale of 0 to 5 (low to high) in increments of 0.25, using a modified version of the Knodell Histological Activity Index (HAI).

### In Vivo Antibiotic Activity

In vivo prevention of S. aureus biofilm growth by vancomycin loaded chitosan/PEG sponges was assessed in NIH Swiss mice (n=20) (Beenkeen et al., J Bacteriol 186:4665-4684; Weiss et al., Antimicrob Agents Chemother 53:4096-4102). Mice were anesthestized with isoflurane in an environmental chamber and were shaved. A 0.3 cm incision was made in the skin on each flank and a 1 cm section of polytetrafluoroethylene (PTFE) catheter was placed under the skin on each flank. At the same time, the chitosan/PEG 6000, chitosan/PEG 8000, or control chitosan sponges loaded with either 4 mg/ml vancomycin or 1×PBS solutions (4 animals and 8 catheters per group) were placed adjacent to the catheters. Incisions were closed with surgical glue and 1 ml of 10⁴ CFUs of S. aureus (UAMS-1 strain) was injected into the lumen of the catheter. At 48 hours post-surgery, mice were sacrificed and catheters were surgically removed from each flank and placed in a sterile saline solution. The catheters were sonicated in PBS to remove adherent bacteria; the resulting solutions were serially diluted, plated on tryptic soy agar, and incubated at 37° C. overnight in order to count the number of CFUs of S. aureus recovered from each catheter.

### Statistical Analysis

Viscometry data and exothermic peak temperatures and areas from DSC were analyzed using one-way ANOVA with Holm Sidak post hoc analysis. Endothermic peak temperatures and areas from DSC data were analyzed with Kruskal-Wallis one-way ANOVA on ranks with Tukey's post hoc analysis. Elution, ZOI, cytocompatibility and tissue response were analyzed using two-way Analysis of Variance (ANOVA) with Holm Sidak post hoc analysis. Percent implant in the defect in the rat study was analyzed using Kruskal-Wallis one-way ANOVA with Dunn post hoc analysis. Statistical significance level was set at a=0.05.

For studies of dual-loaded chitosan/PEG sponges (vancomycin and amphotericin B) except for the percent clearance of bacteria from the mouse model, all data are presented as mean±standard deviation. One way ANOVA was used to analyze the swelling ratios of the sponges. In vitro antimicrobial elution, antifungal activity, and eluate cytocompatibility were analyzed using three way Analysis of Variance (ANOVA) with Holm Sidak post hoc analysis, with antimicrobial, sponge type, and elution time as the three factors. Colony forming unit data from the in vivo mouse model was analyzed with the nonparametric Kruskal-Wallis one way ANOVA on ranks with Tukey post hoc analysis. Statistical significance level was set at a=0.05.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for producing a biodegradable composition comprising chitosan and polyethylene glycol, the method comprising:
(a) dissolving polyethylene glycol having a molecular weight of at least about 2-12,000 g/mol and chitosan; and
(b) forming the mixture of chitosan and polyethylene glycol into a desired shape under conditions that reduce the water content by about 10%-100%.

2. (canceled)

3. A method for producing a biodegradable composition comprising chitosan, polyethylene glycol, and one or more agents selected by a clinician at a point of care, the method comprising:
(a) dissolving polyethylene glycol having a molecular weight of at least about 2-12,000 g/mol and chitosan in one or more acids in a solvent to form a mixture, and
(b) forming the mixture of chitosan and polyethylene glycol into a desired shape under conditions that reduce the water content by about 10%-100%;
(c) selecting one or more agents; and
(d) incorporating an effective amount of the agent into the composition at a point of care.

4. (canceled)

5. The method of claim 3, wherein the agent is an antiviral, antibacterial, antifungal, or anticancer agent.

6. (canceled)

7. (canceled)

8. The method of claim 3, wherein the water content is reduced by lyophilization in step (b).

9. The method of claim 3, wherein the method further comprises neutralizing the lyophilized sponge of step (b) in NaOH, washing the sponge to neutralize it, and then freezing the sponge and lyophilizing a second time.

10-12. (canceled)

13. The method of claim 3, wherein the desired shape is obtained by freezing the mixture of chitosan and polyethylene glycol in a mold and lyophilizing to form a sponge or by pouring the mixture of chitosan and polyethylene glycol into a thin layer and heating the chitosan to form a dehydrated film.

14-21. (canceled)

22. The method of claim 3, wherein the chitosan-PEG composition biodegrades over at least about 2-28 days when implanted in a subject.

23. (canceled)

24. A composition comprising chitosan and polyethylene glycol produced by the method of claim 3.

25. A wound management device comprising chitosan and polyethylene glycol produced by the method of claim 3.

26-34. (canceled)

35. The chitosan-PEG composition of claim 24, wherein the composition releases at least about 0.2-50 μg of an antimicrobial agent per hour.

36-39. (canceled)

40. A wound management device comprising the chitosan-PEG composition of claim 24.

41-53. (canceled)

54. A method for treating or preventing an infection in a subject at a site of trauma, the method comprising contacting the site with a wound management device comprising or consisting essentially of the chitosan-PEG composition of claim 24, and an effective amount of at least one agent selected at a point of care.

55-64. (canceled)

65. A method for the local delivery of an agent to a site, the method comprising contacting the site with the chitosan-PEG composition of claim 24 comprising an agent, thereby delivering the agent to the site.

66-68. (canceled)

69. The method of claim 65, wherein the chitosan-PEG composition releases about 100-200 μg in about 1 hour, about 200-400 μg in about 3 hours, about 250-500 μg in about 6 hours, about 350-700 μg in about 24 hours, about 500-800 μg in about 48 hours, about 600-1000 μg in about 72 hours.

70. A medical device for implantation comprising the chitosan composition of claim 23, wherein the chitosan-PEG composition is a film that adheres to the device, and comprising an effective amount of an agent.

71. (canceled)

73. The medical device of claim 70, wherein the medical device is a catheter or heart valve.

74. A kit comprising a chitosan-PEG composition of claim 23, for use in treating a trauma site or delivering an agent.

75-78. (canceled)
